SEQUENCE LISTING (170 sequences)

SEQ ID NO: 1 - DNA, 20 bp, Homo sapiens
  source 1..20: mol_type = unassigned DNA
tacgcacgcgtctgcagaag

SEQ ID NO: 2 - DNA, 20 bp, Homo sapiens
  source 1..20: mol_type = unassigned DNA
cgtctatgatgctggacgcg

SEQ ID NO: 3 - DNA, 20 bp, Homo sapiens
  source 1..20: mol_type = unassigned DNA
gacgtacttacttccctgat

SEQ ID NO: 4 - DNA, 16 bp, Homo sapiens
  source 1..16: mol_type = unassigned DNA
agaaggcgacgatgac

SEQ ID NO: 5 - DNA, 20 bp, Homo sapiens
  source 1..20: mol_type = unassigned DNA
ccgccaacagaattcatacg

SEQ ID NO: 6 - DNA, 20 bp, Homo sapiens
  source 1..20: mol_type = unassigned DNA
gcgtgcgtagaacttcatgc

SEQ ID NO: 7 - DNA, 2749 bp, Homo sapiens
  source 1..2749: mol_type = unassigned DNA
  CDS 76..1425
gcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcacactgtttcaaaagtta

SEQ ID NO: 8 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 9 - DNA, 1832 bp, Mus musculus
  source 1..1832: mol_type = unassigned DNA
  CDS 234..1580
gctgggtgctgcgcctcttacccccacctctaggcttccagaaagctcctggtgcattctccggcattctctgggcgtgagtcacgcaggtttgcagccagccccaaagggggtgtacttgagcagagcgctataaatagggcgcttccccggtgctcaccacccgcgtcaccaggaggagcgcactggagccaagccgcagaccggactccagattccaaggaggccacgccatgaagattctcctgctgtgcgtggcactgctgctgatctgggacaatggcatggtcctgggagagcaggaggtctctgacaatgagctccaagaactgtccactcaagggagtaggtatattaataaggagattcagaacgccgtccagggagtgaagcacataaaaactctcatagaaaaaaccaacgcagagcgcaagtccttgctcaacagtttagaggaagccaagaagaagaaagaggatgctctggaggacactagggattctgaaatgaagctgaaggcattcccggaagtgtgtaacgagaccatgatggccctctgggaggagtgcaagccctgcctgaagcatacctgcatgaagttctatgcacgtgtctgcaggagcggctcagggctggtcggccagcagctagaggagtttctaaaccagagctcacccttctacttctggatgaacggcgaccgcatcgactccctgctggagagtgaccggcagcagagccaagtcctggatgccatgcaggacagctttgctcgggcatctggcatcatagacacgctctttcaggaccggttcttcgcccgtgagcttcatgacccccactacttctcccccatcggcttcccacacaagaggcctcatttcttatatcccaagtcccgcttggtccgcagcctcatgtctccctcccactatgggcctccgagcttccacaacatgttccagcctttctttgagatgatccaccaggctcaacaggccatggatgtccagctccacagcccagccttccagttcccagacgtggatttcttaagagaaggtgaagatgaccgcactgtgtgcaaggagatccgccgcaactccacaggatgcctgaagatgaagggccagtgtgaaaagtgccaggagatcttgtctgtggactgttcaaccaacaatcctgcccaggctaacctgcgccaggagctgaacgactcgctccaggtggccgagaggctgacagagcagtacaaggagctgctgcagtccttccagtcgaagatgctcaacacctcatccctgctggagcagctgaacgaccagttcaactgggtgtcccagctggctaacctcacacagggcgaagacaagtactaccttcgggtctccaccgtgaccacccattcctctgactcagaggtcccctcccgtgtcactgaggtggtggtgaagctgtttgactctgaccccatcacagtggtgttaccagaagaagtctctaaggataaccctaagtttatggacacagtggcggagaaggcgctacaggaataccgcaggaaaagccgtgcggaatgagatagaagcatcaccttcctatatgtaggagtgtctgggagggaatctcccagctccccgaggtggctgcagacccctagagaactccacatgtctccaggcgagtaggcctcacccaagcagcctgcccttcctctggattctgtacactaatgcctgcacttgctgctctcgggaagaactgattcccccacgcaactaatccaataaaagccgccttgcgatacgtgtaaaaaaaaaaaaaaaaaaaaa

SEQ ID NO: 10 - protein, 448 aa, Mus musculus
MKILLLCVALLLIWDNGMVLGEQEVSDNELQELSTQGSRYINKEIQNAVQGVKHIKTLIEKTNAERKSLLNSLEEAKKKKEDALEDTRDSEMKLKAFPEVCNETMMALWEECKPCLKHTCMKFYARVCRSGSGLVGQQLEEFLNQSSPFYFWMNGDRIDSLLESDRQQSQVLDAMQDSFARASGIIDTLFQDRFFARELHDPHYFSPIGFPHKRPHFLYPKSRLVRSLMSPSHYGPPSFHNMFQPFFEMIHQAQQAMDVQLHSPAFQFPDVDFLREGEDDRTVCKEIRRNSTGCLKMKGQCEKCQEILSVDCSTNNPAQANLRQELNDSLQVAERLTEQYKELLQSFQSKMLNTSSLLEQLNDQFNWVSQLANLTQGEDKYYLRVSTVTTHSSDSEVPSRVTEVVVKLFDSDPITVVLPEEVSKDNPKFMDTVAEKALQEYRRKSRAE

SEQ ID NO: 11 - DNA, 21 bp, Homo sapiens
  source 1..21: mol_type = other DNA
cagcagcagagtcttcatcat

SEQ ID NO: 12 - DNA, 83 bp, Homo sapiens
  source 1..83: mol_type = other DNA
ccttggagtaaagtagcagcacataatggtttgtggattttgaaaaggtgcaggccatattgtgctgcctcaaaaatacaagg

SEQ ID NO: 13 - DNA, 84 bp, Mus musculus
  source 1..84: mol_type = other DNA
cccttggagtaaagtagcagcacataatggtttgtggatgttgaaaaggtgcaggccatactgtgctgcctcaaaatacaagga

SEQ ID NO: 14 - DNA, 98 bp, Homo sapiens
  source 1..98: mol_type = other DNA
ttgaggccttaaagtactgtagcagcacatcatggtttacatgctacagtcaagatgcgaatcattatttgctgctctagaaatttaaggaaattcat

SEQ ID NO: 15 - DNA, 89 bp, Homo sapiens
  source 1..89: mol_type = other DNA
gtcagcagtgccttagcagcacgtaaatattggcgttaagattctaaaattatctccagtattaactgtgctgctgaagtaaggttgac

SEQ ID NO: 16 - DNA, 84 bp, Homo sapiens
  source 1..84: mol_type = other DNA
gtcagaataatgtcaaagtgcttacagtgcaggtagtgatatgtgcatctactgcagtgaaggcacttgtagcattatggtgac

SEQ ID NO: 17 - DNA, 71 bp, Homo sapiens
  source 1..71: mol_type = other DNA
tgttctaaggtgcatctagtgcagatagtgaagtagattagcatctactgccctaagtgctccttctggca

SEQ ID NO: 18 - DNA, 72 bp, Homo sapiens
  source 1..72: mol_type = other DNA
tgtcgggtagcttatcagactgatgttgactgttgaatctcatggcaacaccagtcgatgggctgtctgaca

SEQ ID NO: 19 - DNA, 84 bp, Homo sapiens
  source 1..84: mol_type = other DNA
ggccagtgttgagaggcggagacttgggcaattgctggacgctgccctgggcattgcacttgtctcggtctgacagtgccggcc

SEQ ID NO: 20 - DNA, 81 bp, Homo sapiens
  source 1..81: mol_type = other DNA
cttcaggaagctggtttcatatggtggtttagatttaaatagtgattgtctagcaccatttgaaatcagtgttcttggggg

SEQ ID NO: 21 - DNA, 81 bp, Homo sapiens
  source 1..81: mol_type = other DNA
cttctggaagctggtttcacatggtggcttagatttttccatctttgtatctagcaccatttgaaatcagtgttttaggag

SEQ ID NO: 22 - DNA, 85 bp, Homo sapiens
  source 1..85: mol_type = other DNA
cgctggcgacgggacattattacttttggtacgcgctgtgacacttcaaactcgtaccgtgagtaataatgcgccgtccacggca

SEQ ID NO: 23 - DNA, 88 bp, Homo sapiens
  source 1..88: mol_type = other DNA
caccttgtcctcacggtccagttttcccaggaatcccttagatgctaagatggggattcctggaaatactgttcttgaggtcatggtt

SEQ ID NO: 24 - DNA, 87 bp, Homo sapiens
  source 1..87: mol_type = other DNA
agcttccctggctctagcagcacagaaatattggcacagggaagcgagtctgccaatattggctgtgctgctccaggcagggtggtg

SEQ ID NO: 25 - DNA, 97 bp, Mus musculus
  source 1..97: mol_type = other DNA
tttcctgctggctgcgggtgctctgactaggttgcactactgtgctgtgagaagcagtgcaatggtattgtcaaagcatctgggaccagcctcgaag

SEQ ID NO: 26 - DNA, 95 bp, Homo sapiens
  source 1..95: mol_type = other DNA
ctcatctgtctgttgggctggaggcagggcctttgtgaaggcgggtggtgctcagatcgcctctgggcccttcctccagccccgaggcggattca

SEQ ID NO: 27 - DNA, 94 bp, Homo sapiens
  source 1..94: mol_type = other DNA
ctttggcgatcactgcctctctgggcctgtgtcttaggctctgcaagatcaaccgagcaaagcacacggcctgcagagaggcagcgctctgccc

SEQ ID NO: 28 - DNA, 98 bp, Homo sapiens
  source 1..98: mol_type = other DNA
cgaggggatacagcagcaattcatgttttgaagtgttctaaatggttcaaaacgtgaggcgctgctataccccctcgtggggaaggtagaaggtgggg

SEQ ID NO: 29 - DNA, 87 bp, Homo sapiens
  source 1..87: mol_type = other DNA
gaaagcgctttggaatgacacgatcactcccgttgagtgggcacccgagaagccatcgggaatgtcgtgtccgcccagtgctctttc

SEQ ID NO: 30 - DNA, 73 bp, Homo sapiens
  source 1..73: mol_type = other DNA
acttggagagaggctggccgtgatgaattcgattcatcaaagcgagtcatacacggctctcctctcttttagt

SEQ ID NO: 31 - DNA, 112 bp, Homo sapiens
  source 1..112: mol_type = other DNA
ccaccccggtcctgctcccgccccagcagcacactgtggtttgtacggcactgtggccacgtccaaaccacactgtggtgttagagcgagggtgggggaggcaccgccgagg

SEQ ID NO: 32 - DNA, 101 bp, Homo sapiens
  source 1..101: mol_type = other DNA
gcgagaagatctcatgctgtgactctctggagggaagcactttctgttgtctgaaagaaaacaaagcgcttctctttagagtgttacggtttgagaaaagc

SEQ ID NO: 33 - DNA, 85 bp, Homo sapiens
  source 1..85: mol_type = other DNA
ctcaggctgtgaccctccagagggaagtactttctgttgtctgagagaaaagaaagtgcttccctttggactgtttcggtttgag

SEQ ID NO: 34 - DNA, 85 bp, Homo sapiens
  source 1..85: mol_type = other DNA
ctcaagctgtgactctccagagggatgcactttctcttatgtgaaaaaaaagaaggcgcttccctttagagcgttacggtttggg

SEQ ID NO: 35 - DNA, 96 bp, Homo sapiens
  source 1..96: mol_type = other DNA
cctcagtaagaccaagctcagtgtgccatttccttgtctgtagccatgtctatgggctcttgagttggccatctgagtgagggcctgcttattcta

SEQ ID NO: 36 - DNA, 96 bp, Homo sapiens
  source 1..96: mol_type = other DNA
cagtgctggggtctcaggaggcagcgctctcaggacgtcaccaccatggcctgggctctgctcctcctcaccctcctcactcagggcacaggtgat

SEQ ID NO: 37 - DNA, 77 bp, Homo sapiens
  source 1..77: mol_type = other DNA
gtgtgcatttgcaggaacttgtgagtctcctattgaaaatgaacaggagactgatgagttcccgggaacacccacaa

SEQ ID NO: 38 - DNA, 77 bp, Homo sapiens
  source 1..77: mol_type = other DNA
ggaagtgccctacttggaaaggcatcagttgcttagattacatgtaactattccctttctgagtagagtaagtctta

SEQ ID NO: 39 - DNA, 81 bp, Homo sapiens
  source 1..81: mol_type = other DNA
atggcgttttccctctccctgtcctggactggggtcagactgtgccccgaggagaagcagcagagaataggactacgtcat

SEQ ID NO: 40 - DNA, 73 bp, Homo sapiens
  source 1..73: mol_type = other DNA
gtgggcgggggcaggtgtgtggtgggtggtggcctgcggtgagcagggccctcacacctgcctcgccccccag

SEQ ID NO: 41 - DNA, 102 bp, Homo sapiens
  source 1..102: mol_type = other DNA
gtgggagggcccaggcgcgggcaggggtgggggtggcagagcgctgtcccgggggcggggccgaagcgcggcgaccgtaactccttctgctccgtcccccag

SEQ ID NO: 42 - DNA, 97 bp, Homo sapiens
  source 1..97: mol_type = other DNA
ggtgggaggattgcttgagcctggaagctggagcctgcagtgaactatcattgtgccactgtactccagcctaggcaacaaaatgaaatcctgtcta

SEQ ID NO: 43 - DNA, 80 bp, Homo sapiens
  source 1..80: mol_type = other DNA
cctctgtgagaaagggtgtgggggagaggctgtcttgtgtctgtaagtatgccaaacttattttccccaaggcagaggga

SEQ ID NO: 44 - DNA, 78 bp, Homo sapiens
  source 1..78: mol_type = other DNA
tgtcctctggggactcagcttgctctggctgctggattgaattagctgcaggaccaagatgagcccttggtggagaca

SEQ ID NO: 45 - DNA, 81 bp, Homo sapiens
  source 1..81: mol_type = other DNA
ggacaggcacctgaggctctgttagccttggctctgggtcctgctccttagagcagaggcagagaggctcagggtctgtct

SEQ ID NO: 46 - DNA, 90 bp, Homo sapiens
  source 1..90: mol_type = other DNA
ggcgcctctgcagctccggctccccctggcctctcgggaactacaagtcccagggggcctggcggtgggcggcgggcggaagaggcgggg

SEQ ID NO: 47 - DNA, 74 bp, Homo sapiens
  source 1..74: mol_type = other DNA
ctttattgagtccctactatgttccaggcactgggtatcgtaggtgcctggaacatagtagggactcaataaag

SEQ ID NO: 48 - DNA, 68 bp, Homo sapiens
  source 1..68: mol_type = other DNA
tattgagtccctactatgttccaggcacctacgatacccagtgcctggaacatagtagggactcaata

SEQ ID NO: 49 - DNA, 74 bp, Homo sapiens
  source 1..74: mol_type = other DNA
gtgcagagttattgcctctgttctaacacaagactaggcttccctgtgttagaataggggcaataactctgcac

SEQ ID NO: 50 - DNA, 77 bp, Homo sapiens
  source 1..77: mol_type = other DNA
cctggggggcggggagagaacgcagtgacgtctggccgcgtgcgcatgtcgggcgctttctcctccccctacccagg

SEQ ID NO: 51 - DNA, 76 bp, Homo sapiens
  source 1..76: mol_type = other DNA
ggggtcacctctctggccgtctaccttccacactgacaagggccgtggggacgtagctggccagacaggtgacccc

SEQ ID NO: 52 - DNA, 87 bp, Homo sapiens
  source 1..87: mol_type = other DNA
gggactggggatgaggaggcatcttgagaaatggaaggaatgggatctacttccagttcactagaggcgtcctgacacccctagctc

SEQ ID NO: 53 - DNA, 99 bp, Homo sapiens
  source 1..99: mol_type = other DNA
tctacaagcagatacaaggatgcccttgtacacaacacacgtgctgcttgtatagacatgagtgttgtctacgagggcatccttgtgtctgtgtgtgtg

SEQ ID NO: 54 - DNA, 94 bp, Homo sapiens
  source 1..94: mol_type = other DNA
gaatccggtccgtacaaactctgctgtgttgaatgattggtgagtttgtttgctcattgattgaatcactgcagagtttgtacggaccggattc

SEQ ID NO: 55 - DNA, 76 bp, Homo sapiens
  source 1..76: mol_type = other DNA
cttgggaggagggtggggtggctcctctgcagtgagtaggtctgcctggagctactccaccatctcccccagcccc

SEQ ID NO: 56 - DNA, 101 bp, Homo sapiens
  source 1..101: mol_type = other DNA
gatgggccccttgtgtcctgaattgggtgggggctctgagtggggaaagtgggggcctaggggaggtcacagttgggtctaggggtcaggagggcccagga

SEQ ID NO: 57 - DNA, 53 bp, Homo sapiens
  source 1..53: mol_type = other DNA
ggtgggggttggaggcgtgggttttagaacctatccctttctagccctgagca

SEQ ID NO: 58 - DNA, 63 bp, Homo sapiens
  source 1..63: mol_type = other DNA
gcatgctgggcgaggctggcatctagcacaggcggtagatgcttgctcttgccattgcaatga

SEQ ID NO: 59 - DNA, 62 bp, Homo sapiens
  source 1..62: mol_type = other DNA
ggtagggggcgggctccggcgctgggaccccactagggtggcgccttggccccgccccgccc

SEQ ID NO: 60 - DNA, 73 bp, Homo sapiens
  source 1..73: mol_type = other DNA
ggaggctgggctgggacggacacccggcctccactttctgtggcaggtacctcctccatgtcggcccgccttg

SEQ ID NO: 61 - DNA, 75 bp, Homo sapiens
  source 1..75: mol_type = other DNA
tttactctgaactagctctgtggatcctgacagacagcctgatagacaggatccacagagctagtccagagtaaa

SEQ ID NO: 62 - DNA, 79 bp, Homo sapiens
  source 1..79: mol_type = other DNA
ctcgaggtgctgggggacgcgtgagcgcgagccgcttcctcacggctcggccgcggcgcgtagcccccgccacatcggg

SEQ ID NO: 63 - DNA, 78 bp, Homo sapiens
  source 1..78: mol_type = other DNA
gggcccagaagggggcgcagtcactgacgtgaagggaccacatcccgcttcatgtcagtgactcctgccccttggtct

SEQ ID NO: 64 - DNA, 82 bp, Homo sapiens
  source 1..82: mol_type = other DNA
gctacggggagcggggaggaagtgggcgctgcttctgcgttatctggaaggagcagcccactcctgtcctgggctctgtggt

SEQ ID NO: 65 - DNA, 60 bp, Homo sapiens
  source 1..60: mol_type = other DNA
ggcaggagggctgtgccaggttggctgggccaggcctgacctgccagcacctccctgcag

SEQ ID NO: 66 - DNA, 70 bp, Homo sapiens
  source 1..70: mol_type = other DNA
ccctgccagtgctgggggccacatgagtgtgcagtcatccacacacaagtggcccccaacactggcaggg

SEQ ID NO: 67 - DNA, 61 bp, Homo sapiens
  source 1..61: mol_type = other DNA
cctgcggggacaggccagggcatctaggctgtgcacagtgacgcccctcctgcccccacag

SEQ ID NO: 68 - DNA, 76 bp, Homo sapiens
  source 1..76: mol_type = other DNA
aggtgcacgctctagcggggattccaatattgggccaattcccccaatgttggaatcctcgctagagcgtgcactt

SEQ ID NO: 69 - DNA, 59 bp, Homo sapiens
  source 1..59: mol_type = other DNA
tatccagcttgttactatatgctttttaaatggggcacagagtgacaagctggttaaag

SEQ ID NO: 70 - DNA, 55 bp, Homo sapiens
  source 1..55: mol_type = other DNA
gggggctggggcgcggggaggtgctaggtcggcctcggctcccgcgccgcacccc

SEQ ID NO: 71 - DNA, 72 bp, Homo sapiens
  source 1..72: mol_type = other DNA
acaggtgggagagcagggtattgtggaagctccaggtgccaaccacctgcctctattccccactctccccag

SEQ ID NO: 72 - DNA, 63 bp, Homo sapiens
  source 1..63: mol_type = other DNA
tcttcttgggggtgaggttggtgtctggccccagcagcccagactgagcctctctctctccag

SEQ ID NO: 73 - DNA, 61 bp, Homo sapiens
  source 1..61: mol_type = other DNA
ctgactcccagggtctggtcagagttgctgagtgggttgatctctggtctttccttgacag

SEQ ID NO: 74 - DNA, 66 bp, Homo sapiens
  source 1..66: mol_type = other DNA
tcaagacggggagtcaggcagtggtggagatggagagccctgagcctccactctcctggcccccag

SEQ ID NO: 75 - DNA, 65 bp, Homo sapiens
  source 1..65: mol_type = other DNA
ctcctctgggggtggggggctgggcgtggtggacagcgatgcatccctcgccttctcaccctcag

SEQ ID NO: 76 - DNA, 66 bp, Homo sapiens
  source 1..66: mol_type = other DNA
aggattctgccataggaagcttggagtggaactgacctgccccctttctctctgactccatggcag

SEQ ID NO: 77 - DNA, 63 bp, Homo sapiens
  source 1..63: mol_type = other DNA
gaggtgtaggggaggttgggccagggatgccttcactgtgtctctctggtcttgccaccccag

SEQ ID NO: 78 - DNA, 56 bp, Homo sapiens
  source 1..56: mol_type = other DNA
cagggaagcagcagtggcaagactcctaggtcacggaagtcctgcttctgttgcag

SEQ ID NO: 79 - DNA, 60 bp, Homo sapiens
  source 1..60: mol_type = other DNA
caggctgggggctggatggggtagagtaggagagcccactgaccccttctgtctccctag

SEQ ID NO: 80 - DNA, 70 bp, Homo sapiens
  source 1..70: mol_type = other DNA
gtccctgggggctgggatgggccatggtgtgctctgatccccctgtggtctcttggcccccaggaactcc

SEQ ID NO: 81 - DNA, 63 bp, Homo sapiens
  source 1..63: mol_type = other DNA
cccagcagagggaatacagagggcaatcaggactgggtcattctctctgtctttctctctcag

SEQ ID NO: 82 - DNA, 78 bp, Homo sapiens
  source 1..78: mol_type = other DNA
cccgcagaggctgagaaggtgatgttggctcaagaaagggagatagatggtagcccatcacctttccgtctcccctag

SEQ ID NO: 83 - DNA, 59 bp, Homo sapiens
  source 1..59: mol_type = other DNA
ctgtgtcggggagtctggggtccggaattctccagagcctctgtgcccctacttcccag

SEQ ID NO: 84 - DNA, 65 bp, Homo sapiens
  source 1..65: mol_type = other DNA
gtctcctggggggaggagaccctgctctccctggcagcaagcctctcctgcccttccagattagc

SEQ ID NO: 85 - DNA, 86 bp, Homo sapiens
  source 1..86: mol_type = other DNA
ggggctgggggtgtggggagagagagtgcacagccagctcagggattaaagctctttctctctctctctctcccacttccctgcag

SEQ ID NO: 86 - DNA, 61 bp, Homo sapiens
  source 1..61: mol_type = other DNA
gcttggtgaggaccccaagctcggcactattgtgactgtgcttttggcttcctttccaggc

SEQ ID NO: 87 - DNA, 65 bp, Homo sapiens
  source 1..65: mol_type = other DNA
ctaggagggatgggagagaggactgtgaggcatgggtggctctatggtcacgcccatcttcctac

SEQ ID NO: 88 - DNA, 22 bp, Homo sapiens
  source 1..22: mol_type = other DNA
tagcagcacataatggtttgtg

SEQ ID NO: 89 - DNA, 22 bp, Mus musculus
  source 1..22: mol_type = other DNA
tagcagcacataatggtttgtg

SEQ ID NO: 90 - DNA, 22 bp, Homo sapiens
  source 1..22: mol_type = other DNA
tagcagcacatcatggtttaca

SEQ ID NO: 91 - DNA, 22 bp, Homo sapiens
  source 1..22: mol_type = other DNA
tagcagcacgtaaatattggcg

SEQ ID NO: 92 - DNA, 23 bp, Homo sapiens
  source 1..23: mol_type = other DNA
caaagtgcttacagtgcaggtag

SEQ ID NO: 93 - DNA, 23 bp, Homo sapiens
  source 1..23: mol_type = other DNA
actgccctaagtgctccttctgg

SEQ ID NO: 94 - DNA, 22 bp, Homo sapiens
  source 1..22: mol_type = other DNA
tagcttatcagactgatgttga

SEQ ID NO: 95 - DNA, 21 bp, Homo sapiens
  source 1..21: mol_type = other DNA
aggcggagacttgggcaattg

SEQ ID NO: 96 - DNA, 23 bp, Homo sapiens
  source 1..23: mol_type = other DNA
tagcaccatttgaaatcagtgtt

SEQ ID NO: 97 - DNA, 22 bp, Homo sapiens
  source 1..22: mol_type = other DNA
ctggtttcacatggtggcttag

SEQ ID NO: 98 - DNA, 23 bp, Homo sapiens
  source 1..23: mol_type = other DNA
tagcaccatttgaaatcagtgtt

SEQ ID NO: 99 - DNA, 21 bp, Homo sapiens
  source 1..21: mol_type = other DNA
cattattacttttggtacgcg

SEQ ID NO: 100 - DNA, 22 bp, Homo sapiens
  source 1..22: mol_type = other DNA
tcgtaccgtgagtaataatgcg

SEQ ID NO: 101 - DNA, 23 bp, Homo sapiens
  source 1..23: mol_type = other DNA
gtccagttttcccaggaatccct

SEQ ID NO: 102 - DNA, 22 bp, Homo sapiens
  source 1..22: mol_type = other DNA
ggattcctggaaatactgttct

SEQ ID NO: 103 - DNA, 21 bp, Homo sapiens
  source 1..21: mol_type = other DNA
tagcagcacagaaatattggc

SEQ ID NO: 104 - DNA, 23 bp, Mus musculus
  source 1..23: mol_type = other DNA
cagtgcaatggtattgtcaaagc

SEQ ID NO: 105 - DNA, 20 bp, Homo sapiens
  source 1..20: mol_type = other DNA
cctctgggcccttcctccag

SEQ ID NO: 106 - DNA, 22 bp, Homo sapiens
  source 1..22: mol_type = other DNA
tctctgggcctgtgtcttaggc

SEQ ID NO: 107 - DNA, 22 bp, Homo sapiens
  source 1..22: mol_type = other DNA
cagcagcaattcatgttttgaa

SEQ ID NO: 108 - DNA, 23 bp, Homo sapiens
  source 1..23: mol_type = other DNA
aatgacacgatcactcccgttga

SEQ ID NO: 109 - DNA, 22 bp, Homo sapiens
  source 1..22: mol_type = other DNA
agaggctggccgtgatgaattc

SEQ ID NO: 110 - DNA, 21 bp, Homo sapiens
  source 1..21: mol_type = other DNA
cagcagcacactgtggtttgt

SEQ ID NO: 111 - DNA, 23 bp, Homo sapiens
  source 1..23: mol_type = other DNA
caaagcgcttctctttagagtgt

SEQ ID NO: 112 - DNA, 22 bp, Homo sapiens
  source 1..22: mol_type = other DNA
aaagtgcttccctttggactgt

SEQ ID NO: 113 - DNA, 22 bp, Homo sapiens
  source 1..22: mol_type = other DNA
gaaggcgcttccctttagagcg

SEQ ID NO: 114 - DNA, 21 bp, Homo sapiens
  source 1..21: mol_type = other DNA
tgagttggccatctgagtgag

SEQ ID NO: 115 - DNA, 21 bp, Homo sapiens
  source 1..21: mol_type = other DNA
aggaggcagcgctctcaggac

SEQ ID NO: 116 - DNA, 21 bp, Homo sapiens
  source 1..21: mol_type = other DNA
gcaggaacttgtgagtctcct

SEQ ID NO: 117 - DNA, 21 bp, Homo sapiens
  source 1..21: mol_type = other DNA
tacttggaaaggcatcagttg

SEQ ID NO: 118 - DNA, 23 bp, Homo sapiens
  source 1..23: mol_type = other DNA
gcagcagagaataggactacgtc

SEQ ID NO: 119 - DNA, 21 bp, Homo sapiens
  source 1..21: mol_type = other DNA
gtgggcgggggcaggtgtgtg

SEQ ID NO: 120 - DNA, 21 bp, Homo sapiens
  source 1..21: mol_type = other DNA
tccttctgctccgtcccccag

SEQ ID NO: 121 - DNA, 24 bp, Homo sapiens
  source 1..24: mol_type = other DNA
agcctggaagctggagcctgcagt

SEQ ID NO: 122 - DNA, 17 bp, Homo sapiens
  source 1..17: mol_type = other DNA
gtgggggagaggctgtc

SEQ ID NO: 123 - DNA, 21 bp, Homo sapiens
  source 1..21: mol_type = other DNA
tgcaggaccaagatgagccct

SEQ ID NO: 124 - DNA, 22 bp, Homo sapiens
  source 1..22: mol_type = other DNA
agcagaggcagagaggctcagg

SEQ ID NO: 125 - DNA, 19 bp, Homo sapiens
  source 1..19: mol_type = other DNA
ggggcctggcggtgggcgg

SEQ ID NO: 126 - DNA, 22 bp, Homo sapiens
  source 1..22: mol_type = other DNA
tgcctggaacatagtagggact

SEQ ID NO: 127 - DNA, 22 bp, Homo sapiens
  source 1..22: mol_type = other DNA
attgcctctgttctaacacaag

SEQ ID NO: 128 - DNA, 22 bp, Homo sapiens
  source 1..22: mol_type = other DNA
tgtgttagaataggggcaataa

SEQ ID NO: 129 - DNA, 22 bp, Homo sapiens
  source 1..22: mol_type = other DNA
cggggagagaacgcagtgacgt

SEQ ID NO: 130 - DNA, 23 bp, Homo sapiens
  source 1..23: mol_type = other DNA
tggggacgtagctggccagacag

SEQ ID NO: 131 - DNA, 22 bp, Homo sapiens
  source 1..22: mol_type = other DNA
aggaggcatcttgagaaatgga

SEQ ID NO: 132 - DNA, 21 bp, Homo sapiens
  source 1..21: mol_type = other DNA
tgagtgttgtctacgagggca

SEQ ID NO: 133 - DNA, 22 bp, Homo sapiens
  source 1..22: mol_type = other DNA
ctgcagagtttgtacggaccgg

SEQ ID NO: 134 - DNA, 23 bp, Homo sapiens
  source 1..23: mol_type = other DNA
gcctggagctactccaccatctc

SEQ ID NO: 135 - DNA, 22 bp, Homo sapiens
  source 1..22: mol_type = other DNA
cagttgggtctaggggtcagga

SEQ ID NO: 136 - DNA, 17 bp, Homo sapiens
  source 1..17: mol_type = other DNA
ttggaggcgtgggtttt

SEQ ID NO: 137 - DNA, 17 bp, Homo sapiens
  source 1..17: mol_type = other DNA
gctgggcgaggctggca

SEQ ID NO: 138 - DNA, 18 bp, Homo sapiens
  source 1..18: mol_type = other DNA
agggggcgggctccggcg

SEQ ID NO: 139 - DNA, 18 bp, Homo sapiens
  source 1..18: mol_type = other DNA
aggctgggctgggacgga

SEQ ID NO: 140 - DNA, 22 bp, Homo sapiens
  source 1..22: mol_type = other DNA
caggatccacagagctagtcca

SEQ ID NO: 141 - DNA, 23 bp, Homo sapiens
  source 1..23: mol_type = other DNA
ctgggggacgcgtgagcgcgagc

SEQ ID NO: 142 - DNA, 22 bp, Homo sapiens
  source 1..22: mol_type = other DNA
agggggcgcagtcactgacgtg

SEQ ID NO: 143 - DNA, 25 bp, Homo sapiens
  source 1..25: mol_type = other DNA
agcggggaggaagtgggcgctgctt

SEQ ID NO: 144 - DNA, 23 bp, Homo sapiens
  source 1..23: mol_type = other DNA
ggcaggagggctgtgccaggttg

SEQ ID NO: 145 - DNA, 22 bp, Homo sapiens
  source 1..22: mol_type = other DNA
tgctgggggccacatgagtgtg

SEQ ID NO: 146 - DNA, 22 bp, Homo sapiens
  source 1..22: mol_type = other DNA
tgcggggacaggccagggcatc

SEQ ID NO: 147 - DNA, 21 bp, Homo sapiens
  source 1..21: mol_type = other DNA
tagcggggattccaatattgg

SEQ ID NO: 148 - DNA, 22 bp, Homo sapiens
  source 1..22: mol_type = other DNA
cagagtgacaagctggttaaag

SEQ ID NO: 149 - DNA, 20 bp, Homo sapiens
  source 1..20: mol_type = other DNA
gggctggggcgcggggaggt

SEQ ID NO: 150 - DNA, 21 bp, Homo sapiens
  source 1..21: mol_type = other DNA
tctattccccactctccccag

SEQ ID NO: 151 - DNA, 21 bp, Homo sapiens
  source 1..21: mol_type = other DNA
actgagcctctctctctccag

SEQ ID NO: 152 - DNA, 21 bp, Homo sapiens
  source 1..21: mol_type = other DNA
tctctggtctttccttgacag

SEQ ID NO: 153 - DNA, 20 bp, Homo sapiens
  source 1..20: mol_type = other DNA
tccactctcctggcccccag

SEQ ID NO: 154 - DNA, 22 bp, Homo sapiens
  source 1..22: mol_type = other DNA
tccctcgccttctcaccctcag

SEQ ID NO: 155 - DNA, 20 bp, Homo sapiens
  source 1..20: mol_type = other DNA
tctctctgactccatggcag

SEQ ID NO: 156 - DNA, 21 bp, Homo sapiens
  source 1..21: mol_type = other DNA
tctctggtcttgccaccccag

SEQ ID NO: 157 - DNA, 20 bp, Homo sapiens
  source 1..20: mol_type = other DNA
aagtcctgcttctgttgcag

SEQ ID NO: 158 - DNA, 21 bp, Homo sapiens
  source 1..21: mol_type = other DNA
tgaccccttctgtctccctag

SEQ ID NO: 159 - DNA, 19 bp, Homo sapiens
  source 1..19: mol_type = other DNA
gtggtctcttggcccccag

SEQ ID NO: 160 - DNA, 23 bp, Homo sapiens
  source 1..23: mol_type = other DNA
ttctctctgtctttctctctcag

SEQ ID NO: 161 - DNA, 23 bp, Homo sapiens
  source 1..23: mol_type = other DNA
cccatcacctttccgtctcccct

SEQ ID NO: 162 - DNA, 20 bp, Homo sapiens
  source 1..20: mol_type = other DNA
tctgtgcccctacttcccag

SEQ ID NO: 163 - DNA, 22 bp, Homo sapiens
  source 1..22: mol_type = other DNA
caagcctctcctgcccttccag

SEQ ID NO: 164 - DNA, 22 bp, Homo sapiens
  source 1..22: mol_type = other DNA
tctctctcccacttccctgcag

SEQ ID NO: 165 - DNA, 22 bp, Homo sapiens
  source 1..22: mol_type = other DNA
ttggtgaggaccccaagctcgg

SEQ ID NO: 166 - DNA, 21 bp, Homo sapiens
  source 1..21: mol_type = other DNA
tgggagagaggactgtgaggc

SEQ ID NO: 167 - DNA, 605 bp, Homo sapiens
  source 1..605: mol_type = other DNA
aagcgagctccagtccgctgtcaagatgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaat

SEQ ID NO: 168 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 169 - DNA, 547 bp, Mus musculus
  source 1..547: mol_type = other DNA
agactgagttccagactcccgtcaacatgttggtgacatacatccttgcctctgttttgctcttcagttctgtgctgggccagagatgcagcaccacatggggcatcagagacaccaattaccttattgaaaatctgaaggatgatccaccgtcaaaatgcagctgcagcggcaacgtgaccagctgcttgtgtctctccgtcccaactgatgattgtaccacaccgtgctacagggagggactgttacagctgaccaatgccacacagaaatcaagactcttgcctgttttccatcgggtgaaaaggatagttgaagtcctaaagaacatcacgtgtccgtccttttcctgcgaaaagccatgcaaccagaccatggcaggcaacacactgtcatttctgaagagtctcctggggacgttccagaagacagagatgcaaaggcagaaaagccgaccatgaagacagatgctatttattctatttattgaatttacaaaacctcccctccttaactgttacagtgaagaaataaactaagctattct

SEQ ID NO: 170 - protein, 144 aa, Mus musculus
MLVTYILASVLLFSSVLGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

(19) United States
(12) Patent Application Publication
(10) Pub. No.: US 2025/0283074 A1
(43) Pub. Date: Sep. 11, 2025

(54) CLUSTERIN OVEREXPRESSION IN ALZHEIMER’S DISEASE

(71) Applicant: University of Virginia Patent Foundation, Charlottesville, VA (US)

(72) Inventors: Alban Gaultier, Charlottesville, VA (US); Rebecca Beiter, Charlottesville, VA (US)

(73) Assignee: University of Virginia Patent Foundation, Charlottesville, VA (US)

(21) Appl. No.: 18/684,687

(22) PCT Filed: Aug. 17, 2022
(86) PCT No.: PCT/US2022/040649

§ 371 Date: Feb. 18, 2024

Related U.S. Application Data
(60) Provisional application No. 63/234,108, filed on Aug. 17, 2021.

(51) Int. Cl. C12N 15/113; A61K 48/00; C07K 14/54

(52) U.S. Cl. CPC C12N 15/113; A61K 48/0058; C07K 14/5425; C12N 2310/11; C12N 2310/531; C12N 2320/31

(57) ABSTRACT

Provided are methods for treating Alzheimer's Disease and/or ameliorating at least one symptom thereof. In some embodiments, the methods include administering to a. subject with AD an inhibitor of a clusterin biological activity, wherein the composition is administered via a route and in an amount sufficient to inhibit the clusterin biological activity to thereby treat the subject's. AD and/or ameliorate at least one symptom thereof. Also provided are methods for reducing and/or inhibiting myelin decay in a subject in need thereof and methods for inhibiting differentiation of oligodendrocyte progenitor cells (OPCs) to mature oligodendrocytes, the method comprising contacting the OPCs with a clusterin gene product or a functional fragment or derivative thereof.

### CROSS REFERENCE TO RELATED APPLICATION

The presently disclosed subject matter claims the benefit of U.S. Provisional Patent Application Ser. No. 63/234,108, filed Aug. 17, 2021, the disclosure of which incorporated herein by reference in its entirety.

### GOVERNMENT INTEREST

This invention was made with government support under Grant No. NS111204 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 160,418 byte UTF-8-encoded XML file created on Aug. 16, 2022 and entitled “3062_164_PCT.xml”. The Sequence Listing submitted via Patent Center is hereby incorporated by reference in its entirety.

A Substitute Sequence Listing XML has been electronically submitted to the United States Patent and Trademark Office via the Patent Center as a 161.228 byte UTF-8-encoded XML file created on Nov. 19, 2024 and entitled “Updated_3062_164_PCT_US.xml”. The Substitute Sequence Listing submitted via Patent Center is hereby incorporated by reference in its entirety.

### TECHNICAL FIELD

The presently disclosed subject matter relates to compositions and methods for diagnosing, preventing, and/or treating diseases, disorders, and condition associated with undesirable demyelination and/or ameliorating symptoms associated therewith, which in some embodiments relates to reducing and/or inhibiting myelin decay in a subject in need thereof. In some embodiments, the disease, disorder, or condition is selected from the group consisting of multiple sclerosis; spinal cord injury, brain injury, leukodystrophies, neuromyelitis optica (NMO), and Alzheimer's Disease (AD).

### BACKGROUND

Alzheimer's disease (AD) is a devastating neurodegenerative disease that impacts more than 6 million Americans. As the population ages, it is anticipated that the number of AD patients will drastically increase, furthering the impact of this disease on the health care system and care givers. Despite years of active research centered on understanding the role of Aβ and Tau, understanding of the disease remains incomplete and AD patients still lack effective therapeutic options that reduce disease symptomology and improve clinical outcomes.

In recent years, increasing amounts of data have pointed to myelin disruption as a significant pathological finding in Alzheimer's disease patients (Roher et al., 2002). Indeed, multiple studies have reported a decrease in myelinated axons as well as a decrease in total myelin proteins in both AD patients as well as in mouse models of AD (Roher et al., 2002, Desai et al., 2009; Desai et al., 2010; Zhan et al., 2014). Recent work has even put forward the concept that nmelin disruption is a key event that precipitates cognitive decline in AD (Bartzokis, 2011). While myelin loss has been documented as a key component of Alzheimer's, it remains unclear why oligodendrocyte progenitor cells (OPCs), a population able to produce new oligodendrocytes throughout adulthood, fail to compensate for the loss of myelin in the context of AD (Dimou et al., 2008; Rivers et al., 2008; Kang et al., 2010). Even more importantly, strategies aimed at promoting remyelination in order to delay the irreversible progression of neurodegeneration in AD have not been explored.

Clusterin, also known as apolipoprotein J, is a secreted chaperone protein that has been shown to have multiple functions including preventing apoptosis, inhibiting the complement cascade, and promoting clearance of cellular debris (Murphy et al., 1988; Falgarone & Chiocchia, 2009; Wyatt et al., 2011; Pereira et al., 2018). A very common SNP in CLU, present in 36% of the population, is the third risk factor for late onset AD and young healthy adults carrying the risk allele present with lower white matter integrity (Braskie et al., 2011). Clusterin has been shown to be increased in the brain of AD patients, as well as in the brains of AD mouse models (Hong et al., 2013; Miners et al., 2017) and increased levels of clusterin in the plasma of AD patients correlates with a more rapid cognitive decline. Importantly, carrying the risk factor SNP has been shown to elevate clusterin expression in the plasma of AD patients (Mullan et al., 2013). Despite evidence of a possible connection between clusterin, myelin, and AD, the role of clusterin in OPCs and myelination in the context of AD has never been studied.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter relates to methods for treating diseases, disorders, and/or conditions associated with undesirable demyelination and/or ameliorating at least one symptom associated therewith. In some embodiments, the methods comprise, consist essentially of, or consist of administering to a subject in need thereof a composition comprising, consisting essentially of, or consisting of an inhibitor of a clusterin biological activity. In some embodiments, the composition is administered via a route and in an amount sufficient to inhibit the clusterin biological activity to thereby treat the disease, disorder, or condition in the subject and/or to ameliorate at least one symptom thereof. In some embodiments, the disease, disorder, or condition is selected from the group consisting of multiple sclerosis; spinal cord injury, brain injury, leukodystrophies, neuromyelitis optica (NMO), and Alzheimer's Disease (AD). In some embodiments, the administering reduces an amount of clusterin in at least one cell type of the central nervous system (CNS) of the subject, optionally in the brain of the subject.

In some embodiments, the presently disclosed subject matter also relates to methods for reducing and/or inhibiting myelin decay in subjects in need thereof. In some embodiments, the methods comprise, consist essentially of, or consist of administering to the subject a composition comprising an inhibitor of a clusterin biological activity via a route and in an amount sufficient to reduce and/or inhibit myelin decay in the subject. In some embodiments, the subject has or is at risk for developing a disease, disorder, or condition, optionally a disease, disorder, or condition selected from the group consisting of multiple sclerosis, spinal cord injury, brain injury, leukodystrophies, neuromyelitis optica (NMO), and Alzheimer's Disease (AD).

In some embodiments of the presently disclosed methods, the inhibitor of a clusterin biological activity comprises an inhibitory nucleic acid that binds to and reduces translation of a clusterin gene product, optionally a human clusterin gene product. In some embodiments, the inhibitory nucleic acid targets a subsequence of a human clusterin gene product as set forth in Accession No. NM_001831.4 of the GENBANK® biosequence database (SEQ ID NO: 7).

In some embodiments of the presently disclosed methods, the inhibitor of a clusterin biological activity comprises a guide RNA (gRNA) that targets a clusterin gene product for modification with CRISPR/cas9. In some embodiments, the gRNA comprises a sequence that comprises, consists essentially of, or consists of a nucleotide sequence selected from the group consisting of CGTCTATGATGCTGGACGCG (SEQ ID NO: 2), GACGTACTTACTICCCTGAT (SEQ ID NO: 3), and GCGTGCGTAGAACTTCATGC (SEQ ID NO: 6) and/or that targets a clusterin gene product nucleotide sequence that comprises, consists essentially of, or consists of a nucleotide sequence selected from the group consisting of TACGCACGCGTCTGCAGAAG (SEQ ID NO: 1), AGAAGGCGACGATGAC (SEQ ID NO: 4), and CCGCCAACAGAATTCATACG (SEQ ID NO: 5).

In some embodiments, the presently disclosed subject matter relates to methods for inhibiting differentiation of oligodendrocyte progenitor cells (OPCs) to mature oligodendrocytes. In some embodiments, the methods comprise, consist essentially of, or consist of contacting the OPCs with a clusterin gene product or a functional fragment or derivative thereof. In some embodiments, the clusterin gene product comprises SEQ ID NO: 8 or a post-translationally modified subsequence thereof.

In some embodiments, the presently disclosed methods further comprise administering at least one additional therapy, optionally selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; a nucleotide reverse transcriptase inhibitor (NRTI), optionally an NRTI abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof; a non-nucleoside reverse transcriptase inhibitor (NNRTI), optionally an NNRTI selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof. In some embodiments, the at least one additional therapy comprises treatment with an acetylcholinesterase (AChE) inhibitor. In some embodiments, the AChE inhibitor is selected from the group consisting of donepezil, rivastigmine, and galantamine. In some embodiments, the at least one additional therapy comprises treatment with an N-methyl-d-aspartate receptor (NMDAR) antagonist. In some embodiments, the NMDAR antagonist is memantine. In some embodiments, the at least one additional therapy comprises treatment with a secretase inhibitor. In some embodiments, the at least one additional therapy comprises treatment with a beta-site APP-cleaving enzyme (BACE) inhibitor. In some embodiments, the at least one additional therapy comprises treatment with an inhibitor of tau aggregation. In some embodiments, the at least one additional therapy comprises treatment with an inhibitory nucleic acid. In some embodiments, the miRNA is an miRNA selected from the group consisting of miR-126, miR-145, miR-195, miR-21, and miR-29b. In some embodiments, the at least one additional therapy comprises treatment with a nucleotide reverse transcriptase inhibitor (NRTI). In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, and pharmaceutically acceptable salts thereof. In some embodiments, the NRTI derivative is an alkylated NRTI derivative. In some embodiments, the alkylated NRTI derivative is tri-methoxy-3TC. In some embodiments, the at least one additional therapy comprises treatment with a non-nucleoside reverse transcriptase inhibitor (NNRTI). In some embodiments, the NNRTI is selected from the group consisting of delavirdine (DLV), efavirenz (EFV), etravirine (ETR), nevirapine (NVP), rilpivirine (TMC278), doravirine (MK-1439), derivatives thereof, and pharmaceutically acceptable salts thereof.

In some embodiments, the presently disclosed methods further comprise administering to a subject in need thereof an additional composition, wherein the administering is in an amount and via a route sufficient to induce an interleukin 9 (IL-9) biological activity in the subject. In some embodiments, the administering is within the nervous system of the subject in need thereof. In some embodiments, the additional composition comprises, consists essentially of, or consists of a biologically active IL-9 polypeptide, a biologically active fragment thereof, a vector encoding a biologically active IL-9 polypeptide and/or a biologically active fragment thereof, optionally a viral vector, further optionally an adeno-associated virus (AAV) vector, a small molecule that induces IL-9 biological activity, an IL-9 receptor agonist, and/or a genetic construct that induces IL-9 biological activity in the subject in need thereof.

The presently disclosed subject matter also relates in some embodiments to methods for treating diseases, disorders, and/or conditions associated with undesirable demyelination and/or ameliorating at least one symptom thereof, wherein the methods comprise, consist essentially of, or consist of administering to a subject with a disease, disorder, or condition associated with undesirable demyelination one or more compositions that individually or together comprise, consist essentially of, or consist of an inhibitor of a clusterin biological activity and/or an inducer of IL-9 biological activity, wherein the at least one composition is administered via a route and in an amount sufficient to inhibit clusterin biological activity and/or induce IL-9 biological activity in the subject to thereby treat the subject's disease, disorder, or condition and/or to ameliorate at least one symptom thereof. In some embodiments, the disease, disorder, or condition is selected from the group consisting of multiple sclerosis: spinal cord injury, brain injury, leukodystrophies, neuromyelitis optica (NMO), and Alzheimer's Disease (AD). In some embodiments, the administering reduces an amount of clusterin and/or increases an amount of IL-9 in at least one cell type of the central nervous system (CNS) of the subject, optionally in the brain of the subject. In some embodiments, the inducer of IL-9 biological activity comprises, consists essentially of, or consists of a biologically active IL-9 polypeptide, a biologically active fragment thereof, a vector encoding a biologically active IL-9 polypeptide and/or a biologically active fragment thereof, optionally a viral vector, further optionally an adeno-associated virus (AAV) vector, a small molecule that induces IL-9 biological activity, an IL-9 receptor agonist, and/or a genetic construct that induces IL-9 biological activity in the subject.

The presently disclosed subject matter also relates in some embodiments to methods for reducing and/or inhibiting myelin decay in a subject in need thereof, wherein the methods comprise, consist essentially of, or consist of administering to the subject at least one composition comprising, consisting essentially of, or consisting of an inhibitor of a clusterin biological activity and/or an inducer of IL-9 biological activity, wherein the at least one composition is administered via a route and in an amount sufficient to inhibit clusterin biological activity and/or induce IL-9 biological activity in the subject to thereby reduce and/or inhibit myelin decay in the subject. In some embodiments, the subject has or is at risk for developing disease, disorder, or condition selected from the group consisting of multiple sclerosis; spinal cord injury, brain injury, leukodystrophies, neuromyelitis optica (NMO), and Alzheimer's Disease (AD), or a worsening of symptoms associated therewith. In some embodiments, the inhibitor of a clusterin biological activity comprises an inhibitory nucleic acid that binds to and reduces translation of a clusterin gene product, optionally a human clusterin gene product. In some embodiments, the inhibitory nucleic acid targets a subsequence of a human clusterin gene product as set forth in Accession No. NM_001831.4 of the GENBANK® biosequence database (SEQ ID NO: 7). In some embodiments, the inhibitor of a clusterin biological activity comprises a guide RNA (gRNA) that targets a clusterin gene product for modification with CRISPR/cas9. In some embodiments, the gRNA comprises a sequence that comprises, consists essentially of, or consists of a nucleotide sequence selected from the group consisting of CGTCTATGATGCTGGACGCG (SEQ ID NO: 2), GACGTACTTACTTCCCTGAT (SEQ ID NO: 3), and GCGTGCGTAGAACTTCATGC (SEQ ID NO: 6) and/or that targets a clusterin gene product nucleotide sequence that comprises, consists essentially of, or consists of a nucleotide sequence selected from the group consisting of TACGCACGCGTCTGCAGAAG (SEQ ID NO: 1), AGAAGGCGACGATGAC (SEQ ID NO: 4), and CCGCCAACAGAATTCATACG (SEQ ID NO: 5). In some embodiments, the inducer of IL-9 biological activity comprises, consists essentially of, or consists of a biologically active IL-9 polypeptide, a biologically active fragment thereof, a vector encoding a biologically active IL-9 polypeptide and/or a biologically active fragment thereof, a small molecule that induces IL-9 biological activity, an IL-9 receptor agonist, and/or a genetic construct that induces IL-9 biological activity in the subject. In some embodiments, the vector is a viral vector, which in some embodiments can be an adeno-associated virus (AAV) vector,

Accordingly, it is an object of the presently disclosed subject matter to provide compositions and methods for inhibiting undesirable clusterin biological activities, including but not limited to undesirable clusterin biological activities associated with diseases, disorders, and/or conditions such as Alzheimer's Disease.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Description, Figures, and EXAMPLES.

### BRIEF DESCRIPTION OF THE FIGURES

The patent or application file contains at least one Figure executed in color. Copies of this patent or patent application publication with color Figure(s) will be provided by the Office upon request and payment of the necessary fee.

FIGS. 1A and 1B: Model to Label adult OPCs. FIG. 1A is a representative image of YFP labeled OPCs (PDGFRα+Olig2+) cells following 2 tamoxifen injections. Arrows indicate YFP+ OPCs, arrowhead indicates YFP-OPCs. Scale bar is 30 μM. FIG. 1B is a graph of percent of OPCs expressing YFP following 0, 1, 2, or 3 tamoxifen injections. Each injection was given 3 days apart. n=2-3 mice/group.

FIGS. 2A-2D: Two distinct clusters of OPCs in the adult brain. FIG. 2A is a tSNE plot of all sequenced cells isolated from PDGFRα-CreER reporter brains. Clusters were labeled with cell-type classifications based on expression of common cell-type markers. N=3 independent experiments, n=6 samples. FIGS. 2B and 2C are a series of violin plots depicting expression of common OPC markers in each cluster. Each dot represents a cell. Expression value is plotted on the y-axis. FIG. 2D is a heatmap depicting the scaled and log-normalized expression values of the top 10 most highly enriched genes in each cluster. PC=Pericytes.

FIGS. 3A-3C: Adult OPCs express Clu and Gpr17. FIG. 3A is a graph of cell-specific expression of markers used for cluster validation including Clu (OPC1) and Gpr17 (OPC2) overlaid on a tSNE map. FIG. 3B is an RNAscope expression of Pdgfra (red), Olig1 (green), Clu (white), and Hoechst (blue). FIG. 3C is a graph of the quantification of the percentage of OPCs that are Clu negative (<10 puncta) and Clu positive (>10 puncta). N=3 individual experiments, n=4 samples, with a total of 247 OPCs quantified. Each sample includes quantification of marker expression from the cortex, hippocampus, corpus callosum, and cerebellum. Scale bar=10 μm.

FIGS. 4A and 4B: Adult OPCs do not express markers of multiple clusters. FIG. 4A is a series of tepresentative PrimeFlow gatings of brain cells stained for CD45 protein and Clu, Gpr17, Pdgfra, and Olig2 RNA. FIG. 4B is a graph of the quantification of Live/CD45−/Olig2+ cells that express Clu alone, Gpr17 alone, or both Clu and Gpr17. N=2 individual experiments, n=9 samples. Analyzed using one-way repeated measures ANOVA. ****p<0.0001, *p<0.05.

FIGS. 5A-5E: Clusterin is upregulated in Alzheimer's Disease and 5×FAD brains. FIG. 5A is a photomicrograph showing clusterin protein expression (IHC, brown) in the cortex of a normal aging brain and a late-stage AD brain. FIG. 5B is a photomicrograph showing CLU RNA expression (ISH, red) and As protein expression (IHC, brown) in a late-stage AD brain (n=1). FIG. 5C is a photomicrograph showing CLU RNA (ISH, red) and clusterin protein (IHC, gray) in late-stage AD brain. FIG. 5D is a photomicrograph showing clusterin protein expression (white) in the cortex of 6-month old 5×FAD mice. FIG. 5E is a graph of the quantification of clusterin staining depicted in panel D. N=1 independent experiment, n=3 per group. Data analyzed using an unpaired t-test. *p<0.05. Error bars represent standard error of the mean.

FIGS. 6A-6D: Clusterin inhibits OPC differentiation. FIG. 6A is a series of bar graphs showing expression of Mbp, Pip1, Cnp, and Myrf, measured by qPCR in cells cultured in proliferation media (OPC_Veh), differentiation media (OLG_Veh), or differentiation media supplemented with 8 μg/ml of clusterin (OLG_Clu). Data was analyzed using a mixed-effects analysis followed by a Tukey's multiple comparisons test. N=3 individual experiments, n=7 samples per group. FIG. 6B is a series of representative fluorescence micrograph images of cells cultured in differentiation media (Vehicle) or differentiation media supplemented with 8 μg/ml of clusterin (Clusterin). Oligodendrocytes are stained using MBP (white), Olig2 (red), and Dapi (blue). FIG. 6C is a bar graph of the quantification of the differentiation efficiency of cells depicted in FIG. 6B. Oligodendrocytes were identified as expressing MBP and Olig2, and data was normalized to the total number of Olig2+ cells per sample. N=2 independent experiments, n=5 samples per group. FIG. 6D is a bar graph showing the results of CCK8 assays for OPC treated with vehicle (OPC_veh) or 8 μg/mL clusterin (OPC_Clu). Data was is analyzed using a repeated measures t-test. N=4 individual experiments, n=11 samples per group *p<0.05, **p<0.01, ****p<0.0001. Error bars represent SEM.

FIGS. 7A-7F: Macromolecules and protein aggregates cause OPCs to upregulate clusterin. OPCs treated with 10 ng/ml TNFα for 3 hours (FIG. 7A), 10 μM H₂O₂ for 3 hours (FIG. 7B), 3 μM As for 2 hours (FIG. 7C), a 1:1 ratio of apoptotic cells for 6 hours (FIG. 7D), 100 μg/ml myelin debris for 4 hours (FIG. 7E), or 100 μg/ml myelin debris and 1 μM Cytochalasin D for 4 hours (FIG. 7F). Clusterin transcript quantity was measured using qPCR. N=1-2 independent experiments. n=4-8 samples/condition. All data was analyzed using a repeated measures t-test. ns: not significant; CytoD: Cytochalasin D. *p<0.05, **p<0.01.

FIGS. 8A and 8B: Aβ injections result in clusterin upregulation. FIG. 8A depicts clusterin (red) expression following injection of CyPHer-labeled Aβ (white). Contralateral hemisphere used as uninjected control. Scale bar=100 μM. FIG. 8B is a series of fluorescence micrographs showing OPCs expressing PDGFRα (green) and Olig2 (red) engulf Aβ (white) following injection. Arrows indicate Aβ puncta inside an OPC cell body. Scale bar=20 μm.

FIGS. 9A and 9B: Cuprizone-induced demyelination results in increase clusterin expression in the corpus callosum. FIG. 9A is a photomicrograph showing clusterin expression (white) in the corpus callosum of a naïve mouse and a mouse following 5-weeks of cuprizone diet followed by 1-week of normal chow to allow remyelination to begin. FIG. 9B is a bar graph showing quantification of clusterin coverage of the corpus callosum depicted in FIG. 9A. N=1 independent experiment, n=5 mice per group. Data analyzed using an unpaired t-test. ***p<0.001. Error bars represent standard error of the mean.

FIG. 10: Clusterin RNA expression in OPCs from a late-stage AD brain. RNAscope staining of an OPC (expressing Pdgfra in green and Olig2 in white) that expresses CLU (red) in the gray matter of a patient with late-stage AD. Gray arrowhead indicates a CLU+ OPC. White dotted lines indicate nuclei.

FIGS. 11A-11J: A subset of OPCs expresses the AD-risk factor clusterin. Representative images (FIG. 11A) and quantification (FIG. 11B) of clusterin expression (immunohistochemistry, brown) in the cortex from a normal aging patient (n=15) and a late-stage AD patient (n=26). FIG. 11C shows detection of clusterin RNA (in situ hybridization, red) and Aβ protein (immunohistochemistry, brown) in late-stage AD brain (late-stage AD n=2). Representative images (FIG. 11D) and quantification (FIG. 11E) of clusterin expression in the cortex and hippocampus of WT and 5×FAD mice. (n=6; Scale bar=30 μm). Representative images (FIG. 11F) and quantification (FIG. 11G) of in situ hybridization for OPCs (Pdgfra in green, Olig1 in white) expressing or lacking clusterin (Clu; red). n=4 with 206 cells; Scale bar=10 μm. Representative gating strategy (FIG. 11H) and quantification (FIG. 11I) for RNA flow cytometry (n=9). FIG. 11J. In situ hybridization for OPCs (PDGFRA in green, OLIG2 in white) expressing clusterin (CLU; red) in normal aging and late-stage AD brains (Scale bar=10 μm).

FIGS. 12A-12I: Phagocytosis of extracellular debris drives clusterin expression in OPCs. FIG. 12A. Representative images of OPCs (PDGFRα in green, Olig2 in red) surrounding Aβ plaques (n=4; Scale bar=20 μm). FIG. 12B. Representative orthogonal view of CypHer-labeled Aβ (white) inside an OPC (PDGFRα in green. Olig2 in red) following injection of Aβ (n=6; Scale bar=10 μm). Representative images (FIG. 12C) and quantification (FIG. 12D) of clusterin expression (red) in the ipsilateral (CypHer-Aβ injected: green) and contralateral (FITC injected; green) hemispheres following injection (n=6 mice: Scale bar=100 μm) FIG. 12E. qPCR analysis of clusterin expression in OPCs following a 4-hour in vitro treatment with 3 μM Aβ and the phagocytosis blocker CytoD (1 μM) or vehicle control (n=24). f, Quantification of clusterin protein in OPCs following 72-hour treatment with 3 μM Aβ or vehicle control (n=10). qPCR analysis of clusterin expression in OPCs following a 4-hour in vitro treatment with 100 μg/ml myelin (FIG. 12G), cytokines (10 ng/ml TNFα or 10 ng/ml IFNγ; FIG. 12H) or 10 μM H₂O₂ (FIG. 12I) and CytoD (1 μM) or vehicle control (n=4-19).

FIGS. 13A-13G: Exogenous clusterin inhibits OPC differentiation. Expression of Mbp (FIG. 13A), Plp1 (FIG. 13B), Cnp (FIG. 13C), and Myrf (FIG. 13D), measured by qPCR in OPCs or OLGs treated with vehicle or 8 μg/ml of clusterin for 72 hours (n=7). Representative images (FIG. 13E) and quantification (FIG. 13F) of MBP expression in cells treated with or without 8 μg/ml of clusterin for 72 hours (n=5: Scale bar=50 μm). FIG. 13G. Quantification of OPCs viability following a 72 hours incubation with or without 8 μg/ml clusterin (n=11).

FIGS. 14A-14D: Clusterin inhibits differentiation by blocking IL-9 production. FIG. 14A. Quantification of cytokines present in the supernatant from OPCs treated with 8 μg/mL clusterin or vehicle control (n=6). Expression of Mbp (FIG. 14B), Plp1 (FIG. 14C), and Myrf (FIG. 14D), measured by qPCR in OPCs cultured in proliferation media (OPC Vehicle), differentiation media supplemented with vehicle (OLG Vehicle), or 8 μg/ml of is clusterin (OLG CLU), or 100 ng/ml IL-9 (OLG IL-9), or 8 μg/ml of clusterin and 100 ng/ml IL-9 (OLG CLU+IL-9) for 72 hours (n=11).

### BRIEF DESCRIPTION OF THE SEQUENCE LISTING

SEQ ID NOs: 1-6 are subsequences of the exemplary human clusterin (CLU) gene product of SEQ ID NO: 7 that serve as exemplary, non-limiting targets or targeting sequences for inhibitory nucleic acids directed to human CLU gene products.

SEQ ID NO: 7 is a nucleic acid sequence of an exemplary human CLU gene product as disclosed in Accession No. NM_001831.4 of the GENBANK® biosequence database. The coding sequence of SEQ ID NO: 7 corresponds to nucleotides 76-1605 of SEQ ID NO: 7 and encodes a protein with an amino acid sequence as set forth in Accession No. NP_001822.3 of the GENBANK® biosequence database, which is SEQ ID NO: 8.

SEQ ID NO: 9 is a nucleic acid sequence of an exemplary murine CLU gene product as disclosed in Accession No. NM_013492.3 of the GENBANK® biosequence database. The coding sequence of SEQ ID NO: 9 corresponds to nucleotides 24-1580 of SEQ ID NO: 9 and encodes a protein with an amino acid sequence as set forth in Accession No. NP_038520.2 of the GENBANK® biosequence database, which is SEQ ID NO: 10.

SEQ ID NO: 11 is the nucleotide sequence of OGX-011, an antisense nucleic acid that targets clusterin.

SEQ ID NOs: 12-87 are the nucleotide sequences of exemplary pre-miRNAs of the presently disclosed subject matter.

SEQ ID NOs: 88-166 are the nucleotide sequences of exemplary mature miRNAs of the presently disclosed subject matter.

SEQ ID NO; 167 is a nucleic acid sequence of an exemplary human IL-9 gene product as disclosed in Accession No. NM_000590.2 (Homo sapiens interleukin 9 (IL9), mRNA) of the GENBANK® biosequence database. The coding sequence of SEQ ID NO: 167 corresponds to nucleotides 26-460 of SEQ ID NO: 167 and encodes a protein with an amino acid sequence as set forth in Accession No. NP_000581.1 of the GENBANK® biosequence database, which is SEQ ID NO: 168.

SEQ ID NO: 169 is a nucleic acid sequence of an exemplary murine IL-9 gene product as disclosed in Accession No. NM_008373.2 of the GENBANK) biosequence database. The coding sequence of SEQ ID NO: 169 corresponds to nucleotides 27-461 and encodes a protein with an amino acid sequence as set forth in Accession No. NP_032399.1 of the GENBANK® biosequence database, which is SEQ ID NO: 170.

### DETAILED DESCRIPTION

Alzheimer's disease (AD) is a devastating neurodegenerative disease that impacts more than 6 million Americans. Despite years of active research centered on understanding the role of Aβ and Tau, our understanding of the disease remains incomplete and the AD patient population remains without effective therapeutic options to tackle disease symptomology.

In recent years, increasing amounts of data have pointed to myelin disruption as a significant pathological finding in Alzheimer's disease patients. Recent work has even put forward the concept that myelin disruption is a key event that precipitates cognitive decline in AD. It remains unclear why oligodendrocyte progenitor cells (OPCs), a population able to produce new oligodendrocytes throughout adulthood, fails to repair myelin in the context of AD.

The pathophysiology of AD has been the focus of an immense amount of scientific research. While the deposition of beta-amyloid plaques (Aβ plaques) and the presence of dystrophic neurites have been thoroughly documented, the mechanism through which these Aβ plaques result in neuronal death and severe cognitive impairment remains unclear (Zhan et al., 1995, Maurice & Su. 2009). Interestingly, although numerous clinical trials of anti-amyloid therapeutics have successfully decreased the Aβ plaque-burden in Alzheimer's patients, none of them have successfully reduced clinical symptoms of cognitive impairment (Huang et al., 2020). Our proposal is significant because it focuses on myelin and the oligodendrocytes lineage which, despite being impacted during AD pathology, is vastly understudied and could be explored as a therapeutic option for AD.

In recent years, increasing amounts of data have pointed to white matter loss and myelin disruption as a significant pathological finding in Alzheimer's disease patients (Roher et al., 2002; Kurihara et al., 2010). Multiple studies have reported a decrease in myelinated axons as well as a decrease in total myelin proteins in both AD patients as well as in mouse models of AD (Roher et al., 2002; Desai et al., 2009; Desai et al., 2010; Zhan et al., 2014). Recent work has even postulated that myelin disruption is a key event that precipitates cognitive decline in AD (Bartzokis, 2011). In support of this concept, defects in white matter tracts are detected in preclinical AD patients, suggesting that myelin loss is not dependent on neurodegeneration, but instead may precede it (Dean et al., 2017). While myelin loss has been recognized as a key component of Alzheimer's, it remains unclear why oligodendrocyte progenitor cells (OPCs), a population which remains mitotically active and able to produce new oligodendrocytes throughout adulthood, fails to compensate for the loss of myelin in the context of AD (Dimou et al., 2008; Rivers et al., 2008; Kang et al., 2010).

In comparison to neurons and other glial subtypes, relatively little is known about how OPCs are affected by AD progression. Recent work has demonstrated a decrease in OPC density in AD mouse models, accompanied by significant changes in cell volume and morphology (Chacon-De-La-Rocha et al., 2020; Vanzulli et al., 2020). Additionally, OPCs have been implicated as important mediators of memory deficits in AD. In 2019, Zhang and colleagues demonstrated that senescent OPCs cluster around Aβ plaques in human AD brains and that a regiment of senolytic drugs not only reduces that population of senescent OPCs found in mice with Aβ plaques, but also improves the memory deficits normally observed in these mice (Zhang et al., 2019). Additionally, a recent paper demonstrated that the pro-myelinating therapeutic Clemastine improved both myelin integrity and memory performance in the APP/PS1 model of AD (Chen et al., 2021b). These data support recent evidence indicating that the generation of new myelin is critical for memory consolidation and preservation (Pan et al., 2020; Steadman et al., 2020; Xin & Chan, 2020). Taken together, this recent data indicates that OPCs and myelination are important factors in AD, although how they affect disease progression remains unclear. Our proposal aims to understand why myelin may be lost in AD and the value of sparing myelin in animal models of the disease aging samples. Given recent studies showing that new myelin formation in adult is critical for memory formation, memory recall and that promoting myelin repair is beneficial in preclinical model of AD (Pan et al., 2020; Steadman et al., 2020; Xin & Chan, 2020; Chen et al., 2021b), our project is significant.

While the precise etiology of AD remains elusive, genome-wide association studies (GWAS) has discovered important risk factors for Late Onset Alzheimer's disease (LOAD). Relevant to this proposal, a SNP in clusterin, called rs1 I136000, is a very common risk factor for LOAD expressed by 36% of the population (DeMattos et al., 2001; Bertram et al., 2007; Bertram & Tanzi, 2009; Harold et al., 2009). Clusterin (CLU), also known as apolipoprotein J, is a secreted chaperone protein that has been shown to have multiple functions in different cell types, including preventing apoptosis, inhibiting the complement cascade, and promoting clearance of cellular debris (Murphy et al., 1988; Falgarone & Chiocchia, 2009; Wyatt et al., 2011; Pereira et al., 2018). Clusterin has been shown to be increased in the brain, plasma, and cerebrospinal fluid of AD patients, as well as in the brains of AD mouse models (Hong et al., 2013; Miners et al., 2017). The role of clusterin in disease progression is unclear, but increased levels of clusterin in the plasma of AD patients correlates with a more rapid cognitive decline and brain atrophy, and clusterin expression in the brains of AD mice positively correlates with Aβ load (Thambisetty et al., 2010). A role clusterin in AD is also supported by studies of clusterin deletion in mouse models of AD, which results in reduced plaque load and improved performance of memory tasks. There are, however, reports that suggest that clusterin might be involved in clearance of Aβ plaques and protection of neurons (Wojtas et al., 2020; Chen et al., 2021a).

Importantly, carrying the SNP rs11136000 has been shown to elevate clusterin expression in the plasma of AD patients with this SNP (Mullan et al., 2013). Furthermore, young healthy adults carrying the clusterin risk allele present with lower white matter integrity as measured by Diffusion Tensor Imaging (Braskie et al., 2011). Additionally, data obtained with the amyloid precursor protein/presenilin 1 (APP/PS1) mouse model of AD shows that lack of clusterin leads to a decreased parenchymal plaque load, but an increase in cerebral amyloid angiopathy (Wojtas et al., 2017). However, additional evidence implies that astrocyte-derived clusterin may be protective by preventing plaques formation and improving synapse function in a mouse model of AD, indicating that clusterin is a mutli-functional protein that is likely beneficial at proper levels, but may become detrimental in situations of significant upregulation (Wojtas et al., 2020; Chen et al., 2021a). This data indicates that clusterin likely plays a significant role in the pathophysiology of Alzheimer's disease, although an appreciable amount of work remains to detail its exact mechanisms of action in disease progression. In this application, we propose to explore the pathological role of clusterin on myelin using an animal model of AD.

Exciting studies have provided compelling evidence that OPCs are capable of more than generating mature oligodendrocytes (Fernandez-Castaneda & Gaultier, 2016). OPCs have been shown to modulate neuronal and astrocytic functions, with direct behavioral consequence (Sakry et al., 2014; Birey et al., 2015). For example, depletion of OPCs in the prefrontal cortex has been shown to alter glutamatergic signaling and promote depressive-like behavior in mice (Birey et al., 2015). Because adult OPCs are evenly distributed throughout the CNS, they are ideally placed to detect perturbations. OPCs rapidly respond to CNS injury and disease by proliferation and repopulation of the lesion site (Simon et al., 2011; Kang et al., 2013b). In a mouse model of cerebral prolonged hypoperfusion, OPCs are the initial producers of MMP9, an enzyme responsible for the opening of the BBB-OPCs therefore can control the infiltration of neutrophils that ultimately damage the myelin sheath in this particular model (Seo et al., 2013). Recently, OPCs have been shown to contribute to the neuroinflammatory response. For is example, OPCs isolated from the brain of mice undergoing cuprizone-induced demyelination express high levels of CCL-2 and IL-10 (Moyon et al., 2015), two key immune mediators (Deshmane et al., 2009; Sims & Smith, 2010). OPCs can also present antigen to the adaptive immune system and directly modulate the course of the disease in animal models of multiple sclerosis (Falcao et al., 2018; Kirby et al., 2019; Fernandez-Castaneda et al., 2020). Relevant to this proposal, a recent nuclear sequencing study has revealed that human OPCs express and upregulate clusterin in AD (Grubman et al., 2019). While there is ample suggestion in the literature that adult OPCs can execute other tasks beside myelination, the role of OPCs in AD pathology remains unexplored.

Published literature thus supports the concept that myelin pathology is present in AD and in preclinical models of AD (Roher et al., 2002; Desai et al., 2009; Desai et al., 2010; Zhan et al., 2014). More importantly, a recent publication demonstrates that boosting myelin repair is beneficial in preclinical AD models (Chen et al., 2021b). Furthermore, the role of clusterin as an AD risk factor has been reported extensively, but the exact mechanism of action remains unclear (DeMattos et al., 2001; Bertram et al., 2007; Bertram & Tanzi. 2009; Harold et al., 2009). Our preliminary data demonstrates that OPCs express clusterin in the adult CNS, confirming recent human data (Grubman et al., 2019). We also document a strong upregulation of clusterin in the 5×FAD model that can be recapitulated in vitro by treating OPCs with oligomeric Aβ. Ultimately, our exciting data show that exogenous clusterin can block OPC differentiation into oligodendrocytes. Taken together, these results form a strong premise to support the hypothesis that clusterin acts as an inhibitor of myelin repair preventing OPCs from differentiating into oligodendrocytes in AD. The presently disclosed subject matter thus relates in some embodiments to examining OPC behavior in AD and identifying ways in which clusterin might direct OPCs away from differentiation and towards other functions in the context of pathology.

Thus, disclosed herein are investigations into the biological activities of clusterin, a protein that is highly expressed in the brain of Alzheimer's Disease patients and that has been identified as a significant risk factor this condition. As set forth herein, clusterin overexpression could be responsible for the myelin decay observed in Alzheimer's Disease. Thus, modulating the biological activities of clusterin can be employed to promote neuroprotection and delay the irreversible cognitive decline currently facing Alzheimer's Disease patients.

Support for this notion comes from the discovery that OPCs express clusterin in AD and a model of AD as well as from preliminary work showing that clusterin affects OPCs differentiation into oligodendrocytes.

Thus, whether clusterin can act as an inhibitor of myelin repair by directing OPCs away from differentiation into oligodendrocytes is addressed by pursuing three specific avenues:
1: Understanding the mechanisms regulating clusterin expression and its inhibition of OPC differentiation:
2: Assessing the impact of clusterin on oligodendrocytes and myelination in an animal model of Alzheimer's Disease; and
3: Quantifying the number of OPCs and Oligodendrocytes in normal aging vs AD patients.

With respect to the first avenue, the factors that drive expression of clusterin in OPCs and the signaling pathways involved in clusterin's impact on OPC differentiation are investigated.

In the second avenue, genetic and therapeutic inhibition of clusterin are employed to understand how clusterin impacts myelin repair in a preclinical model of AD.

The third avenue involves precisely quantifying OPCs, oligodendrocytes, and clusterin expression in normal aging and AD patients.

The presently disclosed subject matter thus relates to the roles of clusterin and myelin in AD pathology, an avenue that is anticipated lead to new treatments for AD. The presently disclosed subject matter is significant because these studies provide new knowledge to the community about the contribution of oligodendrocyte to AD pathology, facilitating the development of compositions and methods for treating and/or preventing the development of AD and/or inhibiting the development and/or progression of at least one symptom thereof.

An overall goal is to understand the function of clusterin on OPCs, oligodendrocytes and myelin in the context of AD. First, the mechanism by which clusterin affects differentiation of OPCs in vitro is investigated. Additionally, the impact of modulating clusterin expression (genetically and pharmacologically) on the progression of pathology in the 5×FAD animal model of AD is tested. Furthermore, using a combination of RNA scope and immunohistochemistry, the numbers of OPCs and oligodendrocytes in normal aging and AD brains are examined, and the fraction of OPCs that express clusterin in both conditions are quantified.

### I. DEFINITIONS

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in Table T.

TABLE 1
Amino Acids, Codes, and Functionally Equivalent Codons

| | 3-Letter | 1-Letter | |
|---|---|---|---|
| Full Name | Code | Code | Functionally Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other is chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl. L-3,4-dihydroxyphenylalanyl, alpha-amino acids such as L-alpha-hydroxylysyl and D-alpha-methylalanyl. L-alpha-methylalanyl, beta-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
I. Small aliphatic, nonpolar, or slightly polar residues. Ala, Ser, Thr. Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv. Fab, and F(ab)₂, as well as single chain antibodies and humanized antibodies. The antibodies that can be employed in the compositions and methods of the presently disclosed subject matter may exist in a variety of forms including, for example, polyclonal antibodies, monoclonal antibodies, Fv, Fab and F(ab)₂, as well as single chain antibodies and humanized antibodies (Bird et al., 1988; Huston et al., 1988; Harlow et al., 1989; Harlow et al., 1999).

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specify ing the antibody, wherein the DNA or amino acid sequence has been obtained using synthetic DNA or amino acid sequence technology which is available and well known in the art.

As used herein, the term “single chain variable fragment” (scFv) refers to a single chain is antibody fragment comprised of a heavy and light chain linked by a peptide linker. In some cases scFv are expressed on the surface of an engineered cell, for the purpose of selecting particular scFv that bind to an antigen of interest.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. The term “immunogen” is used interchangeably with “antigen” herein.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat, urine, and cerebrospinal fluid.

As used herein, the term “clusterin” refers to a gene that encodes a secreted chaperone protein and its transcription and translation products. The human clusterin (CLU) gene is located on chromosome 8 and corresponds to the reverse complement of nucleotides 27,596,917-27,614,700 of Accession No. NC_000008.11 of the GENBANK® biosequence database. Exemplary human CLU gene products include transcription products discloses as Accession No. NM_001831.4 (Homo sapiens clusterin (CLU), transcript variant 1, mRNA; SEQ ID NO: 7) of the GENBANKK biosequence database, which encodes the clusterin preproprotein of Accession No. NP_001822.3 of the GENBANK® biosequence database (SEQ ID NO: 8). Orthologs of clusterin from non-human species include Accession Nos. NM_013492.3 and NP_038520.2 (mouse; SEQ ID NOs: 9 and 10, respectively), NM_053021.2 and NP_444180.2 (rat), NM_204900.1 and NP_990231.1 (chicken), XM_519677.2 and XP_519677.2 (chimpanzee), NM_001003370.1 and NP_001003370.1 (dog), and NM_173902.2 and NP_776327.1 (cow) of the GENBANK® biosequence database, among others.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, in some embodiments at least about 50%, and in some embodiments at least about 75%, in some embodiments at least about 90%, or in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, w % ben present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

In some embodiments, the presently disclosed methods relate to treating and/or ameliorating symptoms that relate to diseases, disorders, and condition associated with undesirable demyelination. As used herein, the phrase “associated with undesirable demyelination” refers to any disease, disorder, and/or condition, or any symptom that is a consequence therefrom, that is results from undesirable demyelination, which in some embodiments is undesirable demyelination in the nervous system of a subject. Diseases, disorders, and condition associated with undesirable demyelination include, but are not limited to multiple sclerosis, spinal cord injuries, brain injuries, leukodystrophies, neuromyelitis optica (NMO), and Alzheimer's Disease (AD). In some embodiments, the disease, disorder, and/or condition is AD.

As used herein, the term “domain” refers to a part of a molecule or structure that shares common physicochemical features, such as, but not limited to, hydrophobic, polar, globular, and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be in some embodiments at least about 20 nucleotides in length, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350 nucleotides, in some embodiments at least about 350 nucleotides to about 500 nucleotides, in some embodiments at least about 500 to about 600, in some embodiments at least about 600 nucleotides to about 620 nucleotides, in some embodiments at least about 620 to about 650, and in some embodiments the nucleic acid fragment is greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules. e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters, expectation value 10.0, BLOSUM62 scoring matrix to obtain amino acid sequences homologous to a protein molecule described herein. To obtain gapped alignments for comparison purposes. Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material is may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

As used herein, the term “IL-9” refers to a gene that encodes an interleukin 9 protein and its transcription and translation products. The human IL-9 gene is located on chromosome 5 and corresponds to the reverse complement of nucleotides 135,892,246-135,895,841 of Accession No. NC_000005.10 of the GENBANK® biosequence database. Exemplary human IL-9 gene products include a transcription product disclosed as Accession No. NM_000590.2 (Homo sapiens interleukin 9 (IL9), mRNA; SEQ ID NO: 167) of the GENBANK® biosequence database, which encodes the IL-9 precursor protein of Accession No. NP_000581.1 of the GENBANK® biosequence database (SEQ ID NO: 168). Orthologs of IL-9 from non-human species include Accession Nos. NM_008373.2 and NP_032399.1 (mouse; SEQ ID NOs: 169 and 170, respectively), NM_001105747.1 and NP_001099217.1 (rat), NM_001037825.1 and NP_001032914.1 (chicken), XM_001168618.4 and XP_001168618.1 (chimpanzee), and XM_038681049.1 and XP_038536977.1 (dog) of the GENBANK® biosequence database, among others.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokarvote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”, sequences on the DNA strand which are located 5′ to a reference point on the DNA are referred to as “upstream sequences”: sequences on the DNA strand which are 3′ to a reference point on the DNA are referred to as “downstream sequences”.

Thus, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

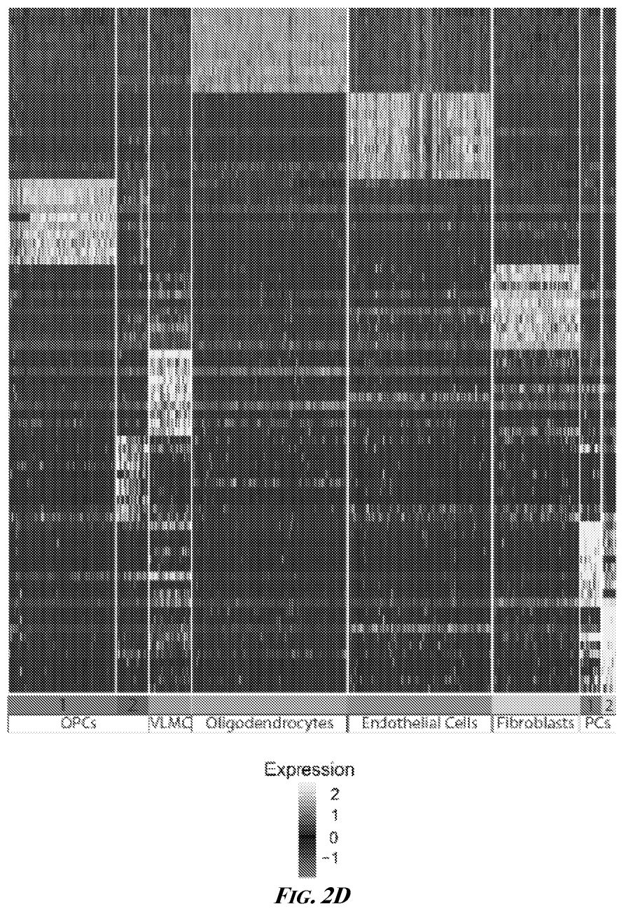

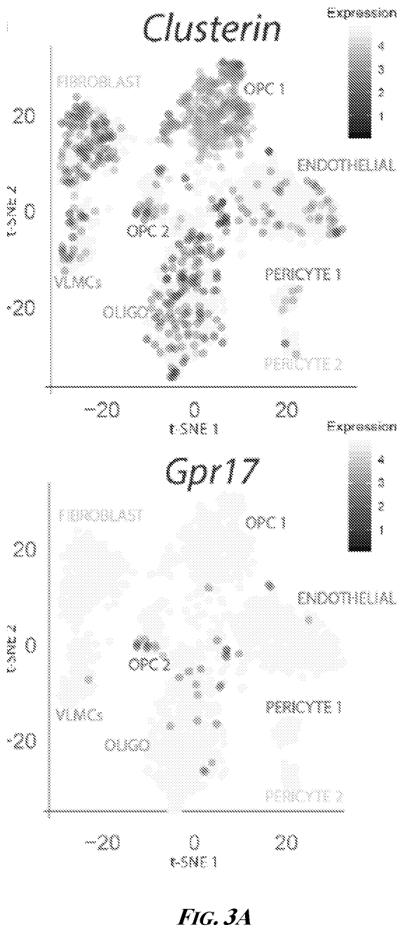

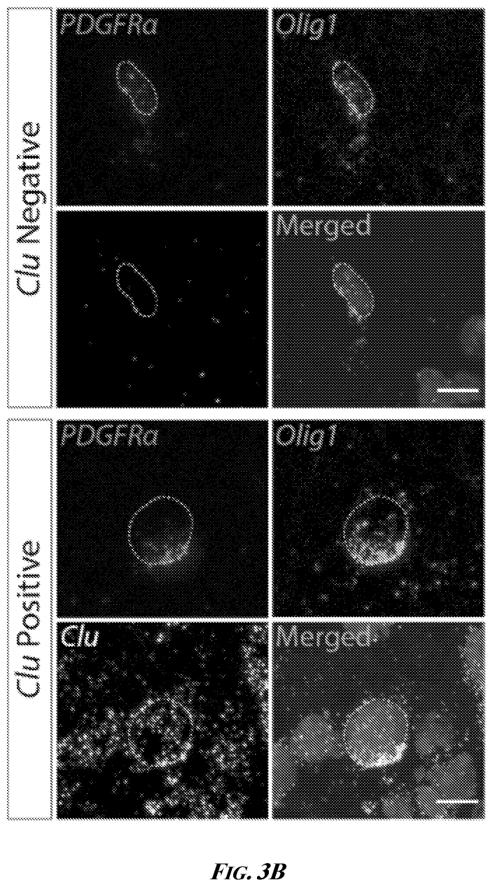

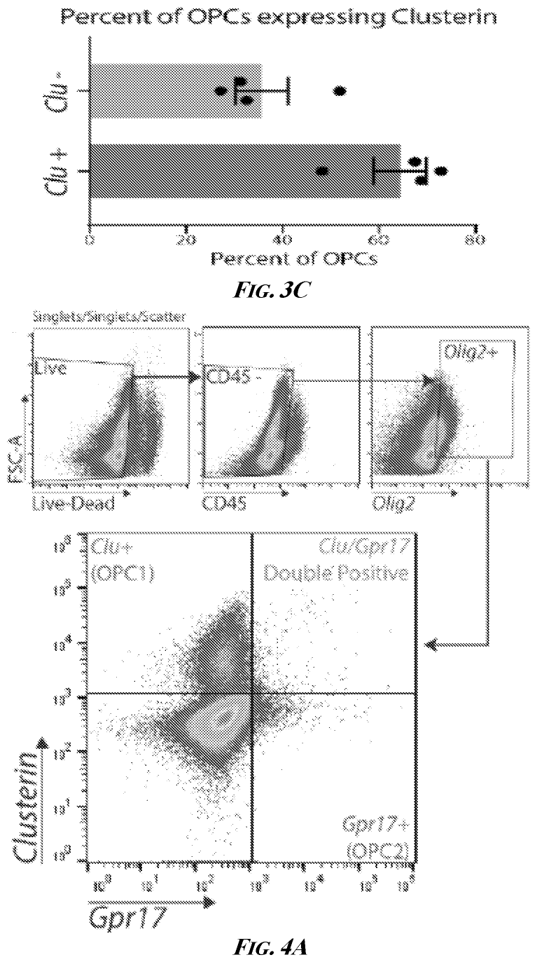

As used herein, the phrase “oligodendrocyte progenitor cell” (OPC) refers to a precursor cell that under appropriate conditions (e.g., under in vivo conditions) differentiates into a mature oligodendrocyte. Whereas various stem cells have the potential for differentiating into a mature oligodendrocyte, as used herein an OPC is a cell that can differentiate into a mature oligodendrocyte and expresses at least two of the following markers: oligodendrocyte transcription factor 1 (OLIG1 in humans; Accession Nos. NM_138983.3 and NP_620450.2 of the GENBANK® biosequence database), oligodendrocyte transcription factor 1 (OLIG2 in humans: Accession Nos. NM_005806.4 and NP_005797.1 of the GENBANK® biosequence database). Homo sapiens platelet derived growth factor receptor alpha (PDGFRA in human; Accession Nos. NM_006206.6 and NP_006197.1 of the GENBANK® biosequence database, among others), and chondroitin sulfate proteoglycan 4 (CSPG4OLIG2 in humans; Accession Nos. NM_001897.5 and NP_001888.2 of the GENBANK® biosequence database). See e.g., FIG. 3B.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein. “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By “small interfering RNAs (siRNAs)” is meant, inter alia, an isolated dsRNA molecule comprised of both a sense and an anti-sense strand. In some embodiments, it is greater than 10 nucleotides in length, siRNA also refers to a single transcript which has both the sense and complementary antisense sequences from the target gene, e.g., a hairpin, siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA).

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments humans.

As used herein, a “subject in need thereof” is a patient, animal, mammal, or human, who will benefit from the method of this presently disclosed subject matter. Thus, the term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and is methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are, in some embodiments 7% sodium dodecyl sulfate SDS, 0.5 M NaPO4, 1 mM EDTA at 50° C. with washing in 2× standard saline citrate (SSC), 0.1% SDS at 50° C.; in some embodiments 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), and viruses that incorporate the recombinant polynucleotide.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. EXEMPLARY EMBODIMENTS

### IIA. Compositions

In some embodiments, the presently disclosed subject matter relates to compositions that comprise inhibitors of clusterin biological activities. The presently disclosed subject matter encompasses the use of all types of inhibitors of the pathways described herein. The inhibitors include, but are not limited to, oligonucleotides and other nucleic acids such as but not limited to antisense oligonucleotides, siRNAs, shRNAs, and miRNA; antibodies and antibody fragments that bind to clusterin or downstream members of a clusterin biological pathway such as but not limited to anti-clusterin humanized antibodies, monoclonal antibodies, and fragments thereof; aptamer, phylomer, proteins that interact with clusterin and/or downstream members of a clusterin biological pathway to inhibit a biological activity of said clusterin or pathway, and small molecules such as but not limited to drugs.

Nucleic acids useful in the presently disclosed subject matter include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; miRNA, siRNA, and the like. The nucleic acids may be prepared by any conventional means typically used to prepare nucleic acids in large quantity. For example, DNAs and RNAs may be chemically synthesized using commercially available reagents and synthesizers by methods that are well-known in the art (see e.g., Gait, 1985). RNAs may be produce in high yield via in vitro transcription using plasmids such as SP65 (Promega Corporation. Madison, Wisconsin).

In some embodiments, an inhibitor of a clusterin biological activity is a nucleic acid-based inhibitor, optionally an siRNA or an miRNA that targets a clusterin gene product (including but not limited to a nucleotide sequence disclosed as NM_001831.4 of the GENBANK® biosequence database (SEQ ID NO: 7) or Accession No. NM_013492.3 of the GENBANK® biosequence database (SEQ ID NO: 9).

By “small interfering RNAs (siRNAs)” is meant, inter alia, an isolated dsRNA molecule comprised of both a sense and an anti-sense strand. In one aspect, it is greater than nucleotides in length, siRNA also refers to a single transcript which has both the sense and complementary antisense sequences from the target gene, e.g., a hairpin, siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides, siRNA technology has been described (see, for example, U.S. Pat. Nos. 6,506,559; 7,056,704; 8,372,968; and 8,420,391, the entire disclosure of each of which is incorporated herein by reference in their entirety).

The terms “microRNA” and “miRNA” are used interchangeably and refer to a nucleic acid molecule of about 17-24 nucleotides that is produced from a pri-miRNA, a pre-miRNA, or a functional equivalent, miRNAs are to be contrasted with short interfering RNAs (siRNAs), although in the context of exogenously supplied miRNAs and siRNAs, this distinction might be somewhat artificial. The distinction to keep in mind is that a miRNA is necessarily the product of nuclease activity on a hairpin molecule such as has been described herein, and an siRNA can be generated from a fully double-stranded RNA molecule or a hairpin molecule. Further information related to miRNAs generally, as well as a database of known published miRNAs and searching tools for mining the database can be found at the Wellcome Trust Sanger Institute miRBase: Sequences website, herein incorporated by reference. See also Griffiths-Jones, 2004, herein incorporated by reference. miRNA technology has been described (see, for example, U.S. Pat. Nos. 7,592,441; 7,825,229; 7,825,230; and 7,960,359, the entire disclosure of each of which is incorporated herein by reference in their entirety).

miRNAs are post-transcriptional regulators that bind to complementary sequences on target mRNAs. Although miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. Exemplary miRNAs that target CLU gene products include hsa-miR-15a-5p (SEQ ID NO: 88), mmu-miR-15a-5p (SEQ ID NO; 89), hsa-miR-15b-5p (SEQ ID NO: 90), hsa-miR-16-5p (SEQ ID NO: 91), hsa-miR-17-5p (SEQ ID NO: 92), hsa-miR-18a-3p (SEQ ID NO: 93), hsa-miR-21-5p (SEQ ID NO: 94), hsa-miR-25-5p (SEQ ID NO: 95), hsa-miR-29b-1 (SEQ ID NO: 96), hsa-miR-29b-2a (SEQ ID NO: 97), hsa-miR-29b-2b (SEQ ID NO: 98), hsa-miR-126a (SEQ ID NO: 99), hsa-miR-126b (SEQ ID NO: 100), hsa-miR-145a (SEQ ID NO: 101), hsa-miR-145b (SEQ ID NO: 102), hsa-miR-195-5p (SEQ ID NO: 103), mmu-miR-301b-3p (SEQ ID NO: 104), hsa-miR-326 (SEQ ID NO: 105), hsa-miR-330-5p (SEQ ID NO: 106), hsa-miR-424-5p (SEQ ID NO: 107), hsa-miR-425-5p (SEQ ID NO: 108), hsa-miR-485-5p (SEQ ID NO: 109), hsa-miR-497-5p (SEQ ID NO: 110), hsa-miR-518c-5p (SEQ ID NO: 111), hsa-miR-520a-5p (SEQ ID NO: 112), hsa-miR-525-5p (SEQ ID NO: 113), hsa-miR-571 (SEQ ID NO: 114), hsa-miR-650 (SEQ ID NO: 115), hsa-miR-873-5p (SEQ ID NO: 116), hsa-miR-89) (SEQ ID NO: 117), hsa-miR-922 (SEQ ID NO: 118), hsa-miR-1228-5p (SEQ ID NO: 119), hsa-miR-1237-5p (SEQ ID NO: 120), hsa-miR-1254 (SEQ ID NO: 121), hsa-miR-1275 (SEQ ID NO: 122), hsa-miR-1286 (SEQ ID NO: 123), hsa-miR-2467-3p (SEQ ID NO: 124), hsa-miR-2861 (SEQ ID NO: 125), hsa-miR-3116 (SEQ ID NO: 126), (SEQ ID NO:), hsa-miR-3152-5p (SEQ ID NO: 127), hsa-miR-3152-3p (SEQ ID NO: 128), hsa-miR-3175 (SEQ ID NO: 129), hsa-miR-3191-5p (SEQ ID NO: 130), hsa-miR-3612 (SEQ ID NO: 131), hsa-miR-3659 (SEQ ID NO: 132), hsa-miR-3678-3p (SEQ ID NO: 133), hsa-miR-4254 (SEQ ID NO: 134), hsa-miR-4259 (SEQ ID NO: 135), hsa-miR-4443 (SEQ ID NO: 136), hsa-miR-4486 (SEQ ID NO: 137), hsa-miR-4488 (SEQ ID NO: 138), hsa-miR-4505 (SEQ ID NO: 139), hsa-miR-4661-3p (SEQ ID NO: 140), hsa-miR-4665-5p (SEQ ID NO: 141), hsa-miR-4697-5p (SEQ ID NO: 142), hsa-miR-4706 (SEQ ID NO: 143), hsa-miR-4722-5p (SEQ ID NO: 144), hsa-miR-4731-5p (SEQ ID NO: 145), hsa-miR-4749-5p (SEQ ID NO: 146), hsa-miR-4781-5p (SEQ ID NO: 147), hsa-miR-5586-3p (SEQ ID NO: 148), hsa-miR-5787 (SEQ ID NO: 149), hsa-miR-6731-5p (SEQ ID NO: 150), hsa-miR-6751-5p (SEQ ID NO: 151), hsa-miR-6764-3p (SEQ ID NO: 152), hsa-miR-6777-5p (SEQ ID NO: 153), hsa-miR-6803-5p (SEQ ID NO: 154), hsa-miR-6817-3p (SEQ ID NO: 155), hsa-miR-6824-3p (SEQ ID NO: 156), hsa-miR-6838-5p (SEQ ID NO: 157), hsa-miR-6846-5p (SEQ ID NO: 158), hsa-miR-6848-5p (SEQ ID NO: 159), hsa-miR-6873-3p (SEQ ID NO: 160), hsa-miR-6884-5p (SEQ ID NO: 161), hsa-miR-6889-5p (SEQ ID NO: 162), hsa-miR-7109-5p (SEQ ID NO: 163), hsa-miR-7110-3p (SEQ ID NO: 164), hsa-miR-7855-5p (SEQ ID NO: 165), and hsa-miR-8085 (SEQ ID NO: 166). See also Table 2 below.

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, and recombinantly produced RNA. Thus, RNAs include, but are not limited to mRNA transcripts, miRNAs and miRNA precursors, and siRNAs. As used herein, the term “RNA” is also intended to encompass altered RNA, or analog RNA, which are RNAs that differ from naturally occurring RNA by the addition, deletion, substitution, and/or alteration of one or more nucleotides. Such alterations can include addition of non-nucleotide material, such as to the end(s) of the RNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In some embodiments, the double stranded region is at least 15 basepairs, in some embodiments between 15 and 300 basepairs, and in some embodiments between 15 and about 60 basepairs. As describe hereinabove, the formation of the double stranded region results from the hybridization of complementary RNA strands (for example, a sense strand and an antisense strand), either via an intermolecular hybridization (i.e., involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a short stretch of nucleotides (for example, about 5-10 nucleotides) such that the intramolecular hybridization event forms what is referred to in the art as a “hairpin” or a “stem-loop structure”.

By way of example and not limitation, an antisense nucleic acid that targets clusterin is OGX-011 (Lamoureux et al., 2011). OGX-011 has the nucleotide sequence 5-CAGCAGCAGAGTCTTCATCAT-3′ (SEQ ID NO: 11).

Thus, in some embodiments an inhibitor of a clusterin biological activity can be a small molecule can be an inhibitory nucleic acid. In some embodiments, the inhibitory nucleic acid targets a nucleic acid encoding a clusterin gene product. The use of inhibitory nucleic acids is exemplified in U.S. Patent Application Publication Nos. 2004/0009949, 2016/0024494, 2016/0362688, and 2020/0063102, each of which is incorporated by reference herein in its entirety.

In some embodiments, the inhibitory nucleic acid is an inhibitory RNA. In some embodiments, the inhibitory RNA targets a human clusterin gene product. As used herein the term “clusterin gene product” refers to a transcription product of a clusterin gene, optionally a human clusterin gene, or a translation product thereof. Commercially available anti-CLU inhibitory nucleic acids include those sold by OriGene Technologies, Inc. (Rockville, Maryland).

Alternatively or in addition, the CRISPR/Cas system can be employed to target a clusterin gene product. The use of CRISPR/Cas to alter gene expression is described in U.S. Pat. No. 8,697,359 and U.S. Patent Application Publication Nos. 2014/0189896, 2014/02426(4, 2014/0287838, and 2014/0357530, each of which is incorporated by reference in its entirety. Commercially available kits to target clusterin by CRISPR/Cas include those sold by GENSCRIPT® (Piscataway, New Jersey. United States of America; see also Sanjana et al., 2014), Applied Biological Materials Inc. (Richmon, British Columbia. Canada), Origene Technologies, Inc. (Rockville, Maryland, United States of America), VectorBuilder Inc. (Chicago, Illinois, United States of America), and Santa Cruz Biotechnology (Catalog No. sc-405452, Dallas, Texas, United States of America). Commercially available kits to target NR2 by CRISPR/Cas include those sold by GENSCRIPT® (Piscataway, New Jersey, United States of America; Catalog No. KN423623, which targets NR2B), and by Santa Cruz Biotechnology (Dallas, Texas, United States of America; e.g., Catalog No. sc-417830).

In some embodiments, the expression vector is designed to express a nucleic acid or polypeptide of the presently disclosed subject matter in a human cell after introduction of the vector into the cell or into a location where the expression vector can accumulate in the cell. In some embodiments, the nucleic acid expressed by the vector is an inhibitor nucleic acid that targets a clusterin gene product. In some embodiments, the polypeptide is an IL-9 polypeptide.

In some embodiments, the vector is a viral vector. In some embodiments, the virus is selected from adeno-associated virus (AAV), helper-dependent adenovirus, retrovirus, herpes simplex virus, lentivirus, poxvirus, hemagglutinatin virus of Japan-liposome (HVJ) complex, Moloney murine leukemia virus, and HIV-based virus. In some embodiments, the AAV capsid or inverted terminal repeats (ITRs) is selected from the group consisting of: AAV1, AAV2, AAV3, AAV4, AAV5, AAV6, AAV7, AAV8, AAV9, AAV10, AAV11. AAV12, rhl0, and hybrids thereof.

In some embodiments the vector is an AAV vector. AAV vectors are well known for use in expressing recombinant nucleic acids in cells including human cells. For example U.S. Patent Application Publication Nos. 2019/0000991 and 2019/0008909 (each of which is incorporated by reference in its entirety) discloses compositions and methods for AAV-based gene therapy in humans. See also U.S. Pat. Nos. 8,809,058; 9,540,659; 9,701,984; 9,840,719; 10,214,572; 10,392,632; and U.S. Patent Application Publication Nos. 2008/0206812; 2017/0157267; 2018/0311290; 2019/0002916; 2019/0048362; 2019/0060489.

Limitations of AAV vectors include inefficient production methods, packaging size constraints (introduced gene no larger than 4.5 kb), and a high level of immunity to AAV among adults (although AAV infection is not associated with any disease). The first AAV vectors were produced by transfection of 293 cells with two plasmids (an AAV vector plasmid and an AAV helper plasmid), and infection with adenovirus (reviewed in Muzyczka. 1992). This method provided the essential elements needed for AAV vector production, including AAV terminal repeat (TR) sequences flanking a gene of interest, AAV helper functions consisting of the rep and cap genes, and adenovirus genes.

Improvements to the basic method have included: delivery of adenovirus genes by transfection to eliminate contaminating adenovirus (Grimm et al., 1998; Matsushita et al., 1998; Xiao et al., 1998); delivery of AAV vector sequences within an Ad/AAV hybrid vector to increase vector production (Gao et al., 1998; Liu et al., 1999); and construction of first generation packaging cell lines containing the AAV rep and cap genes (Yang et al., 1994; Clark et al., 1995; Tamayose et al., 1996; Gao et al., 1998; Inoue & Russell, 1998; Liu et al., 1999).

In some embodiments, the viral vector of the presently disclosed subject matter can be measured as pfu (plaque forming units). In some embodiments, the pfu of recombinant virus, or viral vector of the compositions and methods of the presently disclosed subject matter can be about 10⁸ to about 5×10¹⁰ pfu. In some embodiments, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, recombinant viruses of this disclosure are at most about 1×10⁸, 2×10⁸, 3×10⁸, 4×10⁸, 5×10⁸, 5×10⁸, 6×10⁸, 7×10⁸, 8×10⁸, 9×10⁸, 1×10⁹, 2×10⁹, 3×10⁹, 4×10⁹, 5×10⁹, 6×,10⁹, 7×10⁹, 8×10⁹, 9×10⁹, 1×10¹⁰, 2×10¹⁰, 3×10¹⁰, 4×10¹⁰, and 5×10¹⁰ pfu.

In some embodiments, the viral vector of the presently disclosed subject matter can be measured as vector genomes. In some embodiments, recombinant viruses of this disclosure are 1×10¹⁰ to 3×10¹² vector genomes. In some embodiments, recombinant viruses of this disclosure are 1×10⁹ to 3×10¹³ vector genomes. In some embodiments, recombinant viruses of this disclosure are 1×10⁸ to 3×10¹⁴ vector genomes. In some embodiments, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes. In some embodiments, recombinant viruses of this disclosure are 1×10⁸ to 3×10¹⁴ vector genomes. In some embodiments, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some embodiments, the viral vector of the presently disclosed subject matter can be measured using multiplicity of infection (MOI). In some embodiments, MOI may refer to the ratio, or multiple of vector or viral genomes to the cells to which the nucleic may be delivered. In some embodiments, the MOI may be 1×10⁶. In some embodiments, the MOI may be 1×10⁵-1×10⁷. In some embodiments, the MOI may be 1×10⁴-1×10⁸. In some embodiments, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some embodiments, recombinant viruses of this disclosure are 1×10⁸ to 3×10¹⁴ MOI. In some embodiments, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some embodiments the nucleic acid may be delivered without the use of a virus (i.e. with a non-viral vector), and may be measured as the quantity of nucleic acid. Generally, any suitable amount of nucleic acid may be used with the compositions and methods of this disclosure. In some embodiments, nucleic acid may be at least about 1 pg, 10 pg, 100 pg, 1 pg, 10 pg, 10 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 70) mg, 800 mg, 900 mg 1 g, 2 g, 3 g, 4 g, or 5 g. In some embodiments, nucleic acid may be at most about 1 pg, 10 pg, 100 pg, 1 pg, 10 pg, 100 pg, 200 pg, 300 pg, 400 pg, 500 pg. 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some embodiments, a self-complementary vector (sc) may be used. The use of self-complementary AAV vectors may bypass the requirement for viral second-strand DNA synthesis and may lead to greater rate of expression of the transgene protein, as provided by Wu, 2007, incorporated by reference herein.

In some embodiments, several AAV vectors may be generated to enable selection of the most optimal serotype, promoter, and transgene.

In some embodiments, the vector can be a targeted vector, especially a targeted vector that selectively binds to a specific cell, such as cancer cells or tumor cells or eye cells. Viral vectors for use in the disclosure can include those that exhibit low toxicity to a target cell and induce production of therapeutically useful quantities of the anti-VEGF protein in a cell specific manner.

The compositions and methods of the disclosure provide for any suitable viral nucleic acid delivery systems including but not limited to use of at least one of an adeno-associated virus (AAV), adenovirus, helper-dependent adenovirus, retrovirus, herpes simplex virus, lentivirus, poxvirus, hemagglutinatin virus of Japan-liposome (HVJ) complex, Moloney murine leukemia virus, and HIV-based virus. In some embodiments, the viral vector comprises a strong eukaryotic promoter operably linked to the polynucleotide e.g., a cytomegalovirus (CMV) promoter.

Generally, any suitable viral vectors may be engineered to be optimized for use with the compositions and methods of the disclosure. For example, viral vectors derived from adenovirus (Ad) or adeno-associated virus (AAV) may be used. Both human and non-human viral vectors can be used and the recombinant viral vector can be altered such that it may be replication-defective in humans. Where the vector is an adenovirus, the vector can comprise a polynucleotide having a promoter operably linked to a gene encoding the anti-VEGF protein and is replication-defective in humans.

To combine advantageous properties of two viral vector systems, hybrid viral vectors may be used to deliver a nucleic acid encoding a nucleic acid or polypeptide to a target cell or tissue. Standard techniques for the construction of hybrid vectors are well-known to those skilled in the art. Such techniques can be found, for example, in Green & Sambrook et al., 2012 or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., 1999. Retrovirus/adenovirus hybrid vectors are discussed in Zheng et al., 2000.

Retroviral genomes contained within an adenovirus may integrate within the target cell genome and effect stable gene expression.

Replication-defective recombinant adenoviral vectors can be produced in accordance with known techniques. See e.g., Quantin et al., 1992: Stratford-Perricadet et al., 1992; Rosenfeld et al., 1992.

Additional exemplary vectors may include but are not limited to viral vectors, fusion proteins and chemical conjugates. Retroviral vectors include Moloney murine leukemia viruses and HIV-based viruses. In some embodiments a HIV-based viral vector may be used, wherein the HIV-based viral vector comprises at least two vectors wherein the gag and pol genes are from an HIV genome and the env gene is from another virus. DNA viral vectors may be used. These vectors include pox vectors such as orthopox or avipox vectors, herpesvirus vectors such as a herpes simplex I virus (HSV) vector (see e.g., Geller et al., 1990: Geller et al., 1993; Geller et al., 1995; Lim et al., 1995), Adenovirus Vectors (see e.g., LeGal LaSalle et al., 1993; Davidson et al., 1993; Yang et al., 1995), and Adeno-associated Virus Vectors (see e.g., Kaplitt et al., 1994), each of which is incorporated by reference herein.

In some embodiments, an inhibitor of a clusterin biological activity can be an anti-clusterin antibody. Anti-clusterin antibodies include those described in U.S. Pat. No. 9,512,211, which is incorporated herein by reference in its entirety. Commercially available anti-clusterin antibodies include those sold by Abcam plc. (Waltham, Massachusetts, United States of America), Enzo Life Sciences, Inc. (Farmingdale, New York, United States of America), and Santa Cruz Biotechnology, Inc. (Dallas, Texas. United States of America).

### II.A.1. Pharmaceutical Compositions

In some embodiments, the compositions of the presently disclosed subject matter are provided as part of a pharmaceutical composition. As used herein, the term “pharmaceutical composition” refers to a composition comprising at least one active ingredient (e.g., an inhibitor of the presently disclosed subject matter), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of at least one active ingredient (e.g., an inhibitor of the presently disclosed subject matter) and a pharmaceutically acceptable diluent and/or excipient. As used herein, the term “pharmaceutically acceptable” refers to physiologically tolerable, for either human or veterinary application. Similarly. “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

In some embodiments, the pharmaceutical compositions of the presently disclosed subject matter are for use in preventing and/or treating a disease or disorder associated with genotoxic stress-induced cardiac toxicity in a subject in need thereof.

The pharmaceutical compositions of the presently disclosed subject matter can in some embodiments consist of the active ingredient alone, in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

### II.A.2. Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the invention suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methy lcellulose, hydroxypropylmethylcellulose.

Known dispersing or wetting agents include, but are not limited to, naturally occurring phosphatides such as lecithin, condensation products of an alkvlene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for parenteral administration, including but not limited to intraocular injection.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents: fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, 1985, which is incorporated herein by reference in its entirety.

### II.A.3. Administration

With regard to administering a composition of the presently disclosed subject matter, methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

### II.A.4. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### II.B. Methods

The presently disclosed subject matter also relates in some embodiments to methods for treating a disease, disorder, or condition associated with undesirable demyelination and/or ameliorating at least one symptom thereof, optionally wherein the a disease, disorder, or condition associated with undesirable demyelination is Alzheimer's Disease (AD), in subjects in need thereof. In some embodiments, the methods comprise administering to a subject with a disease, disorder, or condition associated with undesirable demyelination, optionally AD, a composition comprising an inhibitor of a clusterin biological activity, wherein the composition is administered via a route and in an amount sufficient to inhibit the clusterin biological activity to thereby treat the subject's a disease, disorder, or condition associated with undesirable demyelination and/or ameliorate at least one symptom thereof. In some embodiments, the administering reduces an amount of clusterin in at least one cell type of the central nervous system (CNS) of the subject, optionally in the brain of the subject.

As used herein, the phrase “clusterin biological activity” refers to any compound or composition that reduces any clusterin biological activity in a cell, tissue, or organ in vitro or in vivo. Exemplary biological activities that have been attributed to clusterin include preventing apoptosis, inhibiting the complement cascade, and promoting clearance of cellular debris (Murphy et al., 1988; Falgarone & Chiocchia. 2009; Wyatt et al., 2011; Pereira et al., 2018).

The presently disclosed subject matter also relates in some embodiments to methods for reducing and/or inhibiting myelin decay in a subject in need thereof, the method comprising administering to the subject a composition comprising an inhibitor of a clusterin biological activity, wherein the composition is administered via a route and in an amount sufficient to reduce and/or inhibit myelin decay in the subject. In some embodiments, the subject has or is at risk for developing a disease, disorder, or condition associated with undesirable demyelination, which in some embodiments can be multiple sclerosis; spinal cord injury, brain injury, leukodystrophies, neuromyelitis optica (NMO), and/or Alzheimer's Disease (AD). Those who are at risk for developing diseases, disorders, and/or condition associated with undesirable demyelination such as Alzheimer's include those with family histories of such diseases (e.g., AD), including but not limited to those with parents and/or siblings that have AD. By way of example and not limitation, there have been certain genetic markers that are associated with early-onset AD, including amyloid precursor protein (APP; human chromosome 21) presenilin 1 (PSEN1; human chromosome 14), and presenilin 2 (PSEN2: human chromosome 1. It is noted that in the context of the presently disclosed and claimed subject matter, the phrase “at risk for developing Alzheimer's Disease” also encompasses subjects who are at risk for developing a worsening of already detectable AD symptoms.

As used herein, the phrase “myelin decay”, also referred to as “myelin degeneration”, refers to any disease, disorder, or condition that causes or results in damage to the myelin sheath of nerve fibers in the nervous system, including but not limited to the brain. Myelin decay can also occur in the optic nerves and in the spinal cord. A subject who experiences myelin decay and/or myelin degeneration is one referred to here as a subject who has a disease, disorder, or condition associated with undesirable demyelination.

In some embodiments of the presently disclosed methods, the inhibitor of a clusterin biological activity comprises an inhibitory nucleic acid that binds to and reduces translation of a clusterin gene product, optionally a human clusterin gene product. In some embodiments, the inhibitory nucleic acid targets a subsequence of a human clusterin gene product as set forth in Accession No. NM_001831.4 of the GENBANK® biosequence database. In some embodiments, the inhibitor of a clusterin biological activity comprises a guide RNA (gRNA) that comprises a sequence that comprises, consists essentially of, or consists of a nucleotide sequence selected from the group consisting of CGTCTATGATGCTGGACGCG (SEQ ID NO: 2), GACGTACTTACTTCCCTGAT (SEQ ID NO: 3), and GCGTGCGTAGAACTTCATGC (SEQ ID NO: 6) and/or that targets a clusterin gene product nucleotide sequence that comprises, consists essentially of, or consists of a nucleotide sequence selected from the group consisting of TACGCACGCGTCTGCAGAAG (SEQ ID NO: 1), AGAAGGCGACGATGAC (SEQ ID NO: 4), and CCGCCAACAGAATTCATACG (SEQ ID NO: 5).

### II.C. Combination Therapies

In some embodiments, the methods of the presently disclosed subject matter are combined with other treatment and prevention methodologies. In some embodiments, the presently disclosed methods further comprise administering to the subject an additional treatment, optionally an additional AD treatment. In some embodiments, the additional treatment is selected from the group consisting of treatment with an acetylcholinesterase (AChE) inhibitor, optionally donepezil (2-[(1-benzylpiperidin-4-yl)methyl]-5,6-dimethoxy-2,3-dihydro-1H-inden-1-one; U.S. Pat. Nos. 4,895,841; 7,727,548; 7,727,552; 8,039,009; 8,173,708; 8,283,379; 8,329,752; 8,362,085), rivastigmine (3-[(1S)-1-(dimethylamino)ethyl]phenyl N-ethyl-N-methylcarbamate; U.S. Pat. Nos. 4,948,807; 6,316,023; 6,335,031), and/or galantamine ((1S,12S,14R)-9-methoxy-4-methyl-11-oxa-4-azatetracyclo[8.6.1.0^{1,12}.0^{6,7}]heptadeca-6(17),7,9,15-tetraen-14-ol; U.S. Pat. Nos. 6,099,863; 6,358,527; 7,160,559); treatment with an N-methyl-d-aspartate receptor (NMDAR) antagonist, optionally, memantine (3,5-dimethyladamantan-1-amine; U.S. Pat. Nos. 8,039,009; 8,058,291; 8,168,209; 8,173,708; 8,283,379; 8,329,752; 8,338,486; 8,362,085; 8,598,233); treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126 (e.g., SEQ ID NO: 99 or 100), miR-145 (e.g., SEQ ID NO: 101 or 102), miR-195 (e.g., SEQ ID NO: 103), miR-21 (e.g., SEQ ID NO: 94), and miR-29b (e.g., SEQ ID NO: 96, 97, or 98; see e.g., Wang et al., 2019); treatment with a reverse transcriptase inhibitor (see PCT International Patent Application Publication No. WO 2020/185676; and combinations thereof. See also U.S. Pat. No. 9,365,647 and U.S. Patent Application Publication Nos. 2018/0177790 and 2020/0071699, which along with the references cited hereinabove are incorporated by reference in their entireties.

Exemplary secretase inhibitors include both β- and γ-secretase inhibitors, including but not limited to anti-secretase antibodies and paratope-containing fragments thereof, anti-secretase nucleic acids targeted to secretase gene products similar to those described herein for clusterin (e.g., nucleic acids that are designed to bind to and thus inhibit a biological activity of a secretase gene product), and small molecule inhibitors.

Similarly, exemplary BACE inhibitors include anti-BACE antibodies and paratope-containing fragments thereof, anti-BACE nucleic acids targeted to BACE gene products similar to those described herein for clusterin (e.g., nucleic acids that are designed to bind to and thus inhibit a biological activity of a BACE gene product), and small molecule inhibitors. By way of example and not limitation, fused aminodihydrothiazine derivatives that possess BACE inhibitory activity include those disclosed in U.S. Patent Application Publication No. 2009/0209755, (S)-4-(2,4-difluoro-5-pyrimidin-5-yl-phenyl)-4-methyl-5,6-dihydro-4H-[1,3]thiazin-2-ylamine, an orally administered CNS-active BACE inhibitor (see May et al., 2011).

Exemplary inhibitors of tau aggregation include anti-tau antibodies and paratope-containing fragments thereof, anti-tau nucleic acids targeted to tau gene products similar to those described herein for clusterin (e.g., nucleic acids that are designed to bind to and thus inhibit a biological activity of a tau gene product), and small molecule inhibitors. See e.g., Wischik et al., 1996; Necula et al. 2005; Pickhardt et al., 2005; Taniguchi et al., 2005a; Taniguchi et al., 2005b; Larbig et al., 2007.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Introduction to Examples 1-5

While performing single cell RNAseq on adult OPCs, it was determined that a subset of OPCs expressed clusterin. It was further determined that oligomeric Aβ and myelin debris were able to drive a pathological increase in clusterin expression in OPCs. More importantly, it was determined that treating OPCs with clusterin blocked their differentiation into mature oligodendrocytes. Based on this strong scientific premise, it was hypothesized that clusterin acts an inhibitor of myelin repair by diverting OPCs away from differentiation into myelinating oligodendrocytes.

### Materials and Methods for the Examples

Statistical analysis, scientific rigor and consideration of relevant biological variables: All of the transgenic mouse strains and littermate controls used in the disclosed studies are bred in house. We understand and appreciate the importance of sex as a biological variable critical to the interpretation, validation, and generalizability of our research findings. All major phenotypes are validated in both sexes in our studies and all experiments will be conducted with sex and age-matched controls. All mice are on an inbred genetic background (C57BL/6) and we always compare wildtype (WT) and transgenic mice on the same genetic background and confirm all phenotypes using littermate controls. To enhance rigor, all experiments and analyses are performed in a blinded fashion. To ensure reproducibility, all findings are repeated at least once (two independent experiments) and major ones at least twice (three independent experiments).

Statistical analysis: Based on power analysis and previous studies from colleagues, we it has been have determined that the evaluation of spatial learning and memory (Morris water maze test) requires 10 mice per group to achieve a power score of 0.8. Based on preliminary results, it has also been determined that 10 mice per group are required to achieve a power score of 0.8 for studies in Aim 1. Statistical significance (P<0.05) is determined using a Student's t-test (two groups) or a one-way analysis of variance (ANOVA) (more than two groups) with Tukey's post hoc test. Comparisons between groups over time, or other data sets with nonparametric data will be made by two-way ANOVA with Bonferroni's post hoc test.

### Example 1

### Evidence for OPC Diversity in the Adult Mouse Brain

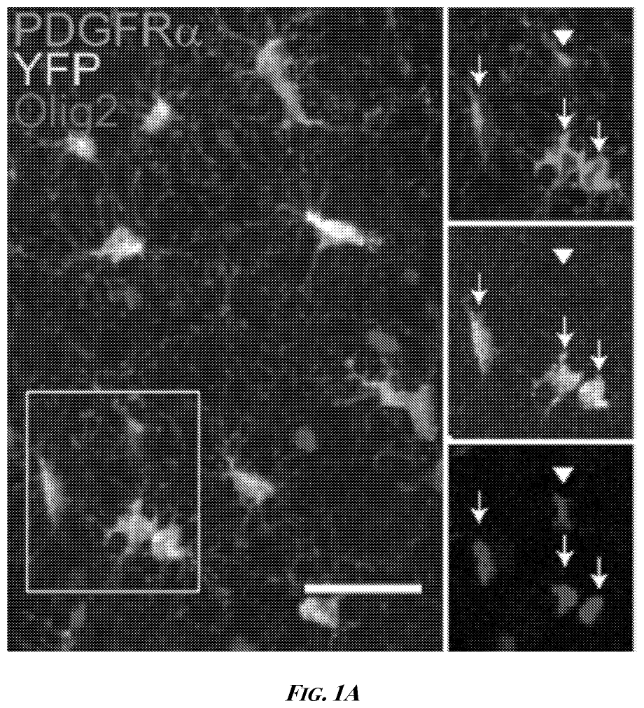

In order to explore OPC diversity in the adult brain, we generated reporter mice by crossing the PDGFRα Cre-ERT2 strain (Jackson Laboratories, Catalog No. 018280) to ROSA26 EYFP animals (Jackson Laboratories, Catalog No. 006148; see also Baxi et al., 2014). Since a goal was to study OPC diversity in adult mice, an inducible CRE was selected to avoid labelling oligodendrocytes generated during developmental myelination. We began by confirming that YFP expression can be induced in OPCs following tamoxifen administration; ten days post treatment we observed robust YFP expression in OPCs (OLIG2+/PDGFRα+ cells; see FIG. 1A).

To investigate the optimal dose of tamoxifen required for the reporter expression, we treated adult mice (6 weeks old) with 1, 2, or 3 tamoxifen injections (each injection was administered 3 days apart) and counted the number of YFP positive OPCs (OLIG2+/PDGFRα+) ten days after the first injection (see FIG. 1B). We discovered that 2 tamoxifen injections were optimal to label 95% of the OPCs (see FIG. 1B).

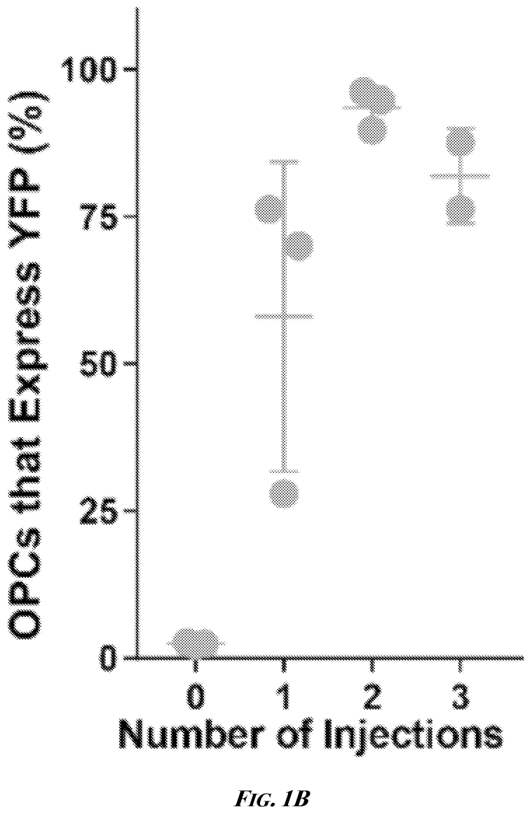

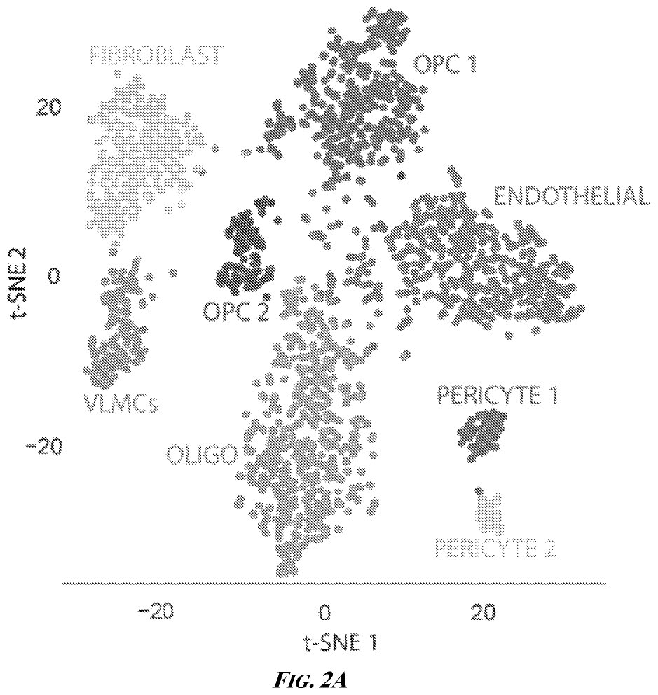

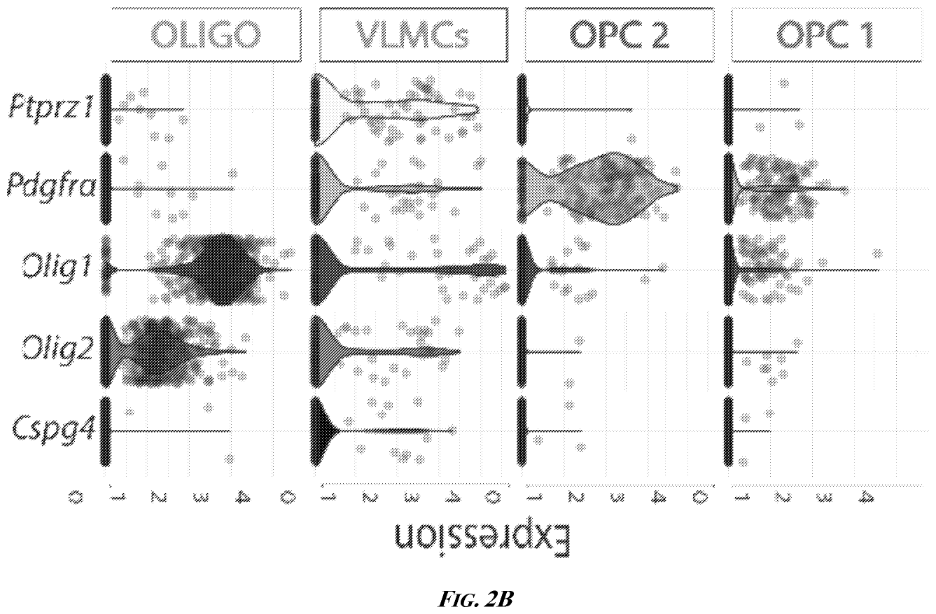

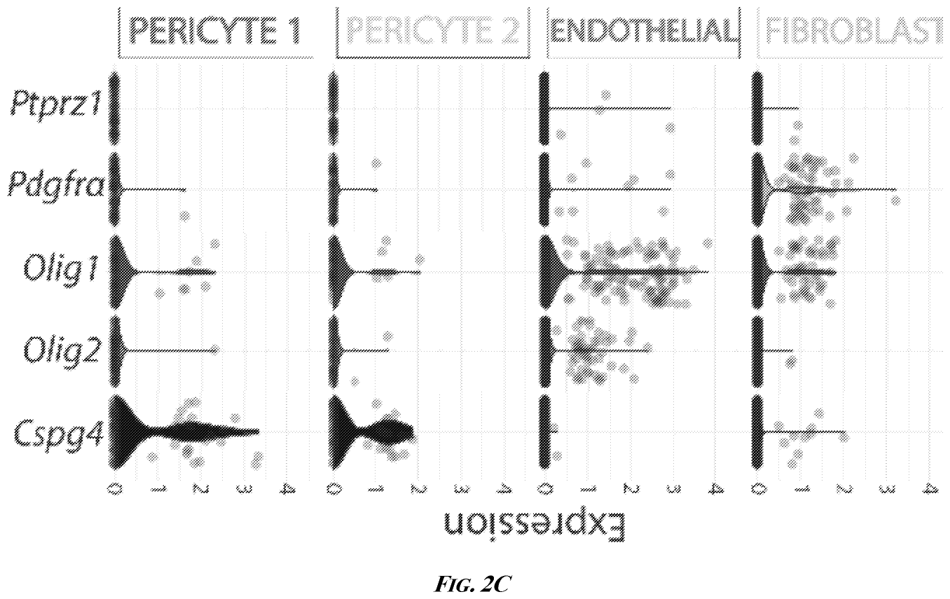

We next sorted YFP+ cells from adult mice using our OPC reporter strain and performed single cell sequencing (N=3). Unbiased clustering of sequenced cells using the Seurat package (Butler et al., 2018; Stuart et al., 2019) revealed that cells sorted from PDGFRα-CreER; R26-EYFP brains clustered into 8 distinct populations (see FIG. 2A). Mature oligodendrocytes comprised one cluster, having potentially differentiated following initial tamoxifen labeling of PDGFRα-expressing progenitor cells. Also captured in the sequencing were four cell types outside the oligolineage that are known to either express PDGFRα or come from PDGFRα expressing precursors, including one population of fibroblasts, one population of endothelial cells, one population of vascular and leptomeningeal cells (VLMCs), and two populations of pericytes (Marques et al., 2016; Aghajanian et al., 2017; Endale et al., 2017; Li et al., 2018; Marques et al., 2018). These clusters were identified by expression of known cell type markers such as Igfbp6 and Fn1 (fibroblasts), Tek, Pecam1, and Kdr (endothelial cells), Lum, Col1a2, and Col3a1 (VLMCs), as well as Rgs5, Pdgfrb, and Des (pericytes; see also Bondjers et al., 2003; Witmer et al., 2004; Marques et al., 2016; Xiao et al., 2017; Smyth et al., 2018). The remaining 2 clusters of cells, which we have called OPC1 and OPC2 expressed at least 2 canonical OPC markers (Ptprz, PDGFRα, Olig1, Olig2, and Cspg4; see FIGS. 2B and 2C) and expressed a unique transcriptional signature distinct from the gene expression in every other cluster (see FIG. 2D; see also Hall et al., 1996; Nishiyama et al., 1996; Lu et al., 2000; Yao et al., 2014; Falcao et al., 2018).

### Example 2

### A Subset of Adult OPCs Express Clusterin In Vivo

To discover putative functions associated with these two clusters of OPCs, we began by identify transcripts that were highly specific to each cluster. OPC1 expressed high levels of clusterin, a multifunctional protein that has been identified as a risk factor for late-onset Alzheimer Disease (LOAD; Gu et al., 2011). OPC2 was positive for G-protein coupled receptor Gpr17, the only currently known marker of molecular diversity in OPCs (see FIG. 3A; see also Fumagalli et al., 2011; Vigano et al., 2016). Since gene expression can be altered by tissue processing before sequencing, we validated the expression of clusterin in OPCs using RNAscope in the adult mouse brain (van den Brink et al., 2017). OPCs were defined by the co-expression of two canonical OPC transcripts consisting of Pdgfra, a cell-surface receptor, and the oligolineage transcription factor Olig1. Using a computational approach to unbiasedly quantify RNA puncta expression per cell (Erben et al., 2018), we were able to identify clusterin negative (40%, ≤10 puncta/nuclei) and clusterin positive OPC (60%, >10 puncta/nuclei) in the adult brain (see FIGS. 3B and 3C).

Next, we explored if Clu and Gpr17 can be co-expressed by adult OPCs or if they are unique markers of distinct OPC clusters. Using the PRIMEFLOW™ brand RNA assay (ThermoFisher Scientific, Waltham. Massachusetts, United States of America), a technique that allows for the combination of cellular-resolution RNA detection with the multiplexing capabilities of flow cytometry, we demonstrated that a subset of Olig2 expressing cells express clusterin, and a mutually exclusive population expresses Gpr17, with very few cells co-expressing detectable levels of both markers (see FIGS. 4A and 4B). While this does not rule out the possibility that an individual OPC might express genes enriched in different clusters at different times, it does demonstrate that, at any given point, genetic markers of these two clusters of OPCs largely do not overlap. In sum, our preliminary data demonstrated that a subset of OPCs express clusterin in vivo.

### Example 3

### Clusterin Expression is Elevated in AD Patients and an AD Animal Model

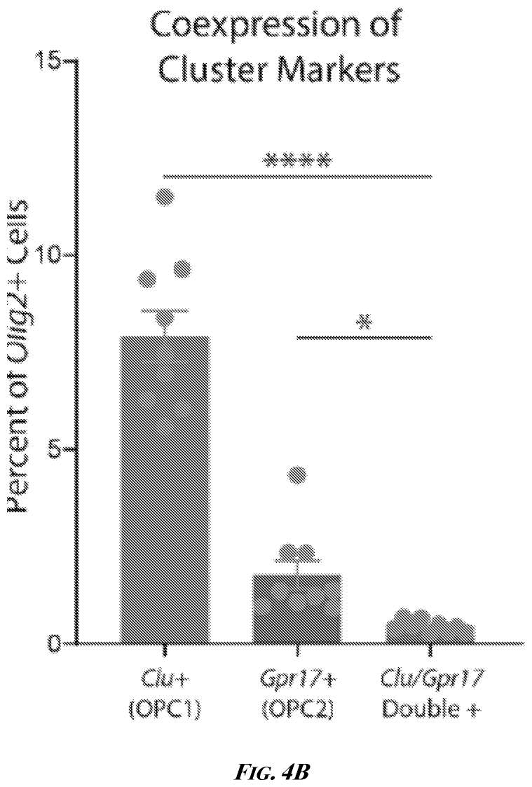

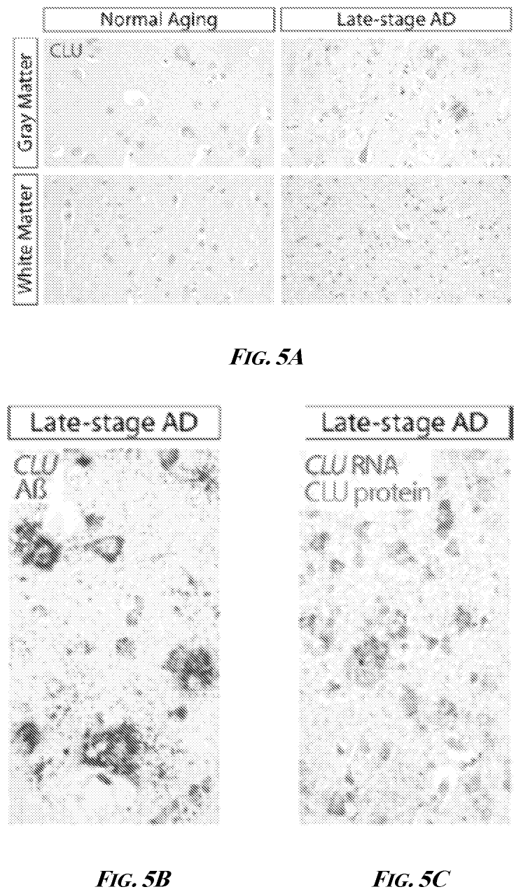

A very common SNP in the CLU gene (rs11136000; Lambert et al., 2009) has been documented to be a significant risk factor for LOAD (DeMattos et al., 2001; Bertram et al., 2007; Bertram & Tanzi, 2009; Harold et al., 2009). Furthermore, clusterin levels are elevated in AD patients as well as animal models of AD (Hong et al., 2013; Miners et al., 2017). Due to the methodologies used in prior studies documenting this upregulation of clusterin (ELISA and mass spectrometry), the anatomical locations and the cell types involved in clusterin expression were not described. We performed clusterin IHC on AD and normal aging patients and noted stronger clusterin immunoreactivity in AD patients (see FIG. 5A). By combining IHC for Ab and ISH for clusterin, we were able to detect that cells surrounding the AD plaques express clusterin transcript (see FIG. 5B). Combination of IHC and ISH for clusterin allows to demonstrate how cells adjacent to plaques secrete clusterin (see FIG. 5C). This upregulation of clusterin was also detected in the cortex and hippocampus of the 5×FAD AD model using immunofluorescence (see FIGS. 5D and 5E). Our preliminary data suggested that clusterin is upregulated in the brains of AD patients as well as in the 5×FAD model, corroborating existing literature (Hong et al., 2013; Miners et al., 2017). The experiments proposed in this application will uncover the cell types responsible for overexpression of clusterin in AD patients and 5×FAD mice. Based on our sequencing data, we anticipate that OPCs will contribute to this pathological upregulation of clusterin.

### Example 4

Exogenous Clusterin Blocks OPC Differentiation into Oligodendrocytes

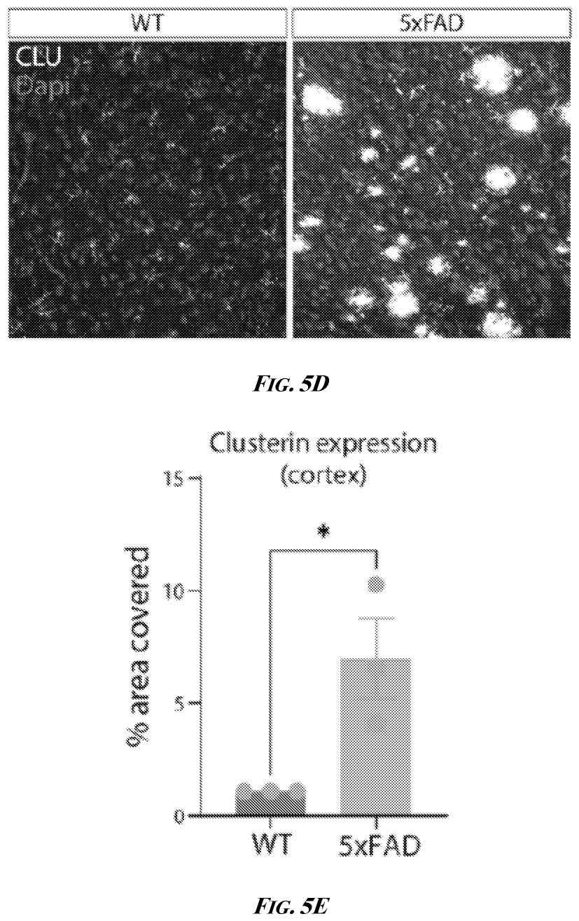

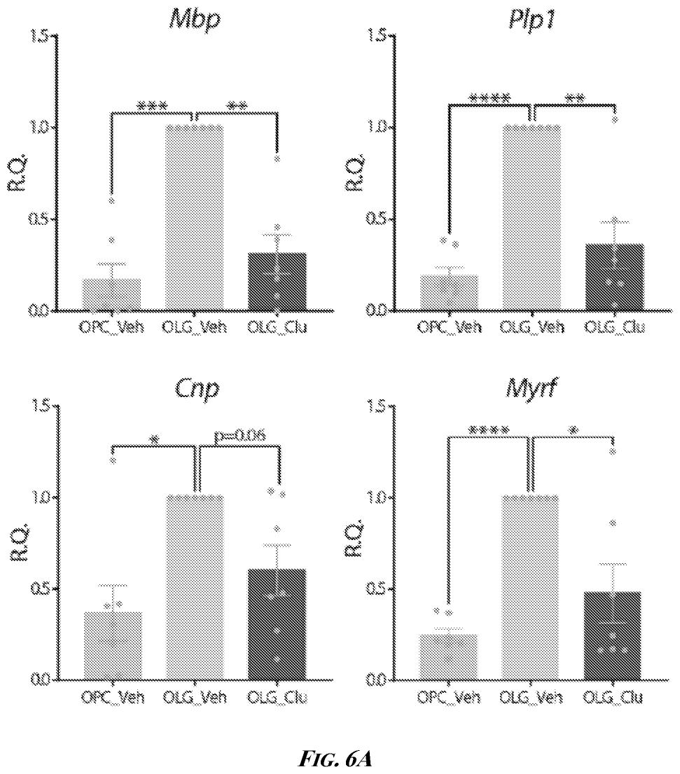

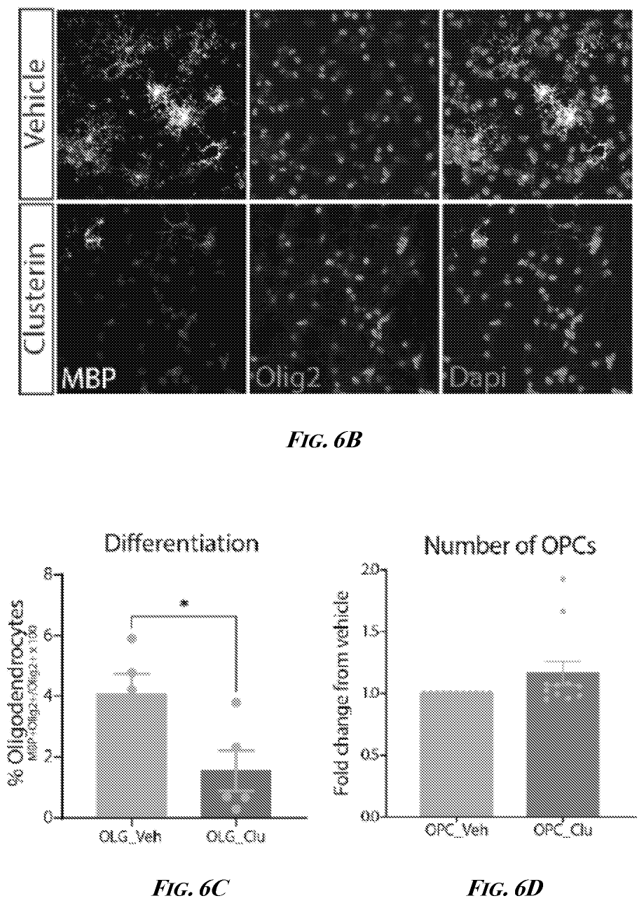

Given data demonstrating that clusterin expression is elevated in AD (Hong et al., 2013; Miners et al., 2017) and that myelin has been reported to be impacted in AD patients and AD preclinical models (Roher et al., 2002; Desai et al., 200); Desai et al., 2010; Zhan et al., 2014), we explored the impact of exogenous clusterin on the differentiation of OPCs into myelinating oligodendrocytes. OPCs were prepared from neurospheres as previously described (Franco et al., 2015) and subsequently switched into differentiating conditions in the presence of recombinant clusterin (8 μg/ml) or vehicle (PBS) for 3 days. Expression of myelin markers (Mbp, Plp, Cnp, and Myrf) as determined by qPCR was decreased in clusterin treated OPCs (see FIG. 6A). We also demonstrated a decrease in the percentage of MBP positive oligolineage cells using immunofluorescence (see FIGS. 6B and 6C). Importantly, clusterin did not impact OPC viability as determined by CCK8 assay (see FIG. 6D). Taken together, our data supported the hypothesis that pathological expression of clusterin could impact OPC differentiation and this might explain the myelin defect present in AD and AD preclinical models.

### Example 5

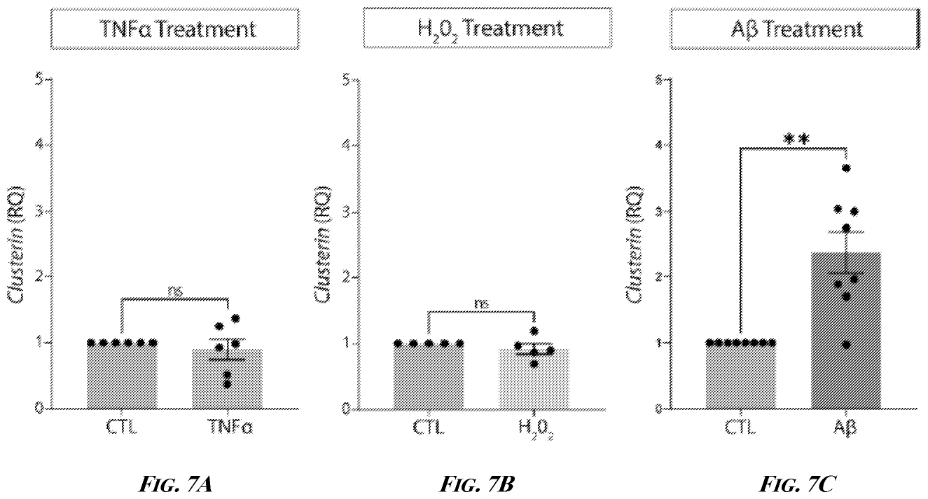

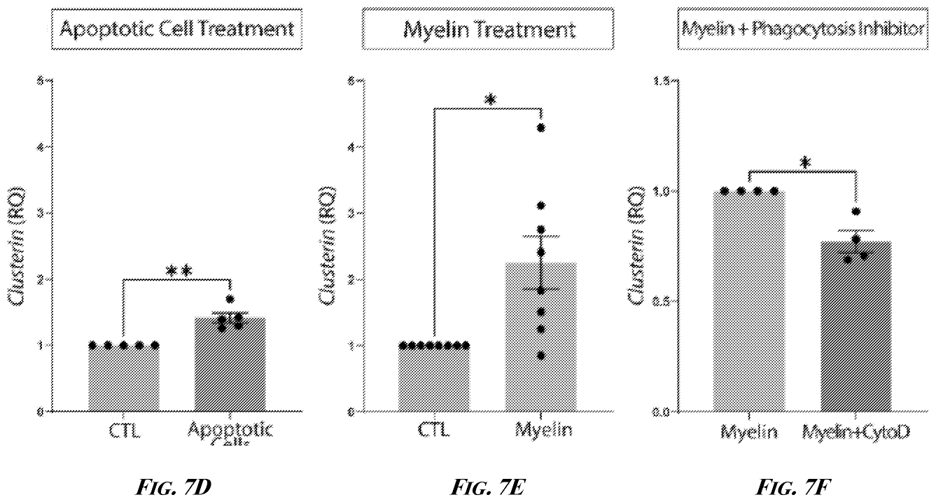

Oligomeric AD Drives Expression of Clusterin in OPCs Given the impact of clusterin on OPCs differentiation in vitro, and the evidence supporting that myelin is impacted in AD (Roher et al., 2002; Desai et al., 2009; Desai et al., 2010; Zhan et al., 2014), we wanted to understand what factors initiate expression of clusterin in OPCs. We treated primary OPCs with TNFa, H2O2, oligomeric Aβ, myelin debris, and apoptotic cells and monitored expression of clusterin by qPCR. These treatments were selected because of their association with AD pathology (Sengupta et al., 2016; Ahmad et al., 2017; Chang et al., 2017). While TNFa and H2O2 at the concentration tested did not influence clusterin expression in OPCs (see FIGS. 7A and 7B), oligomeric Aβ induced a significant upregulation of clusterin (see FIG. 7C). Clusterin expression was also increased following treatment with myelin debris and apoptotic cells (see FIGS. 7D and 7E). These results suggest that macromolecules and protein aggregates can promote clusterin expression perhaps pointing to a link with phagocytosis. In order to test if phagocytosis was required to mediate the upregulation of clusterin, the same experiment was repeated in the presence of Cytochalasin D to block actin polymerization and prevent engulfment (Axline & Reaven, 1974; Soldati & Schliwa, 2006). Our results suggest that internalization of the cargo is necessary to mediate the upregulation of clusterin (see FIG. 7F).

### Introduction to Examples 6-9

Preliminary data suggested that engulfment of large biological objects by OPCs is necessary for clusterin upregulation. Furthermore, we demonstrate that exogenous clusterin blocks OPC differentiation into oligodendrocytes, potentially explaining the reason why clusterin is a risk factor for AD. Here, we propose to decipher the agents leading to clusterin upregulation by OPCs using in vitro culture of OPCs. Proteomics is employed to discover the mechanism of action of clusterin on OPCs.

### Example 6

### OPC Differentiation and Myelination In Vitro

To investigate the effect of exogenous clusterin on OPC differentiation, primary OPCs are isolated as described in Fernandez-Castaneda et al., 2020. OPCs are switched to differentiation media to induce OPC maturation and are cultured with increasing concentrations is of clusterin or with a vehicle control (Fernandez-Castaneda et al., 2020). Maturation of OPCs into oligodendrocytes is followed by immunofluorescence analysis of OPC and oligodendrocyte markers after 3 days of culture. Markers are used to confirm that cells are OPCs (PDGFR-α and OLIG2) or oligodendrocytes (CC1, CNP, PLP and MBP; Baumann & Pham-Dinh, 2001). OPC apoptosis is monitored by staining for PDGFR-α and activated caspase 3 by immunofluorescence after 12 hours and 24 hours. Cell surface expression of Annexin V and PI exclusion is assayed to assess necrosis and apoptosis by flow cytometry analysis after 12 hours and 24 hours of incubation with clusterin. Adding clusterin blocks OPC differentiation with no impact on OPC viability.

### Example 7

### Factors Influencing Clusterin Expression by Glia

In the brain, clusterin is known to be expressed by astrocytes and recent data show that OPCs also express clusterin (Grubman et al., 2019). For this reason, primary astrocytes and OPCs are employed. Cells are isolated from newborn mice as described (Gaultier et al., 2009: Schildge et al., 2013; Fernandez-Castaneda et al., 2020). Cells are treated with increasing concentrations of apoptotic cells (Jurkat cells are employed as described in Fernandez-Castaneda et al., 2013), myelin debris (Gaultier et al., 2009), oligomeric Aβ, or increasing concentrations of cytokines known to be expressed in the brain of AD patients: TNFα, TGFβ, IL1β, IL 10 and IL-33 (Morimoto et al., 2011). Treatment with Vehicle are used as a negative control. Glia are treated for 2, 4, 6, and 12 hours and clusterin expression is by qPCR and immunoblot from both the cell lysate and conditioned media. Since preliminary data indicated that phagocytosis was involved in the upregulation of clusterin, these experiments are repeated for factors driving clusterin upregulation in the presence of Cytochalasin D (or vehicle) to block phagocytosis (Axline & Reaven, 1974; Soldati & Schliwa, 2006). Based on preliminary data, cytokines are not expected to drive clusterin expression. However, it is expected that apoptotic cells, myelin debris, and oligomeric Aβ increase clusterin expression. It is anticipated that this upregulation is blocked by Cytochalasin D, demonstrating that actin reorganization and perhaps phagocytosis is required. By comparing OPCs to astrocytes, the contribution of each cell type to the production of clusterin is investigated.

### Example 8

### Oligomeric Aβ Induced Expression of Clusterin In Vivo

Evidence presented here showed that clusterin expression was increased in AD and preclinical models of AD, such as the 5×FAD mouse line (see also Hong et al., 2013; Miners et al., 2017). If injection of oligomeric Aβ and myelin debris can drive localized expression of clusterin in the CNS is tested. Oligomeric Aβ and myelin debris is labeled with CypHer-5, a pH sensitive dye, to monitor phagocytosis (Fernandez-Castaneda et al., 2013). CypHer-5 fluorescence is activated by acidification in the lysosome, revealing active phagocytosis (Adie et al., 2003). Labeled Oligomeric Aβ and myelin debris is injected into the cortex and vehicle is injected in the contralateral cortex. Sections are prepared 12 hours and 24 hours post-injection and stained for clusterin and OPC (OLIG2, PDGFRα) or astrocyte marker (GFAP, ALDH1L1). Numbers of glia (OPCs and astrocytes) expressing clusterin and being positive for CypHer-5 fluorescence are quantified by immunofluorescence. In a preliminary experiment, it was shown that CypHer-5-Oligomeric Aβ injection induced a robust overexpression of clusterin 12 hours post injection: very little clusterin was detectable in the uninjected side (see FIG. 8A). Importantly it was demonstrated that OPCs can engulf labeled Oligomeric Aβ, as CypHer-5 staining colocalize with OPCs markers at the injection site (see FIG. 8B). It is hypothesized that Oligomeric Aβ and myelin debris drive clusterin expression in the mouse brain, potentially explaining the pathological expression of clusterin in AD and other neurological diseases such as multiple sclerosis.

### Example 9

### Identifying the Interactome of Clusterin in OPCs

The interactome of clusterin is investigated in order to identify its binding partners and discover the pathway involved in the blockade of OPC differentiation. OPCs plated in differentiating conditions are treated with recombinant clusterin as described in the preliminary data presented herein (see also FIG. 6). This recombinant protein contains an HIS-tag, allowing for easy purification using Ni-NTA-agarose beads. Clusterin is recovered from the protein extract and the conditioned media after 1 hour and 6 hours by affinity purification as previously described (Fernandez-Castaneda et al., 2013). Negative controls consist of OPCs treated with PBS. The composition of the protein interacting with clusterin is determined by mass spectrometry. Targets that have at least 3 unique peptides are selected. Interaction is confirmed by: (1) co-immunoprecipitation with a clusterin specific antibody; and (2) co-immunoprecipitation with commercial antibodies against the targets. Next, siRNA silencing is employed to test the impact of these interacting protein on OPC differentiation and viability. Novel regulators involved in the clusterin-mediated blockade of OPC differentiation and the molecular composition of the interactome of clusterin in OPCs are thus identified.

Based on the data presented herein, it is expected that clusterin blocks OPC differentiation and that clusterin expression is induced after treatment with debris (apoptotic cells, myelin debris, and oligomeric Aβ). If it is discovered that astrocytes produce more clusterin than OPCs, astrocytes are also tested. However, this would not change the overall hypothesis that pathogenic clusterin can block myelination, as clusterin is predominately a secreted protein and can affect OPCs in a non-cell-autonomous manner.

In the event that the proteomics approach does not identify new targets of clusterin, an unbiased approach to study clusterin biology is taken. Primary OPCs are treated with vehicle or clusterin for 6 hours and RNA expression is determined by RNAseq. Analysis of the expression differences across conditions reveals pathways that are controlled by clusterin during OPC differentiation and could also reveal insightful information about the mechanism of action of this protein.

### Introduction to Examples 10-13

Preliminary data showed that clusterin can block the differentiation of OPCs into oligodendrocytes and the subsequent upregulation of myelin markers. It was also demonstrated that clusterin was upregulated in the 5×FAD animal model of AD (see FIG. 5). To assess the impact of clusterin on oligodendrocytes and myelination in an animal model of Alzheimer's Disease, the role of clusterin on oligodendrocytes, myelin, and Aβ induced pathology is investigated using genetic and therapeutic approaches in the 5×FAD model.

The 5×FAD mouse is an animal model useful to study amyloid pathology that consists of overexpressing 5 mutations linked to familial AD present in the APP and PSEN1 genes. These mice are in the C57/BL6 background and commercially available (Jackson #34838). 5×FAD mice present with neurodegeneration (Oakley et al., 2006), Aβ plaques, memory deficits, and, importantly, present with myelin defects starting in young animals (1.5 months old; Gu et al., 2018), clusterin null mice are also available from Jackson (Cat #005642) on the C57/BL6 background. These two strains are crossed to generate 5×FAD Clu^{-/-} mice.

### Example 10

### Analysis of the Oligodendrocyte Lineage in the 5×FAD Model

5×FAD and 5×FAD Clu^{-/-} mice are employed. Sections of the CNS are prepared from animals prior to amyloid plaque deposition (1.5 months), after plaque deposition but before memory impairment (3 months), immediately following the onset of memory impairment (6 months), and at later stages of the disease (12 months) and stained with Olig2 and CC1 to monitor oligodendrocytes (Amram & Frenkel, 2017). Also included are C57/BL6 and Clu^{-/-} animals as additional controls. To quantify OPCs, sections are stained for PDGFR-α and Olig2 is (Fernandez-Castaneda et al., 2020). To quantify OPC proliferation and oligodendrocyte generation, daily BrdU injections are performed for 5 days prior to preparing samples as described in Aharoni et al., 2008. Tissue sections are then stained for BrdU, PDGFR-α, CC1, Olig2 and Nkx2.2 to examine cell proliferation and differentiation at any given time point. Two independent investigators, blinded to the status of clusterin, quantify all the parameters analyzed by histology. Based on preliminary data, it is anticipated that the overall number of oligodendrocytes and de novo generation of new oligodendrocytes is higher in the 5×FAD mice in the absence of clusterin.

### Example 11

### Myelin Analysis

To analyze the contribution of clusterin to myelin ultrastructure, myelin ultrastructure is analyzed in the corpus callosum using transmission electron microscopy (TEM) as described in Fernandez-Castaneda et al., 2020. Samples are prepared from the CNS of 1.5, 3, 6, and 12 month-old 5×FAD, 5×FAD Clu−/−, C56/BL6, and CLU^{-/-} mice. The g-ratio is determined from the samples as described (Orita et al., 2013). The number of myelinated versus non-myelinated axons, redundant myelin profiles, and the frequency of islands of cytoplasm within the otherwise compacted sheath are determined (Marcus et al., 2006). Redundant myelin and cytoplasmic islands are markers of immature myelin and/or compaction defects (Marcus et al., 2006). The structure of the nodes of Ranvier is analyzed. In particular, we nodal gap length, orientation of the paranodal loops, and integrity of the transverse bands are analyzed as described in Marcus et al., 2006 and Shepherd et al., 2012. It is postulated that the parameters analyzed show that myelin is preserved in 5×FAD mice in the absence in clusterin.

### Example 12

### Spatial Learning and Memory Assessment

The impact of clusterin on spatial learning and memory is investigated. The Morris water maze (MWM) is performed on 5-month-old 5×FAD and 5×FAD Clu−/− mice, an age where deficient learning and memory is present in the 5×FAD model (Gu et al., 2018). Briefly, each mouse undergoes five days of training, with four training trials per day, to assess how quickly they learn the location of a submerged platform. On the sixth day, spatial memory is assessed by removing the platform and evaluating the length of time each mouse spends in the quadrant previously containing the platform. Mice undergo the same six-day protocol with the platform in a different location to assess their ability to relearn a spatial task with new parameters (Vorhees & Williams, 2006). It is expected that 5×FAD mice lacking clusterin perform better in the MWM and present with better spatial learning and memory than control 5×FAD mice.

### Example 13

### Therapeutic Silencing of Clusterin

Clusterin is silenced in the CNS of 5×FAD mice using an AAV containing shRNA specific for mouse clusterin. AAV particles are purchased from Vector Biolabs ready to use and include GFP to monitor transduction in vivo. An AAV overexpressing scrambled shRNA and GFP is used as a negative control. To further test the therapeutic potential of clusterin, an antisense oligonucleotide (ASO) for clusterin is employed. There is currently an ASO targeting clusterin (Custirsen) in Phase 3 clinical trial for patients with prostate cancer that has been shown to safely and effectively reduce levels of clusterin (Chi et al., 2017). The use of the ASOs that have been already tested in patients could accelerate transition for bench to bedside. A scrambled ASO is used as a control. 5×FAD mice (4 months old) are injected with an ASO or AAV designed to decrease expression of clusterin or the corresponding control. For AAV experiments, mice are injected intravenously with 1×10¹¹ viral genomes per mouse. Gene expression is driven using the PHP.eB AAV serotype that effectively crosses the blood-brain barrier and infects CNS-resident cells (Mathiesen et al., 2020). For ASO treatment, 5×FAD mice receive 500 μg of a clusterin ASO every week for 4 weeks, a dosing paradigm used in previous clinical trials (Moore et al., 2017). Since the current design of antisense-oligonucleotides prevents them from crossing the blood brain barrier, ASOs are administered via intra-cistema magna injection as described in Dutra et al., 2018. The experiments described in EXAMPLES 10 and 12 are repeated to assess the impact of the treatment. It is hypothesized that therapeutic silencing of clusterin increases the number of oligodendrocytes as well as spatial learning and memory.

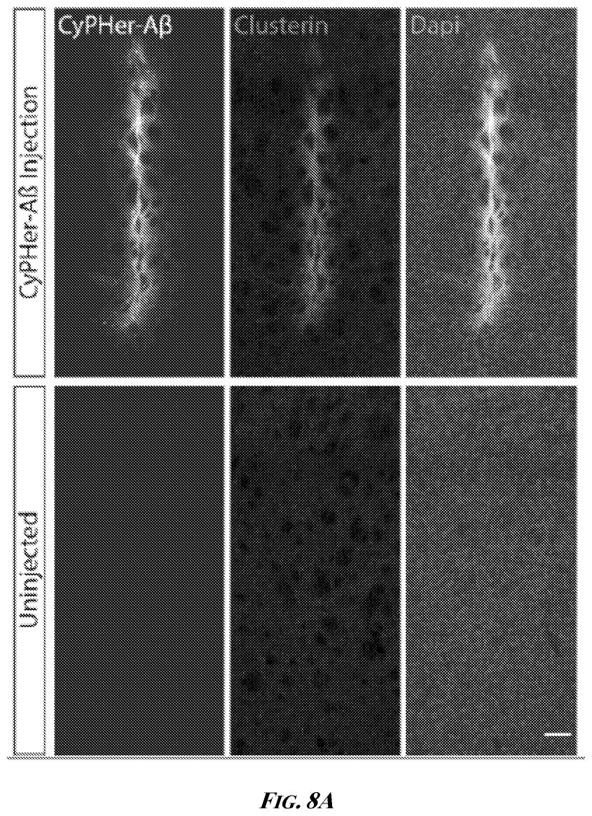

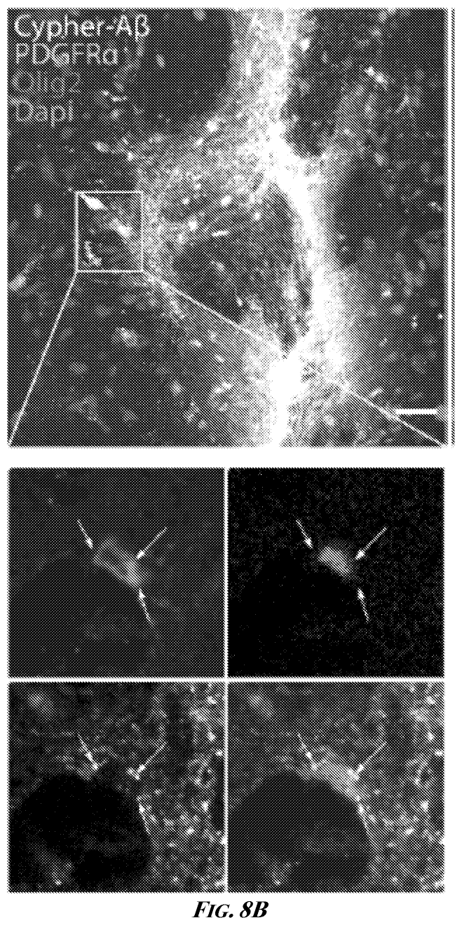

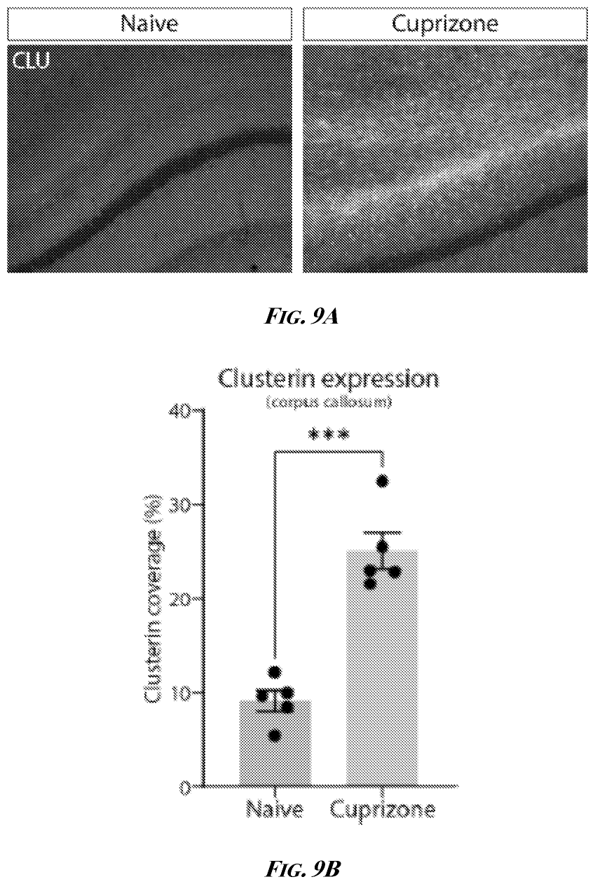

Based on preliminary data, it is anticipated that genetic and the therapeutic silencing of clusterin ameliorate the pathology observed in the 5×FAD mice. If a difference in the number of oligodendrocytes and myelination is undetected, the 5×FAD are tested with the cuprizone model of demyelination. Diet supplemented with cuprizone (5 weeks at 0.3% w/w) induces extensive demyelination of the medial corpus callosum (Doucette et al., 2010). However, demyelination induced in this manner is reversible, as remyelination occurs following the removal of the drug. Based on immunofluorescence in the corpus callosum, it has been observed that clusterin is upregulated during the Cuprizone model (see FIGS. 9A and 9B). The combination of the 5×FAD model, in which clusterin is highly upregulated, with Cuprizone, a model which normally results in the generation of new oligodendrocytes, could be an asset to study the impact of clusterin on the generation of oligodendrocytes. Sections of the medial corpus callosum (midline region above the for mix and between the lateral ventricles) are stained as proposed herein above to quantify OPCs and oligodendrocytes at the end of cuprizone treatment as well as 0.5, 1, 1.5, and 2 weeks following the removal of cuprizone, as described (see Fernandez-Castaneda et al., 2020).

### Introduction to Examples 14-16

### Quantifying the Number of OPCs and Oligodendrocytes in Normal Aging Vs AD Patients.

Increasing amounts of evidence indicate that AD patients present with altered myelin integrity, including a decrease in myelinated axons as well as a decrease in total myelin proteins (Roher et al., 2002: Desai et al., 2009; Desai et al., 2010; Zhan et al., 2014). Recent work has even proposed that myelin disruption precedes neurodegeneration in AD (Bartzokis, 2011). Despite the existence of these results suggesting that myelin pathology could be contributing to the disease, information about the abundance of oligodendrocyte lineage cells in AD is lacking.

The number of OPCs and oligodendrocytes in AD patients is precisely quantifyed and this quantification is compared to normal aging samples. Both the white matter and grey matter of the brain are examined. Furthermore, given the impact of clusterin on myelination reported in the data presented herein and its connection with AD, the level of expression is characterized and the cellular source of clusterin is determined.

### Example 14

### Quantifying Cells of the Oligodendrocyte Lineage in AD Patients

The cells of the oligodendrocyte lineage are quantified using RNAscope. RNAscope is an advanced In Situ Hybridization (ISH) method that allows multiplexing and has low background. This technique is well adapted for defining OPCs and Oligodendrocytes, as their identification requires two markers. RNAscope is performed using Olig2, Pdgfra and Mbp probes. OPCs are characterized by expression of Olig2 and Pdgfra and oligodendrocytes by expression Olig2 and Mbp. RNAscope was performed to detect OPCs (defined by co-expression of Olig2 and Pdgfra) in an AD brain sample (see FIG. 10). The results revealed that OPCs could be clearly identified with this method.

Based on the progression of AD pathology, the hippocampus (affected early), the frontal and/or temporal cortex (intermediate), and the occipital cortex (late stages) are examined (Prokop et al., 2019). For each brain area, gray and white matter are compared. Controls and three separate groups of AD patients with low, intermediate, and high pathological burden (10 per group) are examined as described in Prokop et al., 2019. Two independent investigators, blinded to the status of the specimens, quantify all the parameters analyzed by histology. It is anticipated that the number of oligodendrocytes are lower in AD vs normal aging. The number of OPCs are anticipated to be lower in AD patients.

### Example 15

Determining the Proportion of OPC Expressing Clusterin in AD Vs. Normal Aging with RNAscope

Preliminary data were consistent with the idea that pathological levels of clusterin can block OPC differentiation and impede myelin repair and maintenance in AD. It has also been shown that OPCs can express Clu in the mouse adult brain. In humans, a recent omics study reported that OPCs express clusterin under homeostatic conditions and upregulate its expression in AD (Grubman et al., 2019). Here, the 2 OPC clusters that have been identified in the described animal study (see FIGS. 2A-2D) are investigated. RNAscope is performed for Olig2, Pdgfra, Clu, and Gpr17 on normal aging specimen and late stage AD specimens (10 specimens/group). White and grey matters of the hippocampus, the frontal and/or temporal cortex, and the occipital cortex are examined (Prokop et al., 2019). The number of Clu positive and Gpr17 positive OPCs (defined by the expression of Olig2 and Pdgfra) in grey matter (cortex) and white matter are quantified. It is anticipated that the percentage of Clu positive OPCs is increased in AD samples when compared to normal aging specimen.

### Example 16

Quantifying Expression of Clusterin in AD Vs. Normal Aging with IHC

Clusterin has been shown to be increased in the brain, plasma, and cerebrospinal fluid of AD patients, as well as in the brains of AD mouse models (Hong et al., 2013: Miners et al., 2017). Because clusterin is a secreted protein, it is important to determine the site of production and the site of action. While RNAscope allows pinpointing of the cell type that produces the clusterin transcript, it does not permit determination of the site of action of clusterin. Here, IHC is combined with ISH to determine clusterin protein expression in the brain of AD patients compared to normal aging, and colocalization of clusterin expression with pathological hallmarks of AD.

Clusterin expression was detected in neurons and glial cells of samples from normal controls and AD patients. In AD patients, clusterin was notably increased (see FIG. 5A; see also McGeer et al., 1992; Kida et al., 1995; Harr et al., 1996). Furthermore, by combining IHC for Ab and ISH for clusterin, cells surrounding the AD plaques were identified that expressed clusterin transcripts (see FIG. 5B). The combination of IHC and ISH for clusterin further allowed for the partial discrepancy between CLU RNA expression and protein tissue localization to be determined, likely indicating that cells were secreting the protein into the local environment (see FIG. 5C).

First, IHC for clusterin is performed. Clusterin immunoreactivity is quantified by measuring area of coverage as described in Fernandez-Castaneda et al., 2020. Again, this experiment is performed with normal aging specimen (n=10) and late stage AD specimens (n=10) focusing on grey and white matters of the hippocampus, the frontal and/or temporal cortex, and the occipital cortex (Prokop et al., 2019). It is anticipated that clusterin immunoreactivity is higher in AD patients vs. normal aging.

Next, ISH for clusterin is combined with IHC for clusterin, Ab. Tau, and MBP to determine which site of AD pathology is associated with clusterin upregulation. Based on data presented herein, it is hypothesized that clusterin expression is denser around Ab plaques, as well as in areas of altered myelination.

### Example 17

### Clusterin is Expressed by a Subset of OPCs

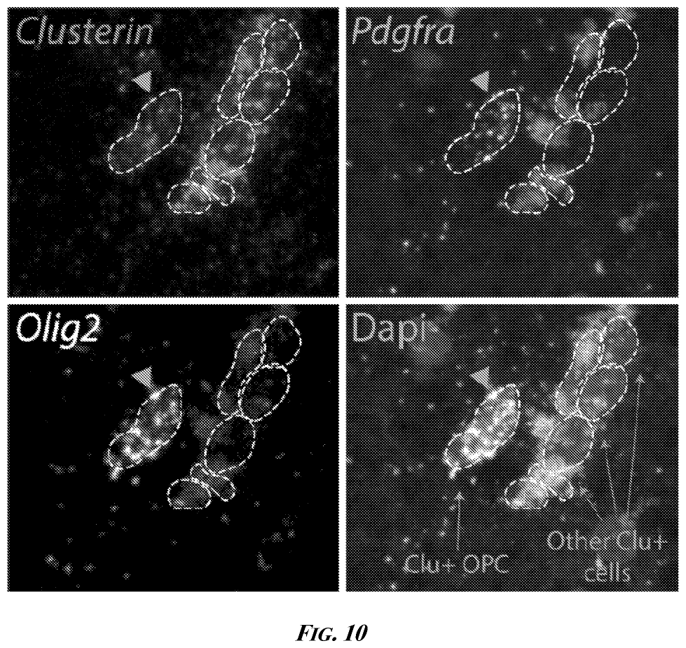

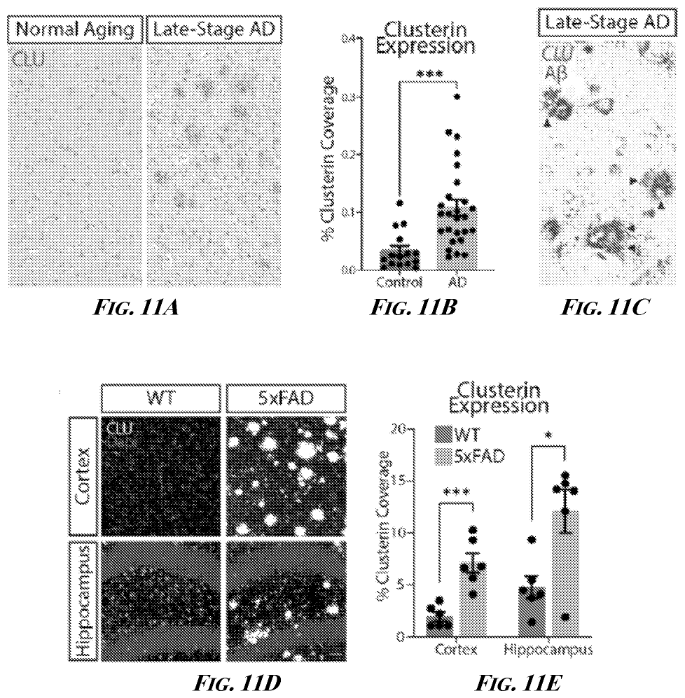

Clusterin, most commonly found as a secreted protein, is upregulated in the brains of patients with Alzheimer's disease (see FIGS. 11A and 11B. Interestingly, we found that cells expressing clusterin RNA could be found directly surrounding Aβ plaques (FIG. 11C). This observation was conserved in pre-clinical models of AD, as clusterin was also found to be upregulated in multiple brain regions of the 5×FAD mouse model of AD (FIGS. 11D and 11E). Because of the emerging role of OPCs and myelin in AD pathology, we investigated whether OPCs expressed clusterin. Using single cell sequencing in mice, we previously discovered the existence of two subsets of OPCs in adult mice. One cluster of OPCs (OPC1) was delineated by high clusterin expression compared to other cell types present in the dataset tested. Using RNAscope, we confirmed that a subset of OPCs expressed clusterin in vivo (FIGS. 11F and 11G). We used Gpr17 as a marker for the remaining subset of OPCs (OPC2) and used the same method to confirm that a subset of OPCs expressed Gpr17 in vivo (58.77%±3.19%).

We next asked if OPC1 and OPC2 were distinct from each other, or if cells expressed markers from both clusters at the same time. We used RNA-based flow cytometry (PrimeFlow) to demonstrate that a subset of Olig2+ cells expressed clusterin (OPC1), and a mutually exclusive population expressed Gpr17 (OPC2), with very few OPCs expressing detectable levels of both cluster markers (FIGS. 11H and 11I). This data confirmed the presence of the two distinct populations of OPCs we observed in our single-cell sequencing.

Finally, we also investigated whether human OPCs express clusterin. Surprisingly, we found that OPCs expressed clusterin both in the AD brain as well as in normal aging (FIG. 11J). These data showed that OPCs expressed clusterin in the adult CNS, confirming recent human single-cell sequencing data (Grubman et al., 2019). Furthermore, these results showed that clusterin, a risk factor for late-onset AD, was upregulated at the protein level in the parenchyma of AD patients.

### FIG. 18

### Phagocytosis of Oligomeric Aβ and Cellular Debris Results in Upregulation of Clusterin Expression by OPCs

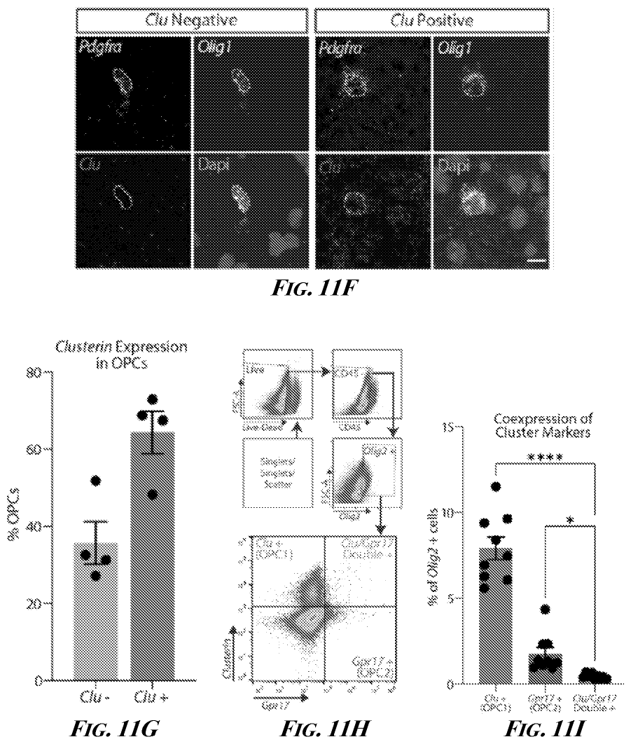

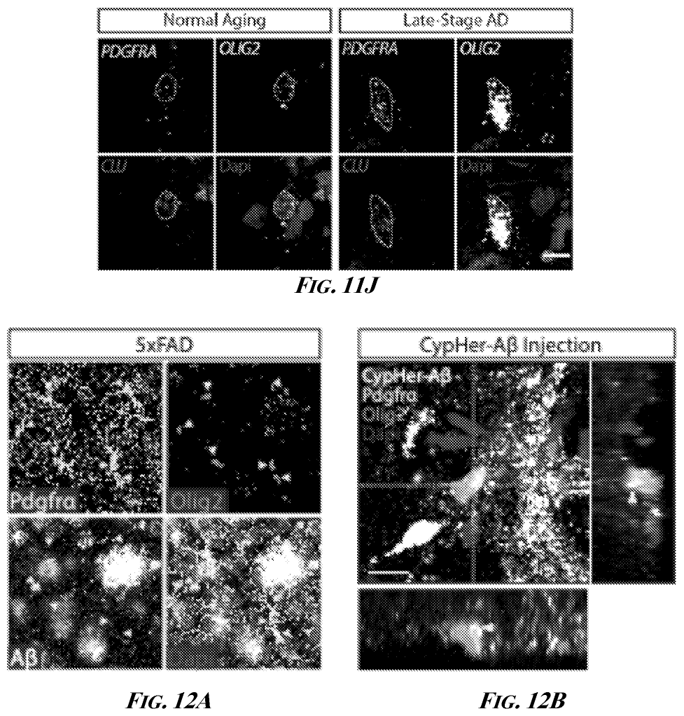

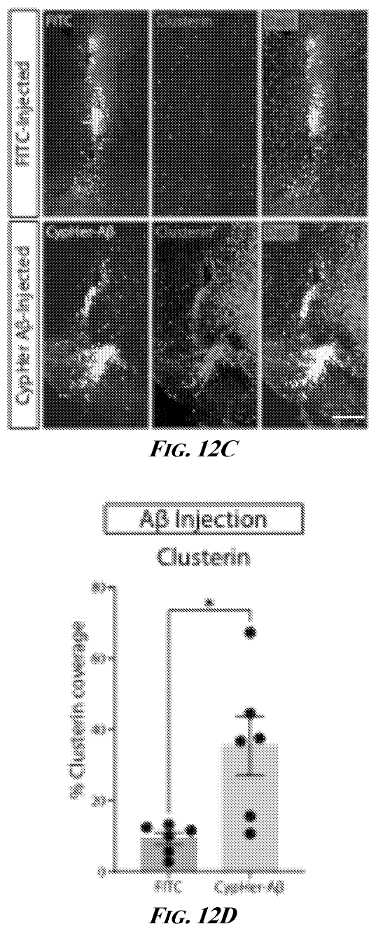

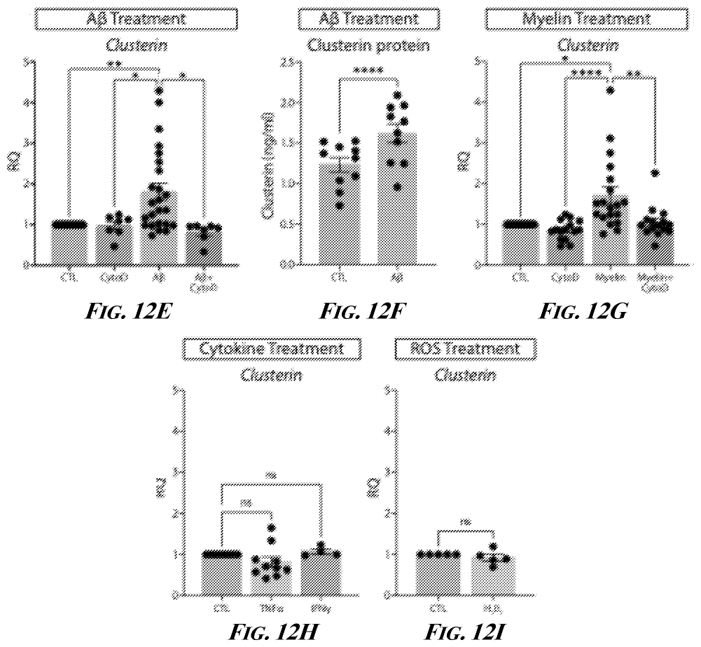

Given the data showing that OPCs express the AD-risk factor clusterin, we wanted to determine if OPCs could contribute to AD pathology by interacting with Aβ plaques. Using 5×FAD mice, we found that OPCs were surrounding and extending their processes into Aβ plaques (FIG. 12A). Because clusterin is known to facilitate debris clearance, we next wondered if OPCs were involved in the engulfment of Aβ oligomers in the brain. To test this, we injected WT mice with Aβ oligomers labeled with CypHer5e, a dye that only fluoresces when phagocytosed due to the acidic environment of the phagolysosome. We found that OPCs (and other cells) around the injection site phagocytosed Aβ within 12 hours of injection (FIG. 12B, an OPC was marked by co-expression of PDGFRα and Olig2, yellow arrowhead indicates co-localization of CypHer5e-Aβ). Surprisingly, we also found that Aβ injection also resulted in increased clusterin expression in the brain when compared to control protein injection (FIGS. 12C and 12D).

We next investigated whether Aβ treatment increases clusterin expression in OPCs. We treated primary OPCs in vitro with Aβ oligomers for 4 hours and observed a rapid increase in clusterin production at the RNA level (FIG. 12E). We subsequently confirmed that OPC treatment with Aβ also increased total cellular levels of the clusterin protein (FIG. 12F). We found that phagocytosis of Aβ was necessary to drive this upregulation of clusterin, as treatment with cytochalasin D (CytoD), an actin polymerization inhibitor that blocks phagocytosis, prevented Aβ from increasing clusterin production in OPCs (FIG. 12E). We noted that increase in clusterin expression was not specific to the phagocytic clearance of Aβ oligomers, as we also observed it when OPCs engulfed cellular debris. Treatment of OPCs with purified myelin debris or whole apoptotic cells also produced a similar upregulation of clusterin is that was abrogated by exposure to CytoD (FIG. 12G). In order to eliminate the possibility that clusterin upregulation was simply a response to any cellular stressor present in AD, we treated OPCs with other factors known to be upregulated in Alzheimer's disease, including TNFα, IFNγ, and reactive oxygen species (ROS), all of which failed to induce changes in clusterin expression (FIGS. 12H and 121).

In sum, these results indicated that OPC-mediated clearance of extracellular debris, including Aβ, was likely responsible for driving clusterin expression.

### Example 19

### Exogenous Clusterin Inhibits OPC Differentiation

The formation of new myelin has been increasingly recognized as a critical component of memory function. In fact, a recent study demonstrated that drugs that promote nascent myelin formation improve memory performance in a model of AD, highlighting the importance of identifying factors that prevent OPCs differentiation into oligodendrocytes in AD (Chen et al., 2021b). Several studies have reported that cellular debris and protein aggregates can prevent OPC differentiation, although the mechanism by which this occurs remains unclear (Kotter et al., 2006, Stoffels et al., 2013). These data, along with the observation that debris clearance drives clusterin expression in OPCs (see FIGS. 12E and 12F), made us question whether clusterin could inhibit OPC differentiation.

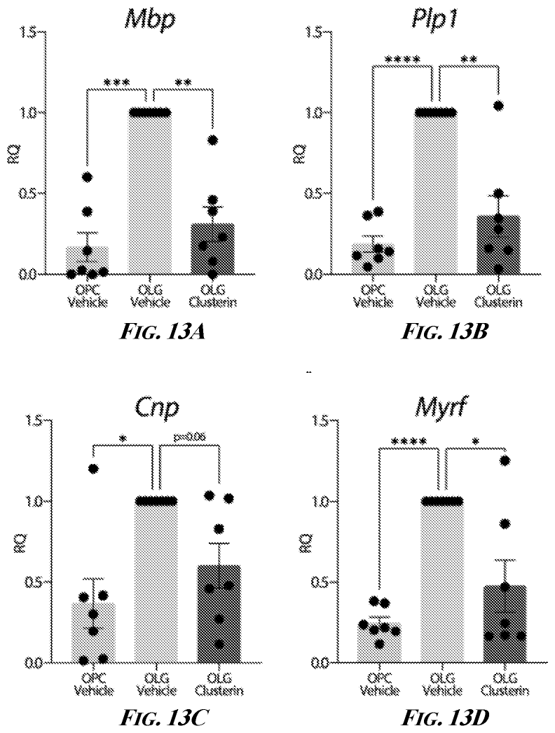

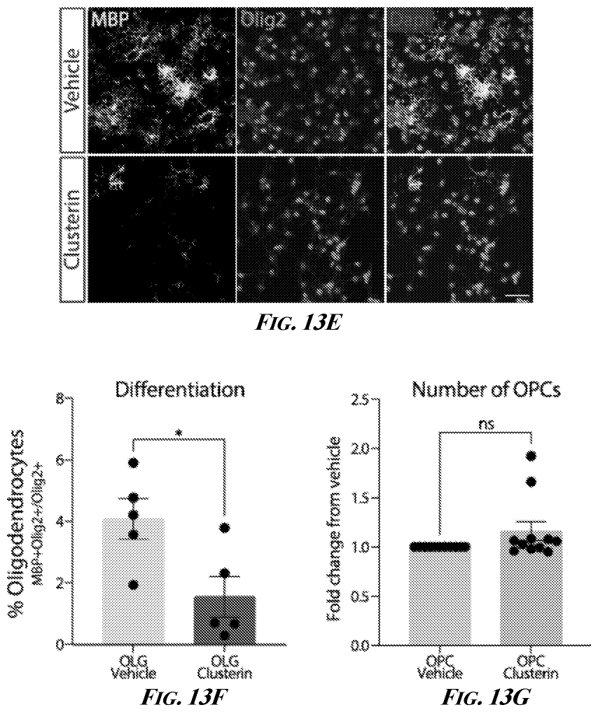

To address this question, we treated differentiating OPCs with exogenous clusterin and observed a striking decrease not only in genes encoding myelin proteins (Mbp, Pip1, Cnp), but also in Myrf, the master transcriptional regulator of the OPC differentiation program (FIGS. 13A-13D). We subsequently observed fewer MBP-positive oligodendrocytes when OPCs were differentiated in the presence of clusterin, when compared to the clusterin-free media (FIGS. 13E and 13F). Importantly, this decrease in differentiation was not due to OPC death (FIG. 13G). Overall, these data showed that clusterin is regulated by the phagocytosis of debris and acts as a potent inhibitor of OPC differentiation.

### Example 20

### Clusterin Inhibits Differentiation by Reducing IL-9 Production

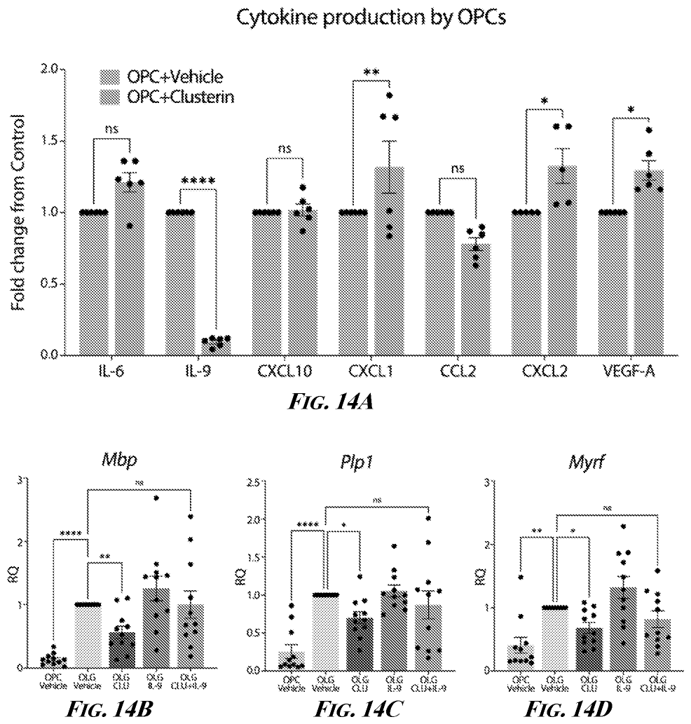

We next investigated what factors might be mediating clusterin's inhibition of OPC differentiation. OPCs ha^{v}e been shown to produce a variety of growth factors and cytokines that can significantly alter their local environment (Zhang et al., 2006; Kang et al., 2013a; Birey et al., 2015; Moyon et al., 2015; Wang et al., 2017). Additionally, clusterin has been shown to regulate to production of cytokines (Shim et al., 2011; Shim et al., 2012; Liang et al., 2021). Based on these data, we investigated whether clusterin could inhibit OPC differentiation by affecting growth factor and cytokine production. We performed a Luminex Assay on the supernatant of OPCs treated with vehicle or clusterin and found that clusterin altered secretion of multiple proteins (FIG. 14A). We noted a marked reduction in IL-9 secretion when OPCs were treated with clusterin (FIG. 14A).

IL-9 is a relatively understudied cytokine known to be produced by T-cells. To test if IL-9 plays a role in OPC differentiation, we added IL-9 to differentiating OPCs with or without clusterin. We found that adding exogenous IL-9 to differentiating OPCs prevented clusterin inhibition (FIGS. 14B-14D). As a control, we also tested if increase in VEGF keeps OPCs in an undifferentiated state, since VEGF has been shown to induce OPC proliferation. However, treatment of OPCs with a neutralizing anti-VEGF antibody failed to reduce clusterin-mediated inhibition of OPC differentiation. Overall, these data suggested that clusterin likely blocked differentiation of OPCs by inhibiting production of IL-9, and that IL-9 was an important factor in OPC differentiation.

### Example 21

### Identification of Clusterin Receptor(s) in OPCs

Clusterin receptors(s) are identified and the pathway involved in the blockade of OPC differentiation is characterized. OPCs plated in differentiating conditions are treated with recombinant His-tagged clusterin (His-clusterin), providing purification of His-clusterin and proteins bound to it using Ni-NTA-agarose beads, clusterin is recovered from the protein extract and the conditioned media after 1 hour and 6 hours by affinity purification as described in Fernandez-Castaneda et al., 2013. As a negative control, OPCs treated with PBS are employed. Proteins co-precipitating with clusterin are identified, in some embodiments by mass spectrometry. Targets that have at least three (3) unique peptides and that are described as cell surface proteins in the literature are selected. Interactions are confirmed by one or more of co-immunoprecipitation with a clusterin specific antibody and co-immunoprecipitation with commercial antibodies against the identified targets.

siRNA-mediated approaches are employed to test the impact of clusterin-interacting proteins on OPC differentiation and viability. Primary OPCs are isolated and transfected with siRNAs against the target, or a scrambled control, as described in de Faria et al., 2019. OPCs are switched to differentiation media to induce OPC maturation (see Fernandez-Castaneda et al., 2020). Maturation of OPCs into oligodendrocytes is followed by immunofluorescence analysis of OPC and oligodendrocyte markers after 3 days of culture. Markers are employed to confirm that cells are OPCs (PDGFR-α and OLIG2) or oligodendrocytes (CC1, CNP, PLP and MBP: see Baumann & Pham-Dinh, 2001). To ensure that any reduction in differentiation observed is not simply due to cell death. OPC apoptosis are monitored by staining for PDGFRα and cleaved caspase 3 by immunofluorescence after 24 hours and 72 hours. Cell surface expression of Annexin V is quantified and propidium iodide is employed to assess apoptosis and necrosis by flow cytometry after 24 hours and 72 hours of incubation with clusterin. Using this unbiased approach, novel receptors involved in the clusterin-mediated blockade of OPC differentiation are identified.

### Example 22

### The Role of IL-9 in OPC Differentiation

To identify whether clusterin can modify the composition of the OPC secretome, an unbiased multiplex analysis on the conditioned media of OPCs treated with vehicle or clusterin for 1-3 days was performed. It was discovered that clusterin treatment blocked IL-9 release from OPCs (see FIG. 14A). IL-9 is a growth factor for T-cell subsets 64, and IL-9 receptor is expressed by OPCs in vivo. However, production and secretion of IL-9 by OPCs has not been previously reported.

To test if clusterin-mediated IL-9 inhibition impacted OPC differentiation, OPCs were switched into differentiating conditions in the presence of recombinant clusterin (8 μg/ml) or vehicle (PBS)+/−IL-9 (100 ng/ml) for 3 days. Expression of Mbp, Plp, and Myrf, determined by qPCR, confirmed that exogenous clusterin blocks OPC differentiation, while the additional supplementation of IL-9 to clusterin-treated OPCs restored expression of these mature oligodendrocyte genes to a level similar as the untreated control (see FIGS. 14B-14D). These data suggested that clusterin could control secretion of IL-9, and that IL-9 was a critical factor for OPC differentiation.

The role of IL-9 in clusterin-mediated inhibition of OPC differentiation is further investigated. OPCs are switched to differentiation media to induce OPC maturation in the presence of vehicle or clusterin and with increasing concentrations of IL-9 (1-1000 ng/ml) or vehicle control. Blocking IL-9 is predicted to be detrimental to OPC differentiation.

To test this, OPCs are differentiated in the presence of a function blocking antibody against IL-9 (or isotype control). OPC differentiation and viability are evaluated as described herein.

Next, to confirm the role of IL-9 on OPC differentiation, IL-9 knockout mice in which endogenous IL-9 expression is inactivated by insertion of a YFP and a Cre downstream of the Il9 promoter are employed (Catalogue No. 031365: The Jackson Laboratory, Bar Harbor. Maine, United States of America). OPCs are prepared from C57BL6 or IL-9 KO mice on a C57BL/6J background and the viability and differentiation potential of these cells are assessed. It is anticipated that IL-9 treatment rescues OPC differentiation in the presence of exogenous clusterin and that IL-9 deficient OPCs are unable to differentiate as well as control cells.

### Example 23

### Analysis of the Oligodendrocyte Lineage in the 5×FAD Model

The 5×FAD mouse is an animal model useful in studying amyloid pathology that consists of overexpressing 5 mutations linked to familial AD present in the APP and PSEN1 genes. These mice are on the C57BL/6J background and are commercially available from The Jackson Laboratory (Catalogue No. 34838). 5×FAD mice present with neurodegeneration. Aβ plaques, memory deficits and, importantly, myelin defects. Clusterin knockout mice are also available from The Jackson Laboratory (Catalogue No. 005642) on the C57BL/6J background. These two strains are crossed to generate 5×FAD-Clu^{-/-} mice, which are used as follows.

Sections of the CNS are prepared from animals prior to amyloid plaque deposition (1.5 months), after plaque deposition but before memory impairment (3 months), immediately following the onset of memory impairment (6 months), and at later stages of the disease (12 months), and are stained with Olig2 and CC1 antibodies to monitor oligodendrocyte number. C57BL/6J and Clu^{-/-} animals are included as controls.

To quantify OPCs, sections are stained for PDGFR-α and Olig2. To quantify OPC proliferation and oligodendrocyte generation, daily BrdU injections are performed for 5 days prior to preparing samples. Tissue sections are stained for BrdU, PDGFR-α, CC1, Olig2, and Nkx2.2 to examine cell proliferation and differentiation. Two independent investigators, blinded to the status of clusterin, quantify all histology parameters. It is anticipated that the number of oligodendrocytes and the de novo generation of new oligodendrocytes (BrdU+ oligodendrocytes) are higher in the 5×FAD mice that do not express clusterin.

### Example 24

### Myelin Analysis

To analyze the effect of clusterin on myelin integrity, myelin ultrastructure in the corpus callosum is examined using transmission electron microscopy (TEM). Samples are prepared from the CNS of 1.5-, 3-, 6-, and 12-month-old 5×FAD, 5×FAD-Clu^{-/-}, C57BL/6J, and Clu^{-/-} animals. The g-ratio is determined from the samples as described in Orita et al., 2013. The number of myelinated versus non-myelinated axons, redundant myelin profiles, and the frequency of islands of cytoplasm within the otherwise compacted sheath are quantified. Redundant myelin and cytoplasmic islands are markers of immature myelin and/or compaction defects (Marcus et al., 2006). The structure of the nodes of Ranvier are also analyzed; in particular, nodal gap length, orientation of the paranodal loops, and integrity of the transverse bands are also analyzed. It is anticipated that the parameters analyzed show that myelin is preserved in 5×FAD mice in the absence in clusterin.

### Example 25

### Spatial Leamina and Memory Assessment

To investigate the impact of clusterin on spatial learning and memory, the Morris water maze (MWM) is performed on 5-month-old 5×FAD and 5×FAD-Clu^{-/-} mice, an age where deficient learning and memory is present in the 5×FAD model (Gu et al., 2018). Briefly, each mouse undergoes five days of training, with four training trials per day, to assess how quickly it learns the location of a submerged platform. On the sixth day, spatial memory is assessed by removing the platform and evaluating the length of time each mouse spends in the quadrant previously containing the platform. Mice then undergo the same six-day protocol with the platform in a different location to assess their ability to relearn a spatial task with new parameters. It is anticipated that 5×FAD mice lacking clusterin perform better in the MWM and present with better spatial learning and memory than control 5×FAD mice.

### Example 26

### Therapeutic Silencing of Clusterin and IL-9 Overexpression

Clusterin is silenced in the CNS of 5×FAD mice using an AAV containing shRNA specific for mouse clusterin. Additionally, the effects of increasing IL-9 on 5×FAD mice is tested using an AAV to overexpress this cytokine. AAV particles are purchased from Vector Biolabs (Malvern, Pennsylvania, United States of America) ready to use and GFP is included to monitor transduction in vivo. An AAV overexpressing scrambled shRNA and GFP is used as the clusterin negative control, and an AAV expressing GFP alone is used as a negative control for the IL-9 experiments.

To further test the therapeutic potential of clusterin, an antisense oligonucleotide (ASO) for clusterin is employed. There is currently an ASO targeting clusterin (Custirsen) in Phase 3 clinical trial in the United States for patients with prostate cancer that has been shown to safely and effectively reduce levels of clusterin (Chi et al., 2017). The use of an ASO that has already been tested in patients could accelerate the bench to bedside transition. A scrambled ASO is used as a control. 5×FAD mice at 4 months old are injected with an ASO or AAV treatments or the corresponding control. For AAV experiments, mice are injected intravenously with 1×10¹¹ viral genomes per mouse. Gene expression is driven using the PHP.eB AAV serotype that effectively crosses the blood-brain barrier and infects CNS-resident cells (Mathiesen et al., 2020). For ASO treatment, 5×FAD mice receive 500 μg of a clusterin ASO every week for 4 weeks, a dosing paradigm used in previous clinical trials (Moore et al., 2017). Since the current design of antisense-oligonucleotides prevents them from crossing the blood brain barrier, ASOs are administered via intra-cistema magna injection as described in Dutra et al., 2018. These experiments are performed to assess the impact of the treatment. It is anticipated that therapeutic silencing of clusterin and IL-9 overexpression increases the number of oligodendrocytes as well as spatial learning and memory.

### REFERENCES

All references cited in the instant disclosure (indicated by bracketed numbers) as well as those listed below, including but not limited to all patents, patent applications and publications thereof, scientific journal articles, and database entries (such as but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Adie et al. (2003) CypHer 5: a generic approach for measuring the activation and trafficking of G protein-coupled receptors in live cells. Assay Drug Dev Technol 1:251-259.
Aghajanian et al. (2017) PDGFRα functions in endothelial-derived cells to regulate neural crest cells and the development of the great arteries. Dis Model Mech 10:1101-1108.
Aharoni et al. (2008) Demyelination arrest and remyelination induced by glatiramer acetate treatment of experimental autoimmune encephalomyelitis. Proc Natl Acad Sci USA 105:11358-11363.
Ahmad et al. (2017) Oxidative toxicity in diabetes and Alzheimer's disease: mechanisms behind ROS/RNS generation. J Biomed Sci 24:76.
Altschul et al. (1990a) Basic local alignment search tool. J Mol Biol 215:403-410.
Altschul et al. (1990b) Protein database searches for multiple alignments. Proc Natl Acad Sci is USA 87:14:5509-13.
Altschul et al. (1997) Gapped BLAST and PSI-BLAST: a new generation of protein database search programs. Nucleic Acids Res 25:3389-3402.
Amram & Frenkel (2017) “Animal Models of Alzheimer's Disease”, in Neuroprotection in Alzheimer's Disease, Gozes (ed.). Elsevier/Academic Press, Boston, Massachusetts, United States of America, Chapter 3, pages 31-58.
Axline & Reaven (1974) Inhibition of phagocytosis and plasma membrane mobility of the cultivated macrophage by cytochalasin B. Role of subplasmalemmal microfilaments. J Cell Biol 62:647-659.
Bartzokis (2011) Alzheimer's disease as homeostatic responses to age-related myelin breakdown. Neurobiol Aging 32:1341-1371.
Baumann & Pham-Dinh (2001) Biology of oligodendrocyte and myelin in the mammalian central nervous system. Physiol Rev 81:871-927.
Baxi et al. (2014) A selective thyroid hormone beta receptor agonist enhances human and rodent oligodendrocyte differentiation. Glia 62:1513-1529.
Bertram & Tanzi (2009) Genome-wide association studies in Alzheimer's disease. Hum Mol Genet 18:R137-R145.
Bertram et al. (2007) Systematic meta-analyses of Alzheimer disease genetic association studies: the AlzGene database. Nat Genet 39:17-23.
Bird et al. (1988) Single-chain antigen-binding proteins. Science 242:423-426.
Birey et al. (2015) Genetic and Stress-Induced Loss of NG2 Glia Triggers Emergence of Depressive-like Behaviors through Reduced Secretion of FGF2. Neuron 88:941-956.
Bondjers et al. (2003) Transcription profiling of platelet-derived growth factor-B-deficient mouse embryos identifies RGS5 as a novel marker for pericytes and vascular smooth muscle cells. Am J Pathol 162:721-729.
Braskie et al. (2011) Common Alzheimer's disease risk variant within the CLU gene affects white matter microstructure in young adults. J Neurosci 31:6764-6770.
Butler et al. (2018) Integrating single-cell transcriptomic data across different conditions, technologies, and species. Nat Biotechnol 36:411-420.
Chacon-De-La-Rocha et al. (2020) Accelerated Dystrophy and Decay of Oligodendrocyte Precursor Cells in the APP/PSI Model of Alzheimer's-Like Pathology. Front Cell Neurosci 14:575082.
Chang et al. (2017) Tumor necrosis factor alpha Inhibition for Alzheimer's Disease. J Cent Nerv Syst Dis 9:1179573517709278.
Chen et al. (2021a) Clusterin secreted from astrocyte promotes excitatory synaptic transmission and ameliorates Alzheimer's disease neuropathology. Mol Neurodegener 16:5.
Chen et al. (2021b) Enhancing myelin renewal reverses cognitive dysfunction in a murine model of Alzheimer's disease. Neuron 14:2292-2307.
Chi et al. (2017) Custirsen in combination with docetaxel and prednisone for patients with metastatic castration-resistant prostate cancer (SYNERGY trial): a phase 3, multicentre, open-label, randomised trial. Lancet Oncol 18:473-485.
Clark et al. (1995) Cell lines for the production of recombinant adeno-associated virus. Hum Gene Ther 6:1329-1341.
Davidson et al. (1993) A model system for in vivo gene transfer into the central nervous system using an adenoviral vector. Nat Genet 3:219-223.
de Faria et al. (2019) TMEM10 Promotes Oligodendrocyte Differentiation and is Expressed by Oligodendrocytes in Human Remyelinating Multiple Sclerosis Plaques. Sci Rep 9:3606.
Dean et al. (2017) Association of Amyloid Pathology With Myelin Alteration in Preclinical Alzheimer Disease. JAMA Neurol 74:41-49.
DeMattos et al. (2001) Purification and characterization of astrocyte-secreted apolipoprotein E and J-containing lipoproteins from wild-type and human apoE transgenic mice. Neurochem Int 39:415-425.

Desai et al. (2009) Triple-transgenic Alzheimer's disease mice exhibit region-specific abnormalities in brain myelination patterns prior to appearance of amyloid and tau pathology. Glia 57:54-65.
Desai et al. (2010) Early Oligodendrocyte/Myelin Pathology in Alzheimer's Disease Mice Constitutes a Novel Therapeutic Target. Am J Pathol 177:1422-1435.
Deshmane et al. (2009) Monocyte chemoattractant protein-1 (MCP-1): an overview. J Interferon Cytokine Res 29:313-326.
Dimou et al. (2008) Progeny of Olig2-expressing progenitors in the gray and white matter of the adult mouse cerebral cortex. J Neurosci 28:10434-10442.
Doucette et al. (2010) Age-related and cuprizone-induced changes in myelin and transcription factor gene expression and in oligodendrocyte cell densities in the rostral corpus callosum of mice. Cell Mol Neurobiol 30:607-629.
Dutra et al. (2018) Protective Role of UCP2 in Oxidative Stress and Apoptosis during the Silent Phase of an Experimental Model of Epilepsy Induced by Pilocarpine. Oxid Med Cell Longev 2018:6736721.
Endale et al. (2017) Temporal, spatial, and phenotypical changes of PDGFRα expressing fibroblasts during late lung development. Dev Biol 425:161-175.
Erben et al. (2018) A Novel Ultrasensitive In Situ Hybridization Approach to Detect Short Sequences and Splice Variants with Cellular Resolution. Mol Neurobiol 55:6169-6181.
Falcao et al. (2018) Disease-specific oligodendrocyte lineage cells arise in multiple sclerosis. Nat Med 24:1837-1844.
Falgarone & Chiocchia (2009) Chapter 8: Clusterin: A multifacet protein at the crossroad of inflammation and autoimmunity. Adv Cancer Res 104:139-170.
Fernandez-Castaneda & Gaultier (2016) Adult oligodendrocyte progenitor cells—Multifaceted regulators of the CNS in health and disease. Brain Behav Immun 57:1-7.
Fernandez-Castaneda et al. (2013) Identification of the low density lipoprotein (LDL) receptor-related protein-1 interactome in central nervous system myelin suggests a role in the clearance of necrotic cell debris. J Biol Chem 288:45384548.
Fernandez-Castaneda et al. (2020) The active contribution of OPCs to neuroinflammation is mediated by LRP1. Acta Neuropathol 139:365-382.
Franco et al. (2015) Optimizing culture medium composition to improve oligodendrocyte progenitor cell yields in vitro from subventricular zone-derived neural progenitor cell neurospheres. PLoS One 10:e0121774.
Fumagalli et al. (2011) Phenotypic changes, signaling pathway, and functional correlates of GPR17-expressing neural precursor cells during oligodendrocyte differentiation. J Biol Chem 286:10593-10604.
Gait (1985) Oligonucleotide Synthesis: A Practical Approach. IRL Press, Oxford, England.
Gao et al. (1998) High-titer adeno-associated viral vectors from a Rep/Cap cell line and hybrid shuttle virus. Gene Ther 9:2353-2362.
Gaultier et al. (2009) Low-density lipoprotein receptor-related protein 1 is an essential receptor for myelin phagocytosis. J Cell Sci 122:1155-1162.
Geller et al. (1990) Infection of cultured central nervous system neurons with a defective herpes simplex virus 1 vector results in stable expression of Escherichia coli beta-galactosidase. Proc Natl. Acad. Sci USA 87:1149-1153.
Geller et al. (1993) Long-term increases in neurotransmitter release from neuronal cells expressing a constitutively active adenylate cyclase from a herpes simplex virus type 1 vector. Proc Natl Acad Sci USA 90:7603-7607.
Geller et al. (1995) An HSV-1 vector expressing tyrosine hydroxylase causes production and release of L-dopa from cultured rat striatal cells. J Neurochem 64:487-496.
Genaro (ed.) (1985) Remington's Pharmaceutical Sciences, Mack Publishing Co., Easton, Pennsylvania, United States of America.
Green & Sambrook et al. (2012) Molecular Cloning: A Laboratory Manual (Fourth Edition). Cold Spring Harbor Laboratory Press, Cold Spring Harbor, New York, United States of America.
Griffiths-Jones (2004) The microRNA Registry. Nucleic Acids Research 32(suppl_1):D109-D111.
Grimm et al. (1998) Novel tools for production and purification of recombinant adenoassociated virus vectors. Hum Gene Ther 9:2745-2760.
Gross & Mienhofer (eds.) (1981) The Peptides, Vol. 3, Academic Press, New York, New York, pages 3-88.
Grubman et al. (2019) A single-cell atlas of entorhinal cortex from individuals with Alzheimer's disease reveals cell-type-specific gene expression regulation. Nat Neurosci 22:2087-2097.
Gu et al. (2011) Association of Clusterin Gene Polymorphisms with Late-Onset Alzheimer's Disease. Dement Geriatr Cogn Disord 32:198-201.
Gu et al. (2018) Myelin changes at the early stage of 5×FAD mice. Brain Res Bull 137:285-293.
Hall et al. (1996) Spinal cord oligodendrocytes develop from ventrally derived progenitor cells that express PDGF alpha-receptors. Development 122:4085-4094.
Harlow et al. (1989) Antibodies: A Laboratory Manual. Cold Spring Harbor Laboratory Press, Cold Spring Harbor, New York, United States of America.
Harlow et al. (1999) Using Antibodies: A Laboratory Manual, Cold Spring Harbor Laboratory Press, Cold Spring Harbor. New York, United States of America.
Harold et al. (2009) Genome-wide association study identifies variants at CLU and PICALM associated with Alzheimer's disease. Nat Genet 41:1088-1093.
Harr et al. (1996) Brain expression of apolipoproteins E, J. and A-I in Alzheimer's disease. J Neurochem 66:2429-2435.
Hong et al. (2013) Quantitative proteomic analysis of the hippocampus in the 5×FAD mouse model at early stages of Alzheimer's disease pathology. J Alzheimers Dis 36:321-334.
Huang et al. (2020) Clinical trials of new drugs for Alzheimer disease. J Biomed Sci 27:18-18.
Huston et al. (1988) Protein engineering of antibody binding sites: recovery of specific activity in an anti-digoxin single-chain Fv analogue produced in Escherichia coli. Proc Natl Acad Sci USA 85:5879-5883.
Inoue & Russell (1998) Packaging cells based on inducible gene amplification for the production of adeno-associated virus vectors. J Virol 72:7024-7031.
Kang et al. (2010) NG2+CNS glial progenitors remain committed to the oligodendrocyte lineage in postnatal life and following neurodegeneration. Neuron 68:668-681.
Kang et al. (2013a) Actl mediates IL-17-induced EAE pathogenesis selectively in NG2+ glial cells. Nat Neurosci 16:1401-1408.
Kang et al. (2013b) Degeneration and impaired regeneration of gray matter oligodendrocytes in amyotrophic lateral sclerosis. Nat Neurosci 16:571-579.
Kaplitt et al. (1994) Long-term gene expression and phenotypic correction using adeno-associated virus vectors in the mammalian brain. Nat Genet 8:148-154.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Kida et al. (1995) Deposition of apolipoproteins E and J in senile plaques is topographically determined in both Alzheimer's disease and Down's syndrome brain. Brain Res 685:211-216.
Kirby et al. (2019) Oligodendrocyte precursor cells present antigen and are cytotoxic targets in inflammatory demyelination. Nat Comm 10:3887.
Kotter et al. (2006) Myelin impairs CNS remyelination by inhibiting oligodendrocyte precursor cell differentiation. J Neurosci 26:328-332.
Kurihara et al. (2010) A putrescine-inducible pathway comprising PuuEYneI in which gamma-aminobutyrate is degraded into succinate in Escherichia coli K-12. J Bacteriol 192:4582-4591.
Lambert et al. (2009) Genome-wide association study identifies variants at CLU and CR1 associated with Alzheimer's disease. Nat Genet 41(10):1094-1099.
Lamoureux et al. (2011) Clusterin Inhibition Using OGX-011 Synergistically Enhances Hsp90 Inhibitor Activity by Suppressing the Heat Shock Response in Castrate-Resistant Prostate Cancer. Cancer Res 71:5838-5849.
Larbig et al. (2007) Screening for Inhibitors of Tau Protein Aggregation into Alzheimer Paired Helical Filaments: A Ligand Based Approach Results in Successful Scaffold Hopping. Current Alzheimer Research 4:315-323.
LeGal LaSalle et al. (1993) An adenovirus vector for gene transfer into neurons and glia in the brain. Science, 259:988-990.
Li et al. (2018) PDGFRA marks a cellular lineage with distinct contributions to myofibroblasts in lung maturation and injury response, eLife 7:e36865.
Liang et al. (2021) Role of Clusterin/NF-κB in the secretion of senescence-associated secretory phenotype in Cr(VI)-induced premature senescent L-02 hepatocytes. Ecotoxicol Environ Saf 219:112343.
Lieber et al. (1999) Integrating adenovirus-adeno-associated virus hybrid vectors devoid of all viral genes. J Virol 73:9314-9324.
Lim et al. (1995) in DNA Cloning: Mammalian Systems, Glover (ed.), Oxford Univiversity Press, Oxford, England.
Liu et al. (1999) Production of recombinant adeno-associated virus vectors using a packaging cell line and a hybrid recombinant adenovirus. Gene Ther 6:293-299.
Lu et al. (2000) Sonic Hedgehog-Regulated Oligodendrocyte Lineage Genes Encoding bHLH Proteins in the Mammalian Central Nervous System. Neuron 25:317-329.
Marcus et al. (2006) Sulfatide is essential for the maintenance of CNS myelin and axon structure. Glia 53:372-381.
Marques et al. (2016) Oligodendrocyte heterogeneity in the mouse juvenile and adult central nervous system. Science 352:1326-1329.
Marques et al. (2018) Transcriptional Convergence of Oligodendrocyte Lineage Progenitors during Development. Dev Cell 46:504-517.
Mathiesen et al. (2020) CNS Transduction Benefits of AAV-PHP.eB over AAV9 Are Dependent on Administration Route and Mouse Strain. Mol Ther Methods Clin Dev 19:447-458.
Matsushita et al. (1998) Adeno-associated virus vectors can be efficiently produced without helper virus. Gene Ther 5:938-945.
Maurice & Su (2009) The pharmacology of sigma-1 receptors. Pharmacol Ther 124:195-206.
May et al. (2011) Robust central reduction of amyloid-β in humans with an orally available, non-peptidic β-secretase inhibitor. J Neurosci 31(46):16507-16516.
McGeer et al. (1992) Distribution of clusterin in Alzheimer brain tissue. Brain Res 579:337-341.
Miners et al. (2017) Clusterin levels are increased in Alzheimer's disease and influence the regional distribution of Abeta. Brain Pathol 27:305-313.
Moore et al. (2017) Evaluation of Antisense Oligonucleotides Targeting ATXN3 in SCA3 Mouse Models. Mol Ther Nucleic Acids 7:200-210.
Morimoto et al. (2011) Expression profiles of cytokines in the brains of Alzheimer's disease (AD) patients compared to the brains of non-demented patients with and without increasing AD pathology. J Alzheimers Dis 25:59-76.
Moyon et al. (2015) Demyelination causes adult CNS progenitors to revert to an immature state and express immune cues that support their migration. J Neurosci 35:4-20.
Mullan et al. (2013) Plasma clusterin levels and the rs1 1136000 genotype in individuals with mild cognitive impairment and Alzheimer's disease. Curr Alzheimer Res 10:973-978.
Murphy et al. (1988) SP-40,40, a newly identified normal human serum protein found in the SC5b-9 complex of complement and in the immune deposits in glomerulonephritis. J Clin Invest 81:1858-1864.
Muzyczka (1992) Use of adeno-associated virus as a general transduction vector for mammalian cells. Curr Topics Microbiol Immunol 158:97-129.
Necula et al. (2005) Cyanine Dye N744 Inhibits Tau Fibrillization by Blocking Filament Extension: Implications for the Treatment of Tauopathic Neurodegenerative Diseases. Biochemistry 44:10227-10237.
Nishiyama et al. (1996) Co-localization of NG2 proteoglycan and PDGF alpha-receptor on O2A progenitor cells in the developing rat brain. J Neurosci Res 43:299-314.
Oakley et al. (2006) Intraneuronal beta-amyloid aggregates, neurodegeneration, and neuron loss in transgenic mice with five familial Alzheimer's disease mutations: potential factors in amyloid plaque formation. J Neurosci 26:10129-10140.
Orita et al. (2013) Schwann cell LRP1 regulates remak bundle ultrastructure and axonal interactions to prevent neuropathic pain. J Neurosci 33:5590-5602.
Pan et al. (2020) Preservation of a remote fear memory requires new myelin formation. Nature Neuroscience 23:487-499.
PCT International Patent Application Publication No. WO 2020/185676.
Pereira et al. (2018) Protective molecular mechanisms of clusterin against apoptosis in cardiomyocytes. Heart Fail Rev 23:123-129.
Pickhardt et al. (2005) Screening for Inhibitors of Tau Polymerization. Current Alzheimer Research 2:219-226.
Prokop et al. (2019) Impact of TREM2 risk variants on brain region-specific immune activation and plaque microenvironment in Alzheimers disease patient brain samples. Acta Neuropathol 138:613-630.
Quantin et al. (1992) Adenovirus as an expression vector in muscle cells in vivo. Proc Nat!Acad Sci USA 89:2581-2584.
Rivers et al. (2008) PDGFRA/NG2 glia generate myelinating oligodendrocytes and piriform projection neurons in adult mice. Nat Neurosci 11:1392-1401.
Roher et al. (2002) Increased AD Peptides and Reduced Cholesterol and Myelin Proteins Characterize White Matter Degeneration in Alzheimer's Disease. Biochemistry 41:11080-11090.
Rosenfeld et al. (1992) In vivo transfer of the human cystic fibrosis transmembrane conductance regulator gene to the airway epithelium. Cell 68:143-155.
Sakry et al. (2014) Oligodendrocyte precursor cells modulate the neuronal network by activity-dependent ectodomain cleavage of glial NG2. PLoS Biol 12:e1001993.
Sanjana et al. (2014) Improved vectors and genome-wide libraries for CRISPR screening. Nat Methods 11(8):783-784.
Schildge et al. (2013) Isolation and culture of mouse cortical astrocytes. J Vis Exp 71:50079.
Sengupta et al. (2016) The Role of Amyloid-beta Oligomers in Toxicity, Propagation, and Immunotherapy. EBioMedicine 6:4249.
Seo et al. (2013) Oligodendrocyte precursors induce early blood-brain barrier opening after white matter injury. J Clin Invest 123:782-786.
Shepherd et al. (2012) Paranodal reorganization results in the depletion of transverse bands in the aged central nervous system. Neurobiol Aging 33:203 e213-224.
Shim et al. (2011) Clusterin induces matrix metalloproteinase-9 expression via ERK1/2 and PI3K/Akt/NF-κB pathways in monocytes/macrophages. J Leukoc Biol 90:761-769.
Shim et al. (2012) Clusterin induces the secretion of TNF-α and the chemotactic migration of macrophages. Biochem Bioph Res Comm 422:200-205.
Simon et al. (2011) Progenitors in the adult cerebral cortex: cell cycle properties and regulation by physiological stimuli and injury. Glia 59:869-881.
Sims & Smith (2010) The IL-1 family: regulators of immunity. Nature Reviews. Immunology 10:89-102.
Smyth et al. (2018) Markers for human brain pericytes and smooth muscle cells. J Chem Neuroanat 92:48-60.
Soldati & Schliwa (2006) Powering membrane traffic in endocytosis and recycling. Nat Rev Mol Cell Biol 7:897-908.
Steadman et al. (2020) Disruption of Oligodendrogenesis Impairs Memory Consolidation in Adult Mice. Neuron 105:150-164.e156.
Stoffels et al. (2013) Fibronectin aggregation in multiple sclerosis lesions impairs remyelination. Brain 136:116-131.
Stratford-Perricadet et al. (1992) Widespread long-term gene transfer to mouse skeletal muscles and heart. J Clin Invest 90:626-630.
Stuart et al. (2019) Comprehensive Integration of Single-Cell Data. Cell 177:1888-1902 e1821.
Tamayose et al. (1996) A new strategy for large-scale preparation of high-titer recombinant adeno-associated virus vectors by using packaging cell lines and sulfonated cellulose column chromatography. Hum Gene Ther 7:507-513.
Taniguchi et al. (2005a) Effects of different anti-tau antibodies on tau fibrillogenesis: RTA-1 and RTA-2 counteract tau aggregation. FEBS Letters 579:1399-1404.
Taniguchi et al. (2005b) Inhibition of Heparin-induced Tau Filament Formation by Phenothiazines, Polyphenols, and Porphyrins. The Journal of Biological Chemistry 280:7614-7623.
Thambisetty et al. (2010) Association of plasma clusterin concentration with severity, pathology, and progression in Alzheimer disease. Arch Gen Psychiatry 67:739-748.
U.S. Patent Application Publication Nos. 2004/0009949; 2008/0206812; 2009/0209755; 2014/0189896; 2014/0242664; 2014/0287838; 2014/0357530; 2016/0024494; 2016/0362688; 2017/0157267; 2018/0177790; 2018/0311290; 2019/0000991; 2019/0008909; 2019/0002916; 2019/0048362; 2019/0060489; 2020/0063102; 2020/0071699.
U.S. Pat. Nos. 4,895,841; 4,948,807; 6,099,863; 6,316,023; 6,335,031; 6,358,527; 6,506,559; 7,056,704; 7,160,559; 7,592,441; 7,727,548; 7,727,552; 7,825,229; 7,825,230; 7,960,359; 8,039,009; 8,039,009; 8,058,291; 8,168,209; 8,173,708; 8,173,708; 8,283,379; 8,283,379; 8,329,752; 8,329,752; 8,338,486; 8,362,085; 8,362,085; 8,372,968; 8,420,391; 8,598,233; 8,697,359; 8,809,058; 9,365,647; 9,512,211; 9,540,659; 9,701,984; 9,840,719; 10,214,572; 10,392,632.
van den Brink et al. (2017) Single-cell sequencing reveals dissociation-induced gene expression in tissue subpopulations. Nat Methods 14:935-936.
Vanzulli et al. (2020) Disruption of oligodendrocyte progenitor cells is an early sign of pathology in the triple transgenic mouse model of Alzheimer's disease. Neurobiol Aging 94:130-139.
Vigano et al. (2016) GPR17 expressing NG2-Glia: Oligodendrocyte progenitors serving as a reserve pool after injury. Glia 64:287-299.
Vorhees & Williams (2006) Morris water maze: procedures for assessing spatial and related forms of learning and memory. Nat Protoc 1:848-858.
Wang et al. (2017) IL-17 induced NOTCHI activation in oligodendrocyte progenitor cells enhances proliferation and inflammatory gene expression. Nat Commun 8:15508.
Wang et al. (2019) MicroRNAs in Alzheimer's Disease. Front Genet 10:153.
Wischik et al. (1996) Selective inhibition of Alzheimer disease-like tau aggregation by phenothiazines. Proc Natl Acad Sci USA 93:11213-11218.
Witmer et al. (2004) In vivo angiogenic phenotype of endothelial cells and pericytes induced by vascular endothelial growth factor-A. J Histochemi Cytochem 52:39-52.
Wojtas et al. (2017) Loss of clusterin shifts amyloid deposition to the cerebrovasculature via disruption of perivascular drainage pathways. Proc Natl Acad Sci USA 114:E6962-E6971.
Wojtas et al. (2020) Astrocyte-derived clusterin suppresses amyloid formation in vivo. Mol Neurodegener 15:71.
Wu (2007) Self-complementary recombinant adeno-associated viral vectors: packaging capacity and the role of rep proteins in vector purity. Hum Gene Ther 18(2):171-182.
Wyatt et al. (2011) Clusterin facilitates in vivo clearance of extracellular misfolded proteins. Cell Mol Life Sci 68:3919-3931.
Xiao et al. (1998) Adeno-associated virus as a vector for liver-directed gene therapy. J Virol 72:10222-10226.
Xiao et al. (2017) Ozone oil promotes wound healing by increasing the migration of fibroblasts via PI3K/Akt/mTOR signaling pathway. Biosci Rep 37:BSR20170658.
Xin & Chan (2020) Myelin plasticity: sculpting circuits in learning and memory. Nat Rev Neurosci 21:682-694.
Yang et al. (1994) Characterization of cell lines that inducibly express the adeno-associated virus Rep proteins. J Virol 68:4847-4856.
Yang et al. (1995) Cellular and humoral immune responses to viral antigens create barriers to lung-directed gene therapy with recombinant adenoviruses. J Virol 69:2004-2015.
Yao et al. (2014) Olig2 overexpression accelerates the differentiation of mouse embryonic stem cells into oligodendrocyte progenitor cells in vitro. Dev Growth Differ 56:511-517.
Zhan et al. (1995) Distribution of beta amyloid associated proteins in plaques in Alzheimer's disease and in the non-demented elderly. Neurodegeneration 4:291-297.
Zhan et al (2014) Myelin Injury and Degraded Myelin Vesicles in Alzheimer's Disease. Curr Alzheimer Res 11:232-238.
Zhang et al. (2006) Oligodendrocyte progenitor cells derived from human embryonic stem cells express neurotrophic factors. Stem Cells Dev 15:943-952.
Zhang et al. (2019) Senolytic therapy alleviates AP-associated oligodendrocyte progenitor cell senescence and cognitive deficits in an Alzheimer's disease model. Nat Neurosci 22:719-728.
Zheng et al. (2000) Genomic integration and gene expression by a modified adenoviral vector. Nature Biotechnol 18:176-180.

It will be understood that various details of the presently disclosed subject matter can be changed without departing from the scope of the presently disclosed subject matter. Furthermore, the foregoing description is for the purpose of illustration only, and not for the purpose of limitation.

TABLE 2
Exemplary pre-miRNAs and Mature miRNAs of
the Presently Disclosed Subject Matter

| | | | SEQ | | SEQ |
|---|---|---|---|---|---|
| pre-miRNA¹ | Gene Symbol | Accession² | ID NO: | mature miRNA^{1, 3} | ID NO: |
| hsa-miR-15a | MIR15A | NR_029485.1 | 12 | hsa-miR-15a-Sp | 88 |
| mmu-miR-15a | Mir15a | NR_029733.1 | 13 | mmu-miR-15a-5p | 89 |
| hsa-miR-15b | MIR15B | NR_029663.1 | 14 | hsa-miR-15b-5p | 90 |
| hsa-miR-16 | MIR16-1 | NR_029486.1 | 15 | hsa-miR-16-5p | 91 |
| hsa-miR-17 | MIR17 | NR_029487.1 | 16 | hsa-miR-17-5p | 92 |
| hsa-miR-18a | MIR18A | NR_029488.1 | 17 | hsa-miR-18a-3p | 93 |
| hsa-miR-21 | MIR21 | NR_029493.1 | 18 | hsa-miR-21-5p | 94 |
| hsa-miR-25 | MIR25 | NR_029498.1 | 19 | hsa-miR-25-5p | 95 |
| hsa-miR-29b-1 | MIR29B1 | NR_029517.1 | 20 | hsa-miR-29b-1 | 96 |
| hsa-miR-29b-2 | MIR29B2 | NR_029518.1 | 21 | hsa-miR-29b-2a | 97 |
| | | | | hsa-miR-29b-2b | 98 |
| hsa-miR-126 | MIR126 | NR_029695.1 | 22 | hsa-miR-126a | 99 |
| | | | | hsa-miR-126b | 100 |
| hsa-miR-145 | MIR145 | NR_029686.1 | 23 | hsa-miR-145a | 101 |
| | | | | hsa-miR-145b | 102 |
| hsa-miR-195 | MIR195 | NR_029712.1 | 24 | hsa-miR-195-5p | 103 |
| mmu-miR-301b | Mir301b | NR_030415.1 | 25 | mmu-miR-301b-3p | 104 |
| hsa-miR-326 | MIR326 | NR_029891.1 | 26 | hsa-miR-326 | 105 |
| hsa-miR-330 | MIR330 | NR_029886. | 27 | hsa-miR-330-5p | 106 |
| hsa-miR-424 | MIR424 | NR_029946.1 | 28 | hsa-miR-424-5p | 107 |
| hsa-miR-425 | MIR425 | NR_029948.1 | 29 | hsa-miR-425-5p | 108 |
| hsa-miR-485 | MIR485 | NR_030160.1 | 30 | hsa-miR-485-5p | 109 |
| hsa-miR-497 | MIR497 | NR_030178.1 | 31 | hsa-miR-497-5p | 110 |
| hsa-miR-518c | MIR518C | NR_030199.1 | 32 | hsa-miR-518c-5p | 111 |
| hsa-miR-520a | MIR520A | NR_030189.1 | 33 | hsa-miR-520a-5p | 112 |
| hsa-miR-525 | MIR525 | NR_030192.1 | 34 | hsa-miR-525-5p | 113 |
| hsa-miR-571 | MIR571 | NR_030297.1 | 35 | hsa-miR-571 | 114 |
| hsa-miR-650 | MIR650 | NR_030755.1 | 36 | hsa-miR-650 | 115 |
| hsa-miR-873 | MIR873 | NR_030618.1 | 37 | hsa-miR-873-5p | 116 |
| hsa-miR-890 | MIR890 | NR_030589.1 | 38 | hsa-miR-890 | 117 |
| hsa-miR-922 | MIR922 | NR_030627.1 | 39 | hsa-miR-922 | 118 |
| hsa-miR-1228 | MIR1228 | NR_031597.1 | 40 | hsa-miR-1228-5p | 119 |
| hsa-miR-1237 | MIR1237 | NR_031602.1 | 41 | hsa-miR-1237-5p | 120 |
| hsa-miR-1254 | MIR1254-1 | NR_031655.1 | 42 | hsa-miR-1254 | 121 |
| hsa-miR-1275 | MIR1275 | NR_031681.1 | 43 | hsa-miR-1275 | 122 |
| hsa-miR-1286 | MIR1286 | NR_031618.1 | 44 | hsa-miR-1286 | 123 |
| hsa-miR-2467 | MIR2467 | NR_039948.1 | 45 | hsa-miR-2467-3p | 124 |
| hsa-miR-2861 | MIR2861 | NR_036055.1 | 46 | hsa-miR-2861 | 125 |
| hsa-miR-3116-1 | MIR3116-1 | NR_036058.1 | 47 | hsa-miR-3116 | 126 |
| hsa-miR-3116-2 | MIR3116-2 | NR_036059.1 | 48 | | |
| hsa-miR-3152 | MIR3152 | NR_036107.1 | 49 | hsa-miR-3152-5p | 127 |
| | | | | hsa-miR-3152-3p | 128 |
| hsa-miR-3175 | MIR3175 | NR_036136.1 | 50 | hsa-miR-3175 | 129 |
| hsa-miR-3191 | MIR3191 | NR_036159.1 | 51 | hsa-miR-3191-5p | 130 |
| hsa-miR-3612 | MIR3612 | NR_037406.1 | 52 | hsa-miR-3612 | 131 |
| hsa-miR-3659 | MIR3659 | NR_037432.1 | 53 | hsa-miR-3659 | 132 |
| hsa-miR-3678 | MIR3678 | NR_037449.1 | 54 | hsa-miR-3678-3p | 133 |
| hsa-miR-4254 | MIR4254 | NR_036216.1 | 55 | hsa-miR-4254 | 134 |
| hsa-miR-4259 | MIR4259 | NR_130464.1 | 56 | hsa-miR-4259 | 135 |
| hsa-miR-4443 | MIR4443 | NR_039645.1 | 57 | hsa-miR-4443 | 136 |
| hsa-miR-4486 | MIR4486 | NR_039706.1 | 58 | hsa-miR-4486 | 137 |
| hsa-miR-4488 | MIR4488 | NR_039708.1 | 59 | hsa-miR-4488 | 138 |
| hsa-miR-4505 | MIR4505 | NR_039727.1 | 60 | hsa-miR-4505 | 139 |
| hsa-miR-4661 | MIR4661 | NR_039805.1 | 61 | hsa-miR-4661-3p | 140 |
| hsa-miR-4665 | MIR4665 | NR_039811.1 | 62 | hsa-miR-4665-5p | 141 |
| hsa-miR-4697 | MIR4697 | NR_039846.1 | 63 | hsa-miR-4697-5p | 142 |
| hsa-miR-4706 | MIR4706 | NR_039855.1 | 64 | hsa-miR-4706 | 143 |
| hsa-miR-4722 | MIR4722 | NR_039873.1 | 65 | hsa-miR-4722-5p | 144 |
| hsa-miR-4731 | MIR4731 | NR_039884.1 | 66 | hsa-miR-4731-5p | 145 |
| hsa-miR-4749 | MIR4749 | NR_039904.2 | 67 | hsa-miR-4749-5p | 146 |
| hsa-miR-4781 | MIR4781 | NR_039942.1 | 68 | hsa-miR-4781-5p | 147 |
| hsa-miR-5586 | MIR5586 | NR_049851.1 | 69 | hsa-miR-5586-3p | 148 |
| hsa-miR-5787 | MIR5787 | NR_106714.1 | 70 | hsa-miR-5787 | 149 |
| hsa-miR-6731 | MIR6731 | NR_106789.1 | 71 | hsa-miR-6731-5p | 150 |
| hsa-miR-6751 | MIR6751 | NR_106809.1 | 72 | hsa-miR-6751-5p | 151 |
| hsa-miR-6764 | MIR6764 | NR_106822.1 | 73 | hsa-miR-6764-3p | 152 |
| hsa-miR-6777 | MIR6777 | NR_106835.1 | 74 | hsa-miR-6777-5p | 153 |
| hsa-miR-6803 | MIR6803 | NR_106861.1 | 75 | hsa-miR-6803-5p | 154 |
| hsa-miR-6817 | MIR6817 | NR_106875.1 | 76 | hsa-miR-6817-3p | 155 |
| hsa-miR-6824 | MIR6824 | NR_106882.1 | 77 | hsa-miR-6824-3p | 156 |
| hsa-miR-6838 | MIR6838 | NR_106897.1 | 78 | hsa-miR-6838-5p | 157 |
| hsa-miR-6846 | MIR6846 | NR_106905.1 | 79 | hsa-miR-6846-5p | 158 |
| hsa-miR-6848 | MIR6848 | NR_106907.1 | 80 | hsa-miR-6848-5p | 159 |
| hsa-miR-6873 | MIR6873 | NR_106933.1 | 81 | hsa-miR-6873-3p | 160 |
| hsa-miR-6884 | MIR6884 | NR_106944.1 | 82 | hsa-miR-6884-5p | 161 |
| hsa-miR-6889 | MIR6889 | NR_106949.1 | 83 | hsa-miR-6889-5p | 162 |
| hsa-miR-7109 | MIR7109 | NR_106959.1 | 84 | hsa-miR-7109-5p | 163 |
| hsa-miR-7110 | MIR7110 | NR_106960.1 | 85 | hsa-miR-7110-3p | 164 |
| hsa-miR-7855 | MIR7855 | NR_107009.1 | 86 | hsa-miR-7855-5p | 165 |
| hsa-miR-8085 | MIR8085 | NR_107052.1 | 87 | hsa-miR-8085 | 166 |

¹“hsa” indicates that the pre-miRNA is of human origin and “mmu” indicates that the pre-miRNA is of murine origin.
²Accession number in the GENBANK ® biosequence database.
³Exemplary mature miRNA(s) that is/are derived from the corresponding pre-miRNA.

1-3. (canceled)

4. A method for reducing and/or inhibiting myelin decay in a subject in need thereof, the method comprising, consisting essentially of, or consisting of administering to the subject at least one composition comprising, consisting essentially of, or consisting of an inhibitor of a clusterin biological activity and/or an inducer of IL-9 biological activity, wherein the at least one composition is administered via a route and in an amount sufficient to inhibit clusterin biological activity and/or induce IL-9 biological activity in the subject to thereby reduce and/or inhibit myelin decay in the subject.

5. The method of claim 4, wherein the subject has or is at risk for developing a disease, disorder, or condition, optionally a disease, disorder, or condition selected from the group consisting of multiple sclerosis, spinal cord injury, brain injury, leukodystrophies, neuromyelitis optica (NMO), and Alzheimer's Disease (AD), or a worsening of at least one symptom thereof.

6. The method of claim 4, wherein the inhibitor of a clusterin biological activity comprises an inhibitory nucleic acid that binds to and reduces translation of a clusterin gene product, optionally a human clusterin gene product.

7. The method of claim 6, wherein the inhibitory nucleic acid targets a subsequence of a human clusterin gene product as set forth in Accession No. NM_001831.4 of the GENBANK® biosequence database (SEQ ID NO: 7).

8. The method of claim 4, wherein the inhibitor of a clusterin biological activity comprises a guide RNA (gRNA) that targets a clusterin gene product for modification with CRISPR/cas9, optionally wherein the gRNA comprises a sequence that comprises, consists essentially of, or consists of a nucleotide sequence selected from the group consisting of CGTCTATGATGCTGGACGCG (SEQ ID NO: 2), GACGTACTTACTTCCCTGAT (SEQ ID NO: 3), and GCGTGCGTAGAACTTCATGC (SEQ ID NO: 6) and/or that targets a clusterin gene product nucleotide sequence that comprises, consists essentially of, or consists of a nucleotide sequence selected from the group consisting of TACGCACGCGTCTGCAGAAG (SEQ ID NO: 1), AGAAGGCGACGATGAC (SEQ ID NO: 4), and CCGCCAACAGAATTCATACG (SEQ ID NO: 5).

9. (canceled)

10. A method for inhibiting differentiation of oligodendrocyte progenitor cells (OPCs) to mature oligodendrocytes, the method comprising, consisting essentially of, or consisting of contacting the OPCs with a clusterin gene product or a functional fragment or derivative thereof.

11. The method of claim 10, wherein the clusterin gene product comprises SEQ ID NO: 8 or a post-translationally modified subsequence thereof.

12. The method of claim 4, further comprising administering at least one additional therapy, optionally selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; a nucleotide reverse transcriptase inhibitor (NRTI), optionally an NRTI abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, a non-nucleoside reverse transcriptase inhibitor (NNRTI), optionally an NNRTI selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof.

13. The method of claim 12, wherein the at least one additional therapy comprises treatment with an acetylcholinesterase (AChE) inhibitor, optionally wherein the AChE inhibitor is selected from the group consisting of donepezil, rivastigmine, and galantamine.

14. (canceled)

15. The method of claim 12, wherein the at least one additional therapy comprises treatment with an N-methyl-d-aspartate receptor (NMDAR) antagonist, optionally memantine.

16. (canceled)

17. The method of claim 12, wherein the at least one additional therapy comprises treatment with a secretase inhibitor.

18. The method of claim 12, wherein the at least one additional therapy comprises treatment with a beta-site APP-cleaving enzyme (BACE) inhibitor.

19. The method of claim 12, wherein the at least one additional therapy comprises treatment with an inhibitor of tau aggregation.

20. The method of claim 12, wherein the at least one additional therapy comprises treatment with an inhibitory nucleic acid, optionally wherein the inhibitory nucleic acid is an miRNA selected from the group consisting of miR-126, miR-145, miR-195, miR-21, and miR-29b.

21. (canceled)

22. The method of claim 12, wherein the at least one additional therapy comprises treatment with a nucleotide reverse transcriptase inhibitor (NRTI) and/or a non-nucleoside reverse transcriptase inhibitor (NNRTI), optionally wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, and pharmaceutically acceptable salts thereof, and/or the NNRTI is optionally selected from the group consisting of delavirdine (DLV), efavirenz (EFV), etravirine (ETR), nevirapine (NVP, rilpivirine (TMC278), doravirine (MK-1439), derivatives thereof, and pharmaceutically acceptable salts thereof.

23-28. (canceled)

29. The method of claim 4, wherein the additional composition comprises, consists essentially of, or consists of a biologically active IL-9 polypeptide, a biologically active fragment thereof, a vector encoding a biologically active IL-9 polypeptide and/or a biologically active fragment thereof, optionally a viral vector, further optionally an adeno-associated virus (AAV) vector, a small molecule that induces IL-9 biological activity, an IL-9 receptor agonist, and/or a genetic construct that induces IL-9 biological activity in the subject in need thereof.

30. A method for treating a disease, disorder, or condition associated with undesirable demyelination and/or ameliorating at least one symptom thereof, the method comprising, consisting essentially of, or consisting of administering to a subject with a disease, disorder, or condition associated with undesirable demyelination one or more compositions that individually or together comprise, consist essentially of, or consist of:
(a) an inhibitor of a clusterin biological activity; and/or
(b) an inducer of IL-9 biological activity;
wherein the at least one composition is administered via a route and in an amount sufficient to inhibit clusterin biological activity and/or induce IL-9 biological activity in the subject to thereby treat the subject's disease, disorder, or condition and/or to ameliorate at least one symptom thereof.

31. The method of claim 30, wherein the disease, disorder, or condition is selected from the group consisting of multiple sclerosis; spinal cord injury, brain injury, leukodystrophies, neuromyelitis optica (NMO), and Alzheimer's Disease (AD), and optionally wherein the administering reduces an amount of clusterin and/or increases an amount of IL-9 in at least one cell type of the central nervous system (CNS) of the subject, optionally in the brain of the subject.

32. (canceled)

33. The method of claim 30, wherein the inducer of IL-9 biological activity comprises, consists essentially of, or consists of a biologically active IL-9 polypeptide, a biologically active fragment thereof, a vector encoding a biologically active IL-9 polypeptide and/or a biologically active fragment thereof, optionally a viral vector, further optionally an adeno-associated virus (AAV) vector, a small molecule that induces IL-9 biological activity, an IL-9 receptor agonist, and/or a genetic construct that induces IL-9 biological activity in the subject.

34-39. (canceled)

40. The method of claim 4, wherein the inducer of IL-9 biological activity comprises, consists essentially of, or consists of a biologically active IL-9 polypeptide, a biologically active fragment thereof, a vector encoding a biologically active IL-9 polypeptide and/or a biologically active fragment thereof, optionally a viral vector, further optionally an adeno-associated virus (AAV) vector, a small molecule that induces IL-9 biological activity, an IL-9 receptor agonist, and/or a genetic construct that induces IL-9 biological activity in the subject.
